(19) United States
(12) Patent Application Publication
(10) Pub. No.: US 2023/0091256 A1
(43) Pub. Date: Mar. 23, 2023

(54) Hidden Frame Neoantigens

(71) Applicant: CureVac Netherlands B.V., Amsterdam (NL)

(72) Inventors: Wigard Pieter KLOOSTERMAN, Bunnik (NL); Ronald Hans Anton PLASTERK, Amsterdam (NL)

(21) Appl. No.: 17/796,358

(22) PCT Filed: Feb. 26, 2021
(86) PCT No.: PCT/NL2021/050128

§ 371 Date: Jul. 29, 2022

(30) Foreign Application Priority Data
Feb. 28, 2020 (EP) 20160013.7
Jul. 6, 2020 (EP) 20184218.4
Dec. 21, 2020 (EP) 20215918.2

(51) Int. Cl. A61K 39/00; C12Q 1/6886; C12N 5/0783

(52) U.S. Cl. CPC A61K 39/0011; C12Q 1/6886; C12N 5/0636; C12Q 1/6869

(57) ABSTRACT

The invention relates to the field of cancer. In particular, it relates to the field of immune system directed approaches for tumor treatment, reduction and control. Some aspects of the invention relate to the identification of tumor specific neoantigens, such as those resulting from frameshift mutations or DNA rearrangements. Such neoantigens are useful for developing tumor treatments, such as vaccines or cellular immunotherapies and other means of stimulating a neoantigen specific immune response against a tumor in individuals. A new class of neoantigens, referred to herein as ‘Hidden Frames’, as well as methods of identifying such neoantigens is provided.

### FIELD OF THE INVENTION

The invention relates to the field of cancer. In particular, it relates to the field of immune system directed approaches for tumor treatment, reduction and control. Some aspects of the invention relate to the identification of tumor specific neoantigens, such as those resulting from frameshift mutations or DNA rearrangements. Such neoantigens are useful for developing tumor treatments, such as vaccines or cellular immunotherapies and other means of stimulating a neoantigen specific immune response against a tumor in individuals. A new class of neoantigens, referred to herein as ‘Hidden Frames’, as well as methods of identifying such neoantigens is provided.

### BACKGROUND OF THE INVENTION

There are a number of different existing cancer therapies, including ablation techniques (e.g., surgical procedures and radiation) and chemical techniques (e.g., pharmaceutical agents and antibodies), and various combinations of such techniques. Despite intensive research such therapies are still frequently associated with serious risk, adverse or toxic side effects, as well as varying efficacy.

There is a growing interest in cancer therapies that aim to target cancer cells with a patient's own immune system (such as cancer vaccines or checkpoint inhibitors, or T-cell based immunotherapy). Such therapies may indeed eliminate some of the known disadvantages of existing therapies or be used in addition to the existing therapies for additional therapeutic effect. Cancer vaccines or immunogenic compositions intended to treat an existing cancer by strengthening the body's natural defenses against the cancer and based on tumor-specific neoantigens hold great promise as personalized cancer immunotherapy. Evidence shows that such neoantigen-based vaccination can elicit T-cell responses and can cause tumor regression in patients.

Typically, the immunogenic compositions/vaccines are composed of tumor antigens (antigenic peptides or nucleic acids encoding them) and may include immune stimulatory molecules like cytokines that work together to induce antigen-specific cytotoxic T-cells that target and destroy tumor cells. Many reports describe vaccination based on somatic SNVs (Single Nucleotide Variants) that lead to single amino acid changes in proteins, and hence encode new antigens (neoantigens) that are specific to the tumor. As shown in FIG. 1, on average 95% of all coding somatic mutations in the ORFeome (i.e. the entire collection of all Open Reading Frame sequences in the genome) of tumors (excluding synonymous or truncating SNVs) are missense SNVs (Single Nucleotide Variants), as based on the tumor mutation reports available for the TCGA database.

Much of the research in recent years has focused on the prediction (either in silico or by experimental analysis) of which of these many mutations would make for the best neoantigen to use as a vaccine (Schumacher, T. N., Scheper, W. & Kvistborg, P. Cancer Neoantigens. Annu. Rev. Immunol. 37, 173-200 (2019)). Recent experimental estimates suggest that about 1.6% of gene products encoded by somatic nonsynonymous single nucleotide variations form an immunogenic neoantigen (Parkhurst et al. Cancer Discov Aug. 1, 2019 9 (8) 1022-1035). On average (but widely differing per tumor type, see e.g. Priestley, P. et al. Pan-cancer whole genome analyses of metastatic solid tumors. Nature volume 575, 210-216, 2019) a tumor ORFeome contains 200 missense mutations, and the practical limit of the number of peptide vaccines that can be applied to any patient has been set anywhere between 5 and 20, so that at max only a few percent of the neoantigens caused by missense mutations can be used for vaccination (see, e.g., Keskin, D. B. et al. Neoantigen vaccine generates intratumoral T cell responses in phase Ib glioblastoma trial. Nature 565, 234-239 (2019) and Ott, P. A. et al. An immunogenic personal neoantigen vaccine for patients with melanoma. Nature 547, 217-221 (2017). Therefore, the choice of the “best” SNVs is indeed crucial. In this choice it is usually considered that the peptide containing the SNV-neoantigen needs to be presented by the MHC, so that prediction of the presentation by the MHC-type of the patient is essential. For vaccine technologies other than peptides, such as DNA or RNA encoded vaccines, the number of SNVs to be included in a vaccine may be higher than 5-20, but in none of current approaches is the complete set or even the majority of all neoantigenic amino acid sequences included (Hilf, N. et al. Actively personalized vaccination trial for newly diagnosed glioblastoma. Nature 565, 240-245 (2019).

Accordingly, there is a need for improved methods and compositions for providing subject-specific immunogenic compositions/cancer vaccines. In particular, there is a need for cancer immunogenic compositions that do not rely on predicting which individual neoantigens will be most effective in vivo. One object of the present disclosure is to take the guesswork out of neoantigen selection by identifying a large part of the tumor antigenicity. A further object of the present disclosure is to provide methods for uncovering ‘hidden’ frame neoantigens and the use of said ‘hidden’ frame neoantigens as immunogenic compositions/cancer vaccines.

### SUMMARY OF THE INVENTION

In preferred embodiments, the invention provides a method for identifying candidate neoantigen sequences, said method comprising:

a) identifying somatic genomic changes in nucleic acid sequences from at least one tumor sample from an individual afflicted with cancer, wherein the somatic genomic changes result in new open reading frames, wherein the method identifies,
i) intragenic frameshift mutations in polypeptide encoding sequences, wherein the mutation results in a change of the reading frame of said polypeptide encoding sequence,
ii) DNA rearrangements resulting in new junctions of DNA sequences, wherein the DNA rearrangement results in the fusion of at least part of the coding strand of a first gene to at least part of the coding strand of a second gene or the DNA rearrangement is an intragenic DNA rearrangement (preferably selected from but not limited to intragenic deletions, intragenic tandem duplications, intragenic dispersed duplications, intragenic inverted duplications, intragenic insertions, and intragenic inversions), and
iii) DNA rearrangements resulting in new junctions of DNA sequences, wherein the rearrangement results in the fusion at least part of the coding strand of a first gene to intergenic non-coding DNA or to the non-coding strand of a second gene, preferably wherein said intergenic non-coding DNA or non-coding strand comprises a (cryptic) poly-(A) signal,

b) determining the predicted amino acid sequences encoded by the new open reading frames,

c) selecting, as candidate neoantigen peptide sequences, amino acid sequences comprising at least 9 amino acids, wherein the neoantigen peptide sequences comprise at least four contiguous amino acids encoded by the new open reading frames.

Preferably, step a) comprises identifying essentially all somatic genomic changes detectable from nucleic acid sequences obtained by whole genome sequencing.

Preferably, the method comprises performing whole genome sequencing of a tumor sample from the individual, preferably further comprising performing whole genome sequencing of a healthy (non-tumor) sample from the individual.

Preferably, the method comprises sequencing RNA from at least one tumor sample from the individual to determine the presence of RNA encoding the candidate neoantigen peptide sequences of any of the preceding claims, preferably selecting as candidate neoantigen peptide sequences, those peptide sequences whose corresponding RNA sequence is present in the tumor sample.

Preferably, the method comprises determining the sequence of the RNA overlapping the new junctions of DNA sequences resulting from said DNA rearrangements and/or the sequence of the RNA overlapping the frameshift mutation.

Preferably, determining the predicted amino acid sequences encoded by the new open reading frames comprises:

d) performing RNA sequencing on RNA from at least one tumor sample from the individual to determine the structure of RNA transcripts corresponding to the somatic genomic changes in the genomic sequences of a),

e) determining the predicted amino acid sequence encoded by the RNA transcript structure of d), and

f) selecting, as candidate neoantigen sequences, sequences comprising at least 9 contiguous amino acids of the predicted amino acid sequence of e), wherein the neoantigen peptide sequences comprise at least four contiguous amino acids encoded by the new open reading frames.

Preferably, RNA sequencing is performed using long-read direct RNA sequencing, or long-read cDNA sequencing, preferably the sequencing is performed on a Nanopore sequencing instrument. Preferably, the method further comprises selecting poly-(A) and/or Capped mRNA from said tumor sample and performing direct long-read RNA sequencing or first preparing cDNA and performing long-read sequencing on said cDNA based on the poly-(A)- and/or Cap-selected mRNA.

The methods for identifying candidate neoantigen sequences may also be performed as follows.

In some embodiments a method is provided for identifying candidate neoantigen sequences, said method comprising:

a) performing whole genome sequencing of a tumor sample and a healthy tissue sample from the individual,
optionally performing long-read whole genome sequencing of a tumor sample and a healthy tissue sample from the individual,

b) performing long-read RNA sequencing on RNA or long-read sequencing on the corresponding cDNA from at least one tumor sample from the individual (e.g., to obtain RNA sequencing reads), preferably wherein RNA is poly-(A) selected mRNA and/or 5′-CAP containing mRNA;

c) optionally performing short-read RNA sequencing on RNA or short-read sequencing on the corresponding cDNA from at least one tumor sample, preferably wherein RNA is poly-(A) selected mRNA and/or 5′-CAP containing mRNA;

d) mapping the genomic sequences obtained from the tumor tissue and corresponding healthy tissue to a human reference sequence to identify structural genomic variations in the tumor sample,

e) generating in silico a reconstructed tumor-specific reference genome comprising the identified somatic structural genomic variations;

f) aligning the RNA sequences (e.g., aligning the RNA sequencing reads) to the reconstructed tumor genome;

g) determining the sequences of the full-length RNA transcripts encoded by nucleic acid sequences comprising the somatic structural genomic variations; and

h) determining the predicted amino acid sequences encoded by the full-length transcripts of g).

In preferred embodiments the method further comprises:

i) selecting, as candidate neoantigen sequences, sequences comprising at least 9 contiguous amino acids of the predicted amino acid sequence of h), wherein at least four of the contiguous amino acids are not encoded in the germline genome of the individual.

In some embodiments a method is provided for identifying candidate neoantigen sequences, said method comprising:
performing whole genome sequencing of a tumor sample and a healthy sample from the individual,
optionally performing long-read whole genome sequencing of a tumor sample and a healthy sample from the individual,
performing long-read RNA sequencing on RNA or long-read sequencing on the corresponding cDNA from at least one tumor sample, preferably wherein RNA is poly-(A) selected mRNA and/or 5′ cap containing mRNA;
optionally performing short-read RNA sequencing on RNA or short-read sequencing on the corresponding cDNA from at least one tumor sample, preferably wherein RNA is poly-(A) selected mRNA and/or 5′ cap containing mRNA;
identifying somatic DNA rearrangements (i.e., structural genomic variations) in the tumor sample;
determining the sequence of the full-length RNA transcripts encoded by nucleic acid sequences comprising the somatic DNA rearrangements (i.e., structural genomic variations);
determining the (predicted amino acid) sequences encoded by the full-length transcripts,
selecting, as candidate neoantigen sequences, sequences comprising at least 9 contiguous amino acids of the predicted amino acid sequence encoded by the full-length transcripts, wherein at least four of the contiguous amino acids are not encoded in the germline genome of the individual.

The methods disclosed herein identify mRNA transcripts resulting from DNA rearrangements that form new junctions of DNA sequences, wherein the DNA rearrangement results in the fusion at least part of the coding strand of a first gene to intergenic non-coding DNA or to the noncoding strand of a second gene. The methods disclosed herein may also be used to identify
mRNA transcripts derived from intragenic frameshift mutations in polypeptide encoding sequences, wherein the frameshift mutation results in a change of the reading frame of said polypeptide encoding sequence, and/or
mRNA transcripts resulting from DNA rearrangements that form new junctions of DNA sequences, wherein the DNA rearrangement results in the fusion of at least part of the coding strand of a first gene to at least part of the coding strand of a second gene or the rearrangement is an intragenic genomic rearrangement, wherein said DNA rearrangement results in a change of the reading frame of a polypeptide encoding sequence.

In some embodiments of the methods an in silico a reconstructed tumor-specific reference genome is generated, said method comprising:

a) performing whole genome sequencing of a tumor sample and a healthy sample from the individual,
optionally performing long-read whole genome sequencing of a tumor sample and a healthy sample from the individual,

b) performing long-read RNA sequencing on RNA or long-read sequencing on the corresponding cDNA from at least one tumor sample to obtain RNA sequencing reads, preferably wherein RNA is poly-(A) selected mRNA and/or 5′ cap containing mRNA;

c) optionally performing short-read RNA sequencing on RNA or short-read sequencing on the corresponding cDNA from at least one tumor sample, preferably wherein RNA is poly-(A) selected mRNA and/or 5′ cap containing mRNA;

d) mapping the genomic sequences obtained from the tumor tissue and corresponding healthy tissue to a human reference sequence to identify structural genomic variations in the tumor sample,

e) generating in silico a reconstructed tumor-specific reference genome comprising the identified DNA rearrangements (i.e., somatic structural genomic variations);

f) aligning the RNA sequencing reads to the reconstructed tumor specific reference genome;

g) determining the sequences of the full-length RNA transcripts encoded by nucleic acid sequences comprising the DNA rearrangements (i.e., somatic structural genomic variations);

h) determining the predicted amino acid sequences encoded by the full-length transcripts of g),

i) selecting, as candidate neoantigen sequences, sequences comprising at least 9 contiguous amino acids of the predicted amino acid sequence of h), wherein at least four of the contiguous amino acids are not encoded in the germline genome of the individual.

In some embodiments of the methods direct RNA frame detection is used, said method comprising:

a) performing whole genome sequencing of a tumor sample and a healthy sample from the individual,
optionally performing long-read whole genome sequencing of a tumor sample and a healthy sample from the individual,

b) performing long-read RNA sequencing on RNA or long-read sequencing on the corresponding cDNA from at least one tumor sample to obtain RNA sequencing reads, preferably wherein RNA is poly-(A) selected mRNA and/or 5′ cap containing mRNA;

c) optionally performing short-read RNA sequencing on RNA or short-read sequencing on the corresponding cDNA from at least one tumor sample, preferably wherein RNA is poly-(A) selected mRNA and/or 5′ cap containing mRNA;

d) aligning the RNA sequencing reads to a human reference sequence;

e) mapping the genomic sequences obtained from the tumor tissue and corresponding healthy tissue to a human reference sequence to identify DNA rearrangements (i.e., structural genomic variations) in the tumor sample,

f) identification of a linear contig of DNA sequence from the tumor genomic sequences that comprises a DNA rearrangement (i.e., structural genomic variation) and comprises genomic segments that align to RNA sequencing reads;

g) generating in silico a reconstructed tumor-specific reference genome comprising the identified somatic structural genomic variations to which the RNA sequencing reads align;

h) aligning the RNA sequencing reads to the reconstructed tumor-specific reference genome;

i) determining the sequences of the full-length RNA transcripts encoded by nucleic acid sequences comprising the DNA rearrangement (i.e., structural genomic variation);

j) determining the amino acid sequences encoded by the full-length transcripts of i), and

k) selecting, as candidate neoantigen sequences, sequences comprising at least 9 contiguous amino acids of the amino acid sequence of j), wherein at least four of the contiguous amino acids are not encoded in the germline genome of the individual.

In preferred embodiments, the methods disclosed herein identify:
DNA rearrangements resulting in new junctions of DNA sequences, wherein the rearrangement results in the fusion of at least part of the coding strand of a first gene to at least part of the coding strand of a second gene or the rearrangement results in an intragenic genomic rearrangement (in particular wherein said DNA rearrangement results in a change of the reading frame of a polypeptide encoding sequence), and/or
DNA rearrangements resulting in new junctions of DNA sequences, wherein the rearrangement results in the fusion at least part of the coding strand of a first gene to intergenic non-coding DNA or to the noncoding strand of a second gene, preferably wherein said intergenic non-coding DNA or noncoding strand comprises a poly-(A) signal.

In preferred embodiments, the disclosure provides a method for preparing a vaccine or collection of vaccines for the treatment of cancer in an individual, comprising identifying candidate neoantigen peptide sequences according to any of the preceding embodiments and preparing a vaccine or collection of vaccines comprising peptides having said amino acid sequences or comprising nucleic acids encoding said amino acid sequences.

Preferably, the candidate neoantigen peptide sequences comprise amino acid sequences encoded by:
nucleic acid sequences comprising intragenic frameshift mutations as defined above,
nucleic acid sequences comprising DNA rearrangements that form new junctions of DNA sequences, wherein the DNA rearrangement results in the fusion of at least part of the coding strand of a first gene to at least part of the coding strand of a second gene or the rearrangement is an intragenic genomic rearrangement, wherein said DNA rearrangement results in a change of the reading frame of a polypeptide encoding sequence, and/or
nucleic acid sequences comprising DNA rearrangements that form new junctions of DNA sequences, wherein the DNA rearrangement results in the fusion at least part of the coding strand of a first gene to intergenic non-coding DNA or to the noncoding strand of a second gene (i.e., Hidden Frames). In preferred embodiments, the vaccine comprises Hidden Frame neoantigens.

Preferably, said method comprises

i) selecting from the candidate neoantigen peptide sequences identified, neoantigen peptide sequences having one or more of the following characteristics:
neoantigen peptide sequences which do not share a contiguous stretch of at least 6 amino acids with human protein reference sequences;
neoantigen peptide sequences wherein the genomic variant allele frequency of the respective somatic mutation in the tumor cells of a tumor sample is at least 0.1;
neoantigen peptide sequences wherein the cysteine content for each peptide is 30% or less, where cysteine content (Qcys) is defined as the number of cysteines in said sequence divided by the total number of amino acids in said sequence;
neoantigen peptide sequences for which the underlying somatic mutations have a maximum distance with regard to chromosomal location, preferably wherein each mutation is located on a different chromosomal arm; and
neoantigen peptide sequences wherein the peptides are predicted to comprise one or more MHC I and/or MHC II binding epitope; and

ii) preparing a vaccine or collection of vaccines comprising peptides having the selected neoantigen amino acid sequences or nucleic acids encoding the selected amino acid sequences.

Preferably, said vaccine or collection of vaccines comprises essentially all candidate neoantigen peptides identified, or nucleic acids encoding said peptides.

Preferably, the vaccine or collection of vaccines comprises at least 100 amino acids corresponding to the candidate neoantigen peptide sequences encoded by the new open reading frames.

Preferably, the vaccine or collection of vaccines comprises at least 300 or 400, preferably at least 1000, amino acids corresponding to the candidate neoantigen peptide sequences encoded by the new open reading frames.

Preferably, the cancer is not micro-satellite instable (MSI).

In a preferred embodiment, the invention provides a vaccine or collection of vaccines for the treatment of cancer, obtainable by a method as disclosed herein.

In a preferred embodiment, the invention provides a vaccine or collection of vaccines for use in the treatment of cancer in an individual. Methods are also described for treating cancer comprising administering to an individual in need thereof a vaccine or collection of vaccines as disclosed herein and/or as obtainable by a method as disclosed herein.

The invention further provides a vaccine or collection of vaccines for the treatment of cancer wherein the vaccine comprises at least two different neoantigen peptides, or nucleic acid encoding said neoantigen peptides, wherein each neoantigen is encoded by at least part of the coding strand of a first gene fused to intergenic non-coding DNA or to the noncoding strand of a second gene (herein referred to as class III frames or hidden Frames). In some embodiments, the at least two different neoantigen peptides are linked, preferably wherein said peptides are comprised within the same polypeptide. The invention further provides methods of treating an individual in need thereof with said vaccines.

In a preferred embodiment, the invention provides a method for preparing a collection of neoantigens, comprising identifying candidate neoantigen peptide sequences according to the embodiments disclosed herein and preparing peptides having said amino acid sequences or nucleic acids encoding said amino acid sequences. Preferably, said collection comprises essentially all candidate neoantigen peptide sequences identified, or nucleic acids encoding said peptides.

In a preferred embodiment, the invention provides a method for identifying candidate neoantigen sequences for an individual afflicted with cancer, said method comprising:

a) performing whole genome sequencing of a tumor sample and a healthy sample from the individual,

b) mapping the genomic sequences obtained to a human reference sequence to identify somatic genomic changes in the tumor sample, wherein the somatic genomic changes result in new open reading frames,

c) annotating the somatic genomic changes identified in the genomic sequences from the tumor sample in order to identify:
i) intragenic frameshift mutations in polypeptide encoding sequences, wherein the mutation results in a change of the reading frame of said polypeptide encoding sequence,
ii) DNA rearrangements resulting in new junctions of DNA sequences, wherein the rearrangement results in the fusion of at least part of the coding strand of a first gene to at least part of the coding strand of a second gene or the rearrangement is an intragenic genomic rearrangement (preferably selected from but not limited to intragenic deletions, intragenic tandem duplications, intragenic dispersed duplications, intragenic inverted duplications, intragenic insertions, and intragenic inversions), and
iii) DNA rearrangements resulting in new junctions of DNA sequences, wherein the rearrangement results in the fusion at least part of the coding strand of a first gene to intergenic non-coding DNA or to the noncoding strand of a second gene, preferably wherein said intergenic non-coding DNA or noncoding strand comprises a (cryptic) poly-(A) signal,

d) performing long-read RNA sequencing on poly-(A)- and/or 5′-CAP-selected RNA (or cDNA derived thereof) from at least one tumor sample from the individual to determine the structure of 5′-capped and/or polyadenylated RNA transcripts corresponding to the somatic genomic changes in the genomic sequences identified above,

optionally, performing short-read RNA sequencing on RNA (or cDNA derived thereof) from at least one tumor sample from the individual to determine the sequence of 5′-capped and/or polyadenylated RNA transcripts corresponding to the somatic genomic changes in the genomic sequences of c);

e) determining the (predicted) amino acid sequence encoded by the 5′-capped and/or polyadenylated RNA transcript structure of d),

f) selecting, as candidate neoantigen sequences, sequences comprising at least 9 contiguous amino acids of the (predicted) amino acid sequence of e), wherein at least four of the contiguous amino acids are not encoded in the germline genome of the individual.

In a preferred embodiment the neoantigen peptide or collection of neoantigen peptides can serve as a bait to select or to identify T-cells isolated from a cancer patient, or to stimulate said T-cells. In one aspect the disclosure provides a method for preparing a cellular immunotherapy for the treatment of cancer in an individual, said method comprising contacting T-cells with the candidate neoantigen peptide sequences identified from the individual according to any one of the methods described herein. Preferably, the neoantigen peptide is bound to an MHC-I molecule. In some embodiments, the T-cells are obtained from said individual. In some embodiments, contacting T-cells with the candidate neoantigen peptide sequences results in the stimulation of the T-cells. In some embodiments, the method comprises selecting T-cells having specificity for one or more of said neoantigen peptide sequences. In some embodiments, the method further comprises the in vitro expansion of the stimulated and/or selected T-cells. In some embodiments, the methods may further comprise the isolation of a T-cell receptor or a collection of T-cell receptors with specificity for one or more of said neoantigen peptide sequences.

### BRIEF DESCRIPTION OF THE DRAWINGS

FIG. 1. Average numbers of missense mutations and indel frame-shift mutations in tumor genomes. Missense mutations form the majority of neoantigenic coding mutations in cancer genomes. On average (across >10,000 tumor genomes) missense mutations occur 20 times more frequently than indel frame-shift mutations. The data are based on mutation and indel variant calls from >10,000 tumors of all major cancer types, derived from the TCGA database.

FIG. 2. The average numbers of novel amino acids encoded by missense mutations versus indel frameshift mutations in tumor genomes. On average (across >10,000 tumor genomes) frame-shift indel mutations lead to two times more novel amino acids compared to missense mutations. The data are based on mutation and indel variant calls from >10,000 tumors of all major cancer types, derived from the TCGA database. Frame predictions were performed as described (Koster, J. & Plasterk, R. H. A. A library of Neo Open Reading Frame peptides (NOPs) as a sustainable resource of common neoantigens in up to 50% of cancer patients. Sci. Rep. 9, 6577 (2019).).

FIG. 3. Schematic example of class I Frame neoantigens, caused by intra-exonic small insertions and deletions (indels).

FIG. 4. The number of novel amino acids comprised by (peptide) vaccines based on missense mutations and frameshift mutations, assuming that each vaccine may cover maximally 5-20 neoantigens derived from 5-20 mutations. The data are based on mutation and indel variant calls from >10,000 tumors of all major cancer types, derived from the TCGA database. Frameshift neoantigen predictions were performed as described (Koster, J. & Plasterk, supra).

FIG. 5. Schematic example of class II Frames, derived from out-of-frame intergenic gene fusions FIG. 6. Schematic example of class II Frames, resulting from intra-genic deletion. Similar intragenic class II Frames may result from intra-genic (tandem) duplications.

FIG. 7. Possible configurations for class II Frames.
7(A) The table depicts possible configurations of intergenic fusions between two genes, gene A (5′ fusion partner) and gene B (3′ fusion partner). The dots represent fusion configurations that may lead to a Frame neopeptide. The crosses indicate fusion configurations that are unlikely to lead to a Frame neopeptide.
7(B) Possible configurations of class II Frames where the breakpoint in the 5′ partner gene is exonic.
7(C) Possible configurations of class II Frames where the breakpoint in the 5′ partner genes is intronic.

FIG. 8. Schematic examples of Frame class III.
8(A) Class III variant 1—no splicing of read through transcript.
Class III Frames may result from genomic rearrangements where a 5′ part of a protein coding gene is fused to a segment of genomic DNA which is not known to contain a gene. Transcription of the 5′ part of the protein coding gene may cross the rearrangement breakpoint junction and lead to a new transcript that may encode a Frame neopeptide.
8(B) Class III variant 2—cryptic splicing.
Alternatively, the read-through transcription into the fused (non-coding) genomic segment may lead to novel cryptic splicing events that result in a novel mRNA encoding a Frame neopeptide.

FIG. 9. Total numbers of Frames and amino acids for Frames class I, II and III as present in 328 tumor cell lines from the CCLE collection.
9(A). Bardiagram indicating the number of Frames for each class, for both MSI-H (high level of microsatellite instability) and MSI-L (low level or no microsatellite instability) cell lines from the CCLE collection.
9(B). Bardiagram indicating the number of novel amino acids comprised by Frames for each class, for both MSI-H and MSI-L cell lines from the CCLE collection.

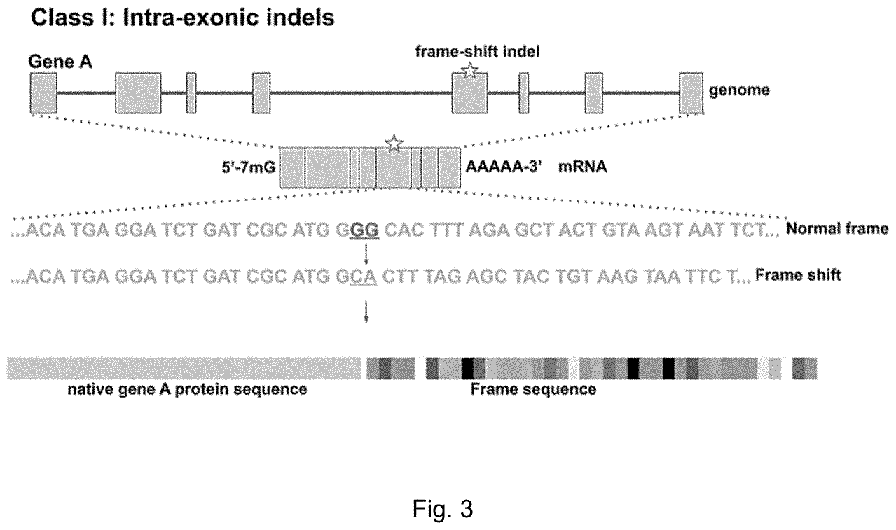

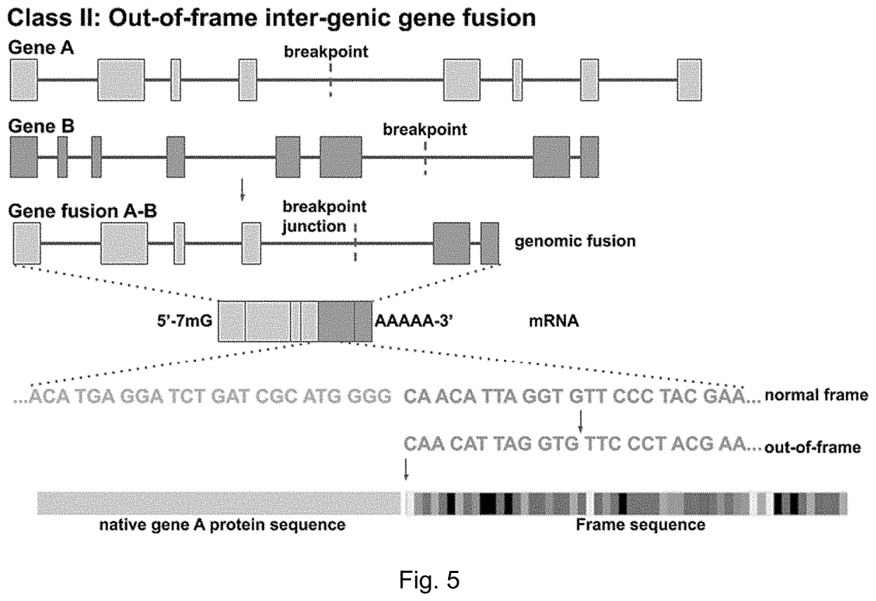

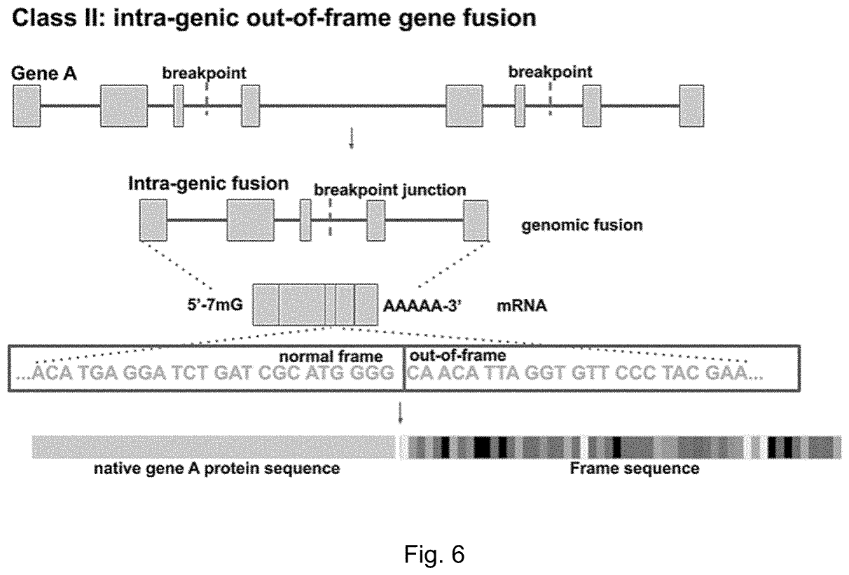

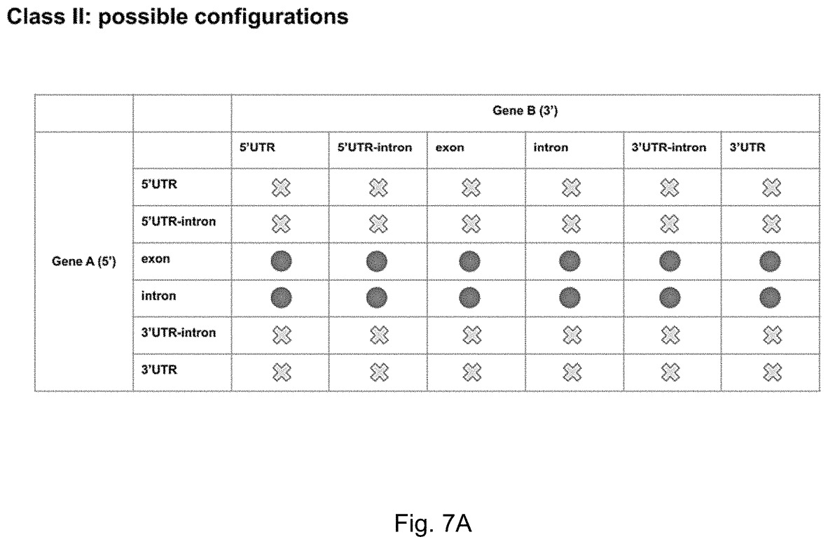

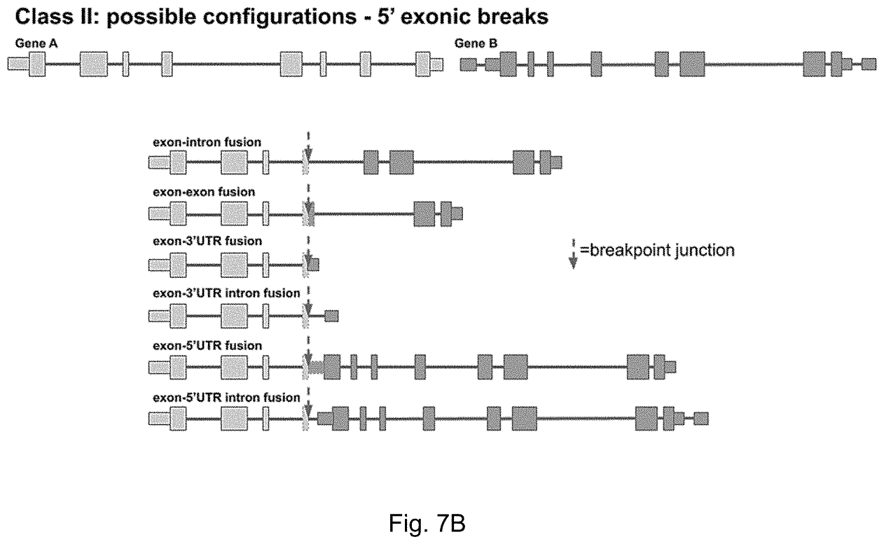

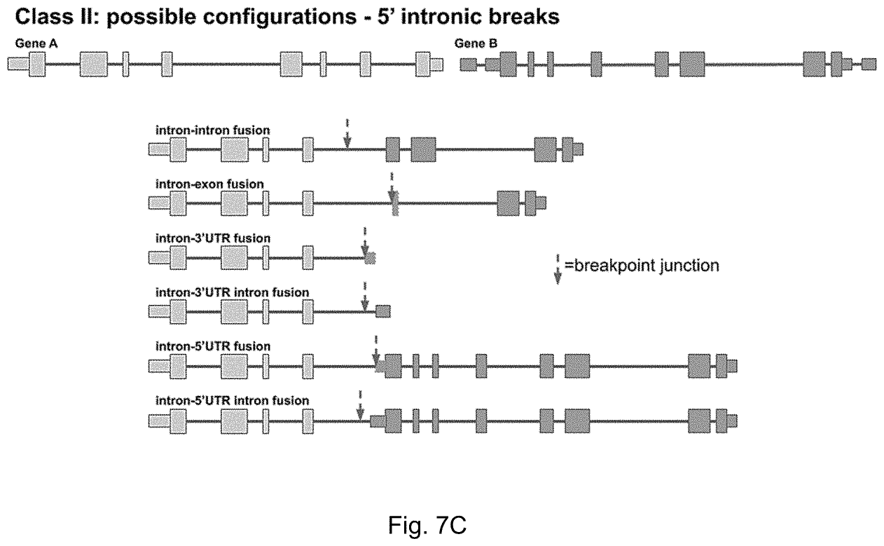

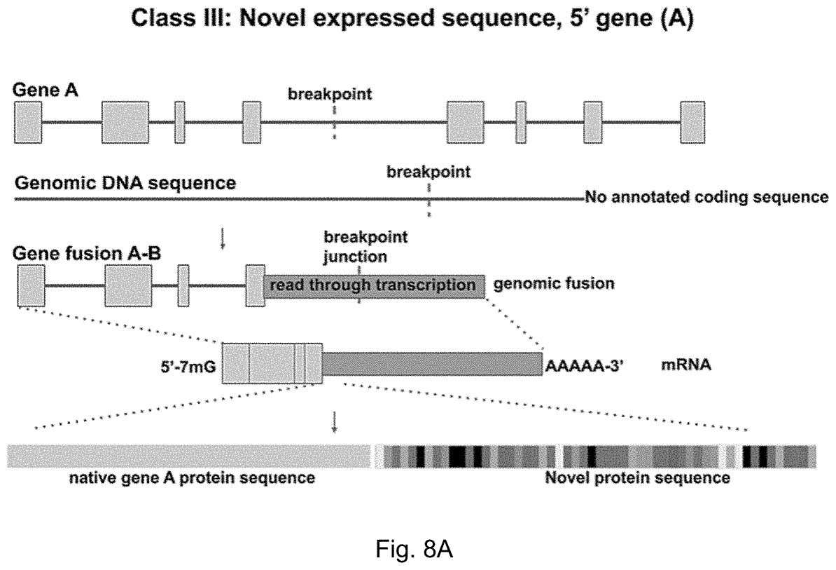

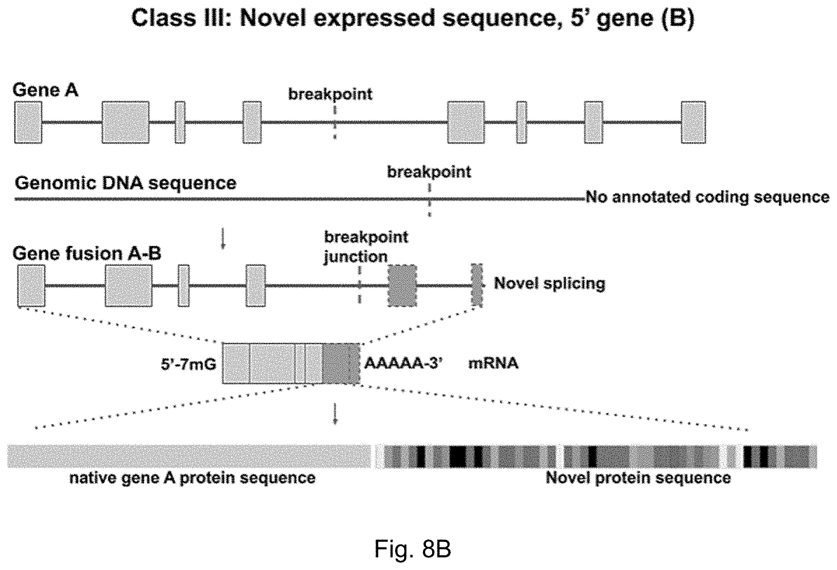

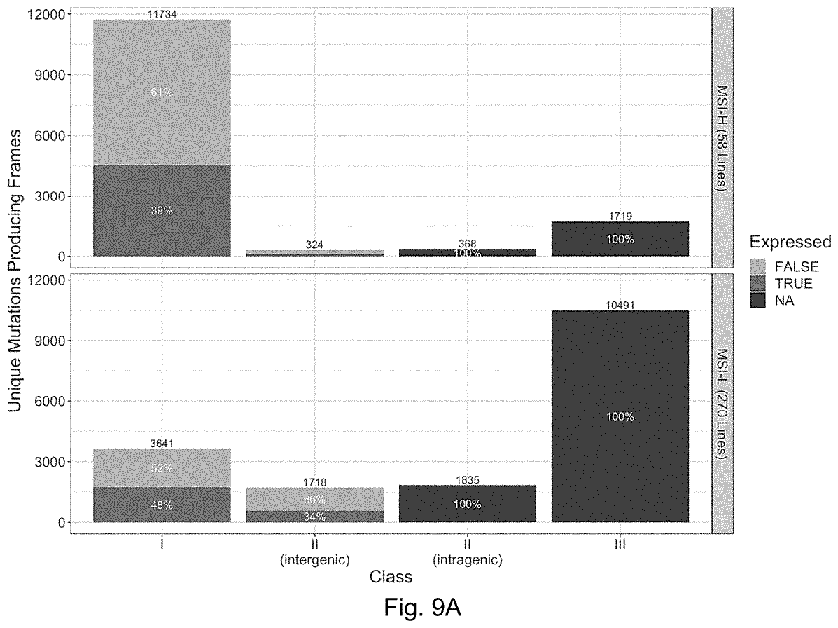

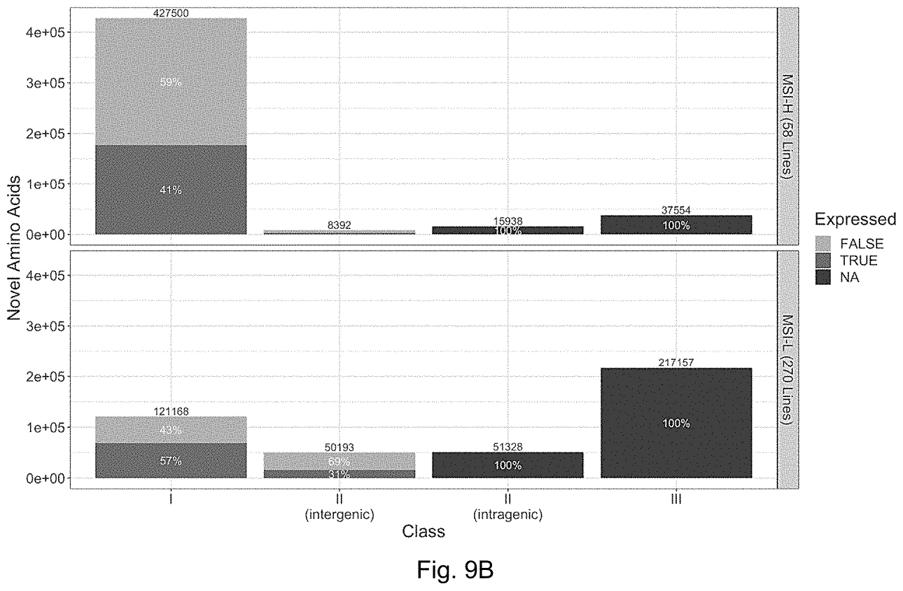

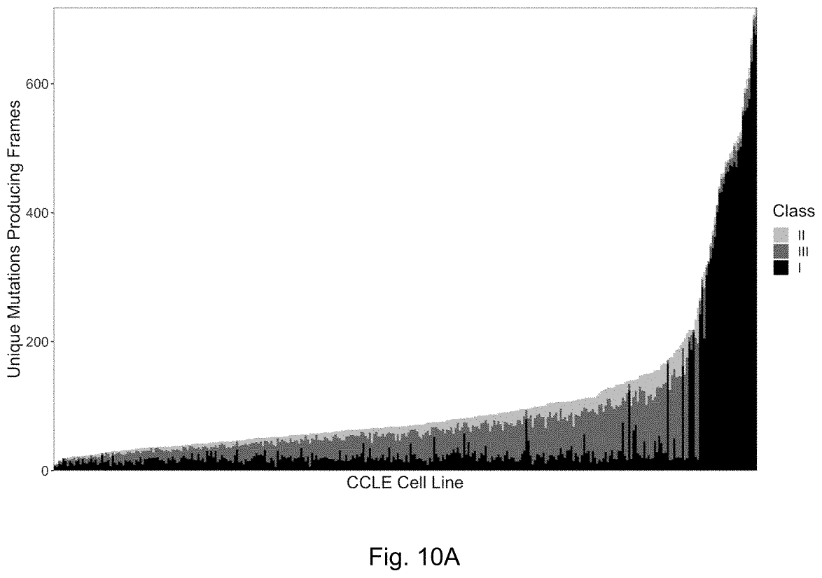

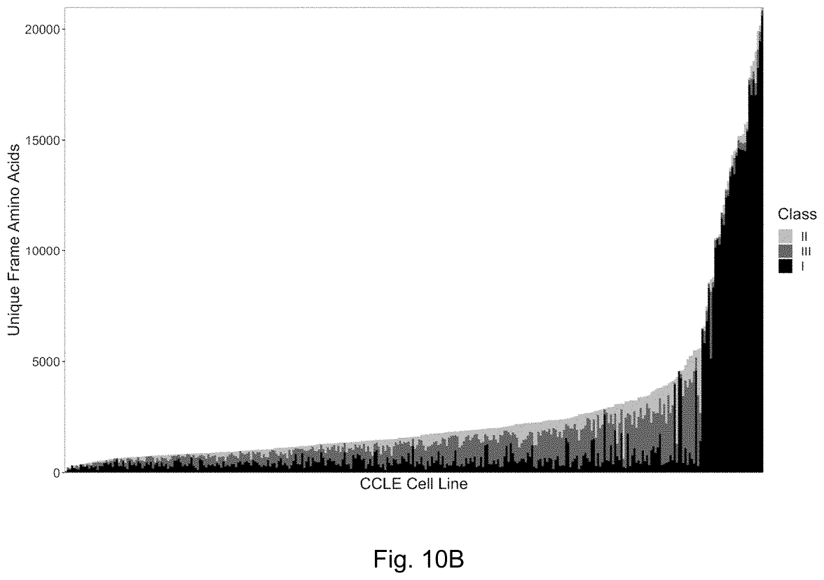

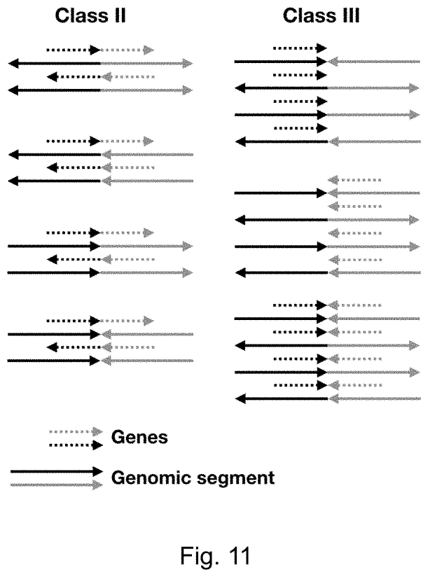

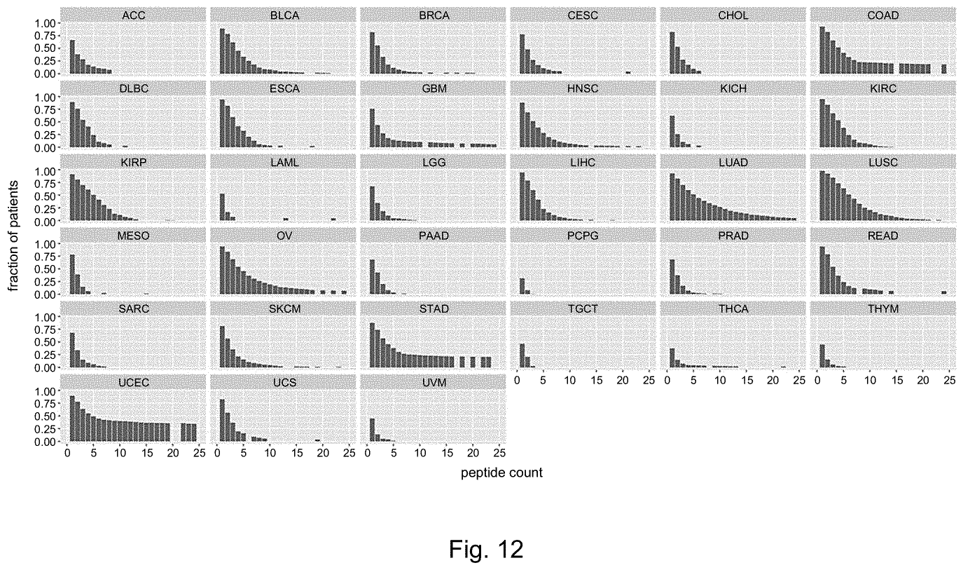

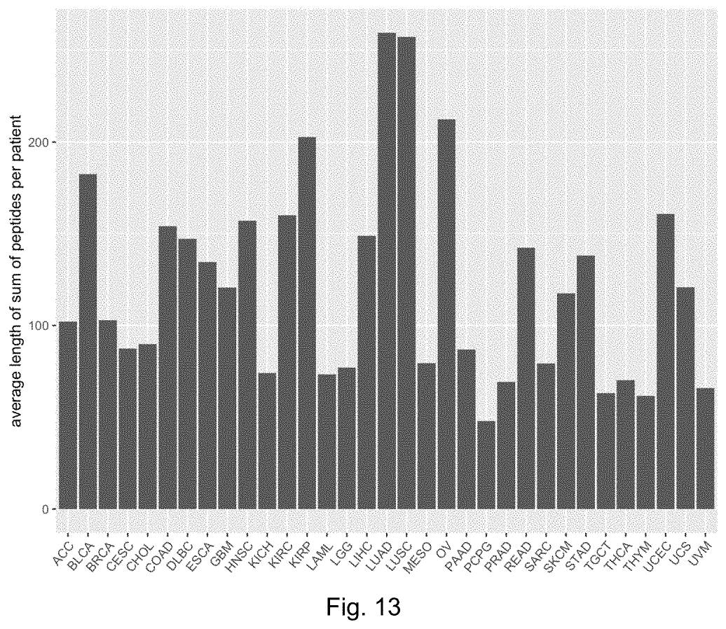

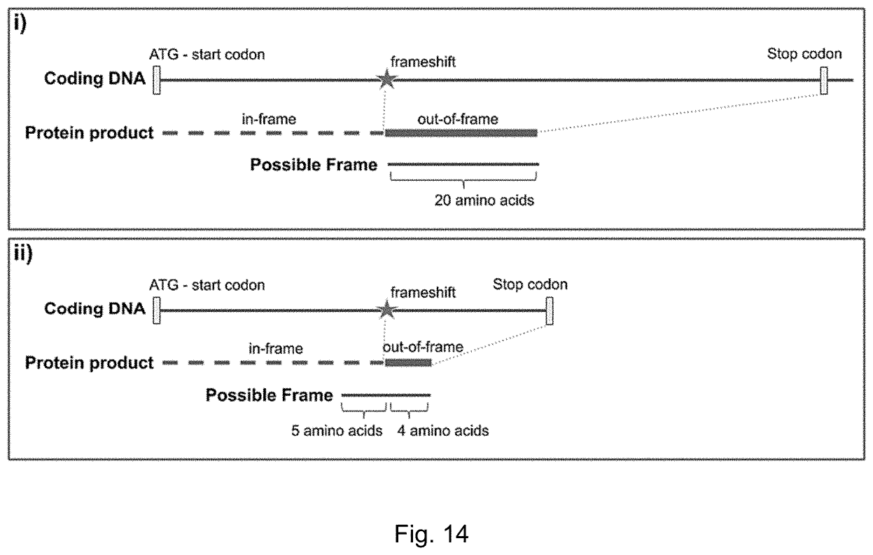

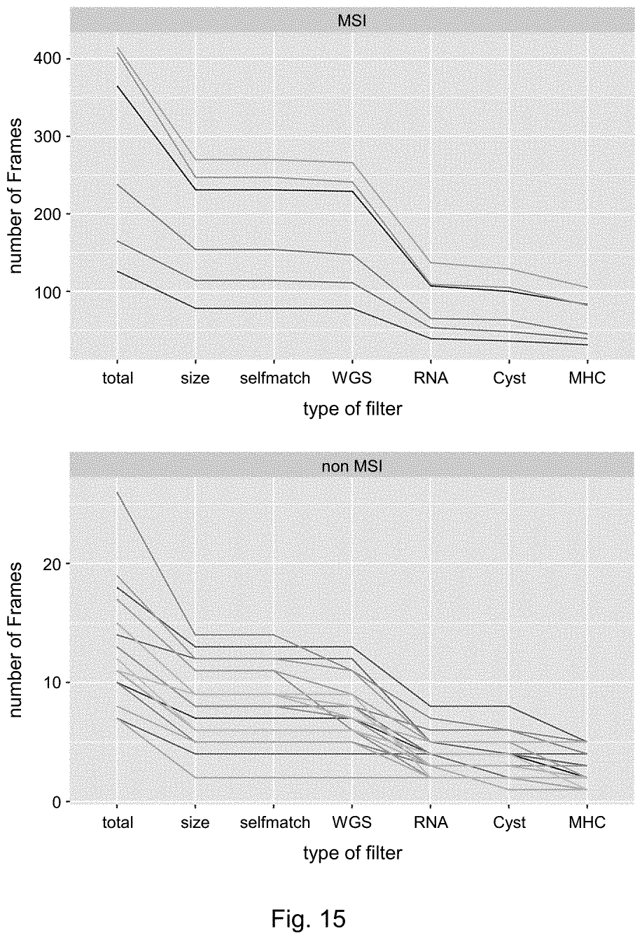

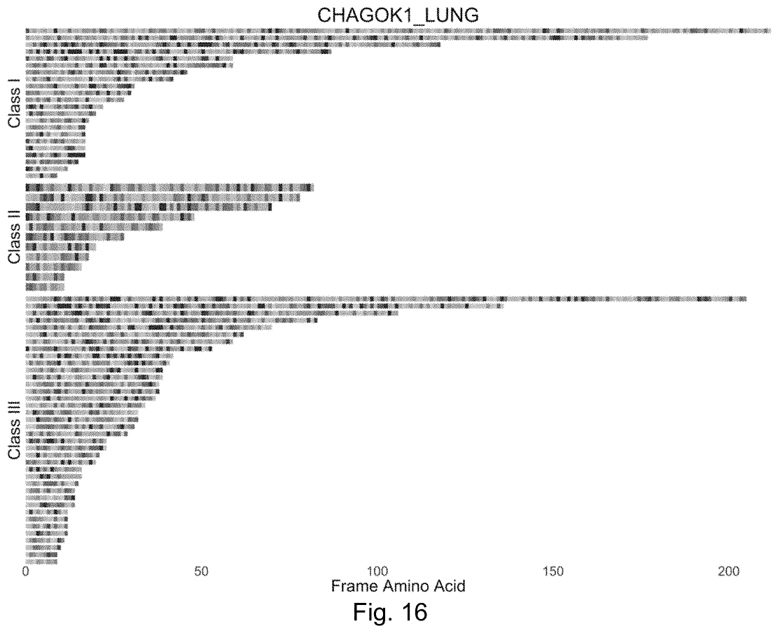

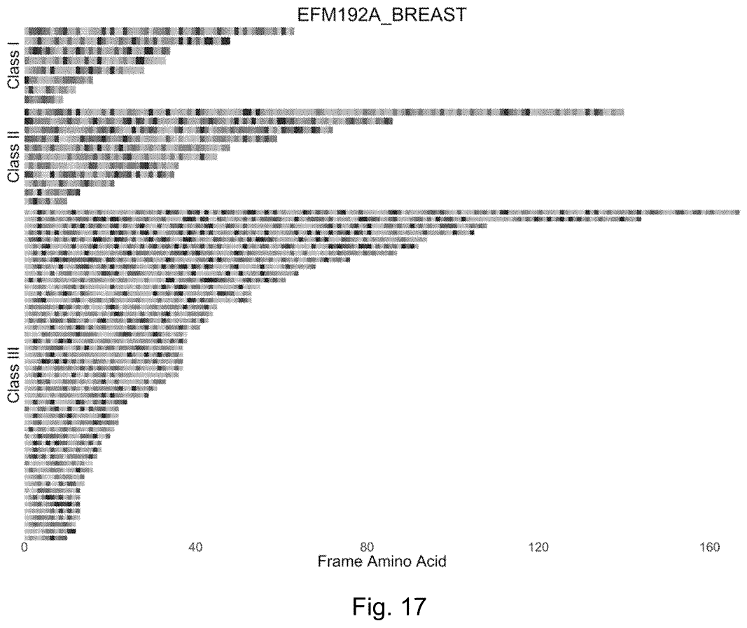

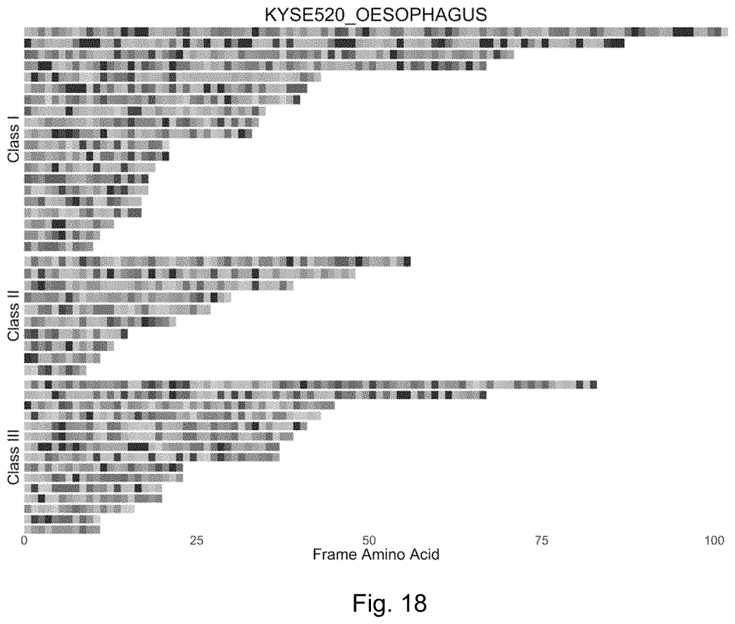

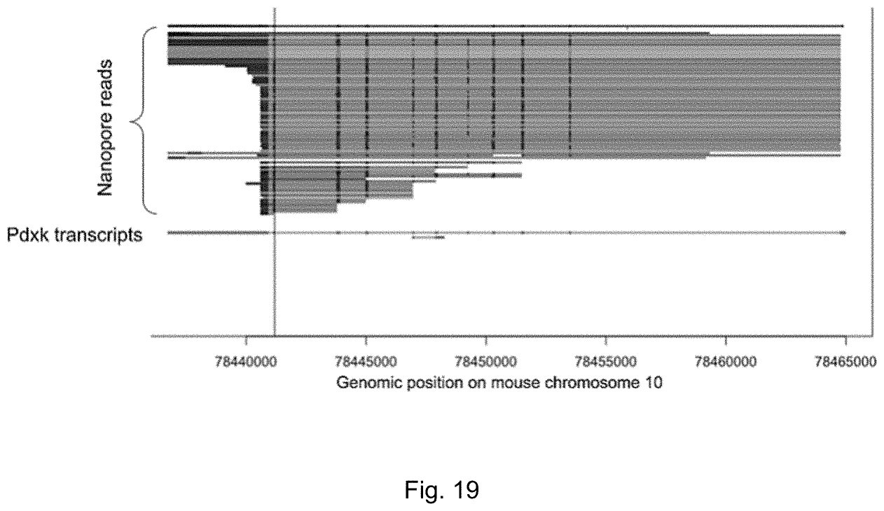

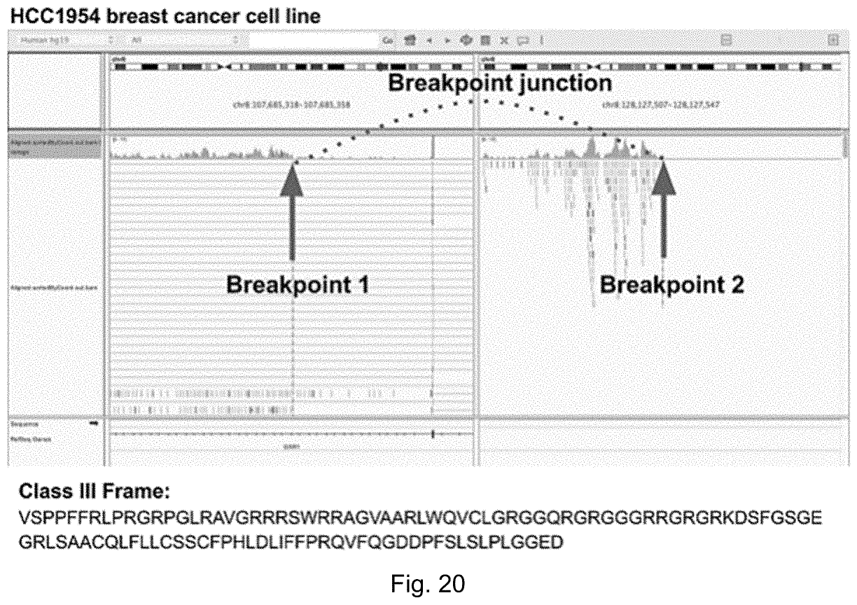

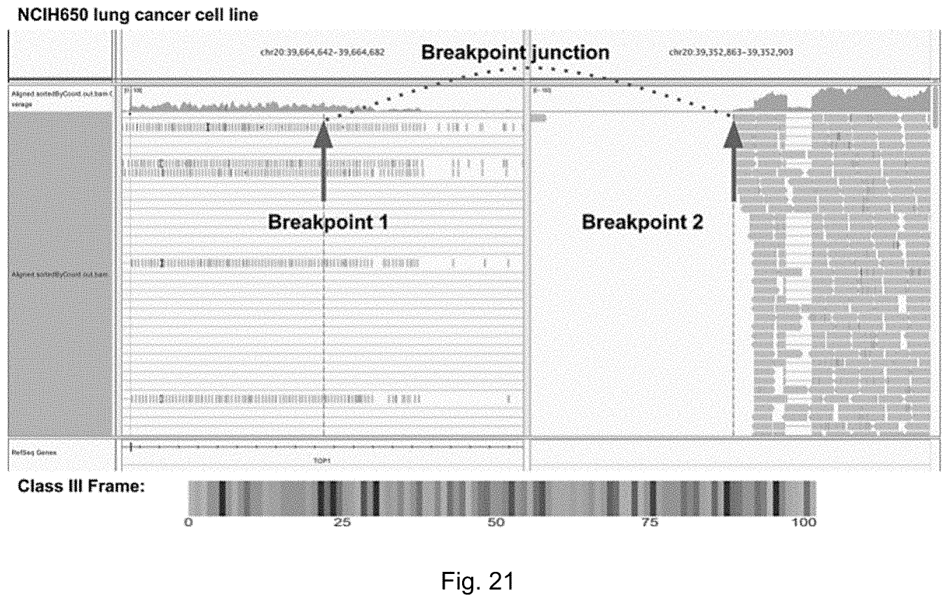

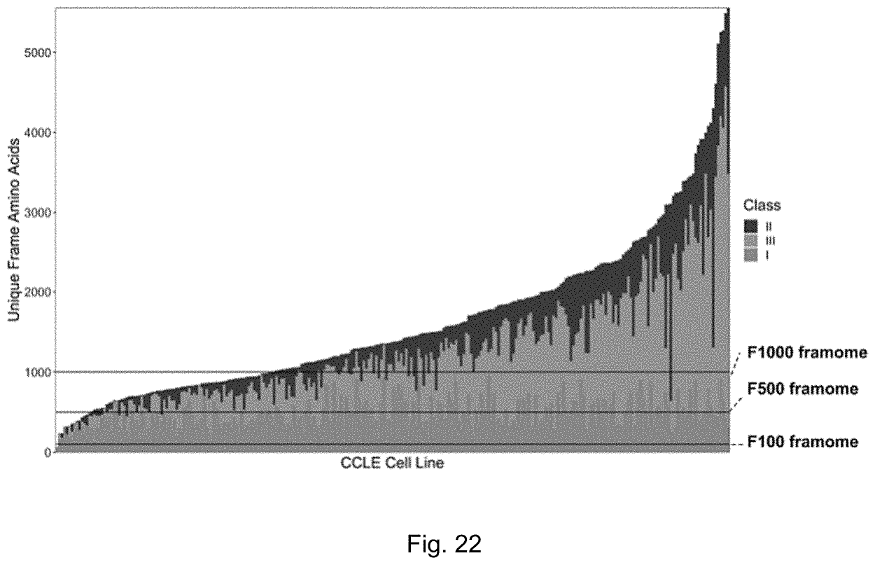

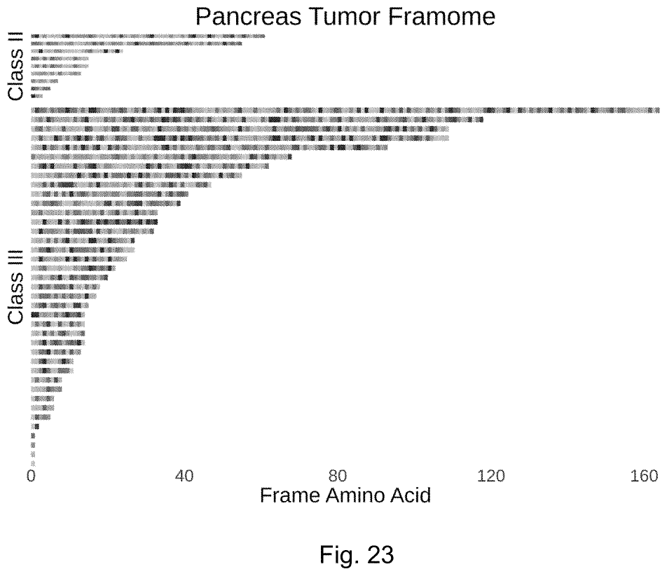

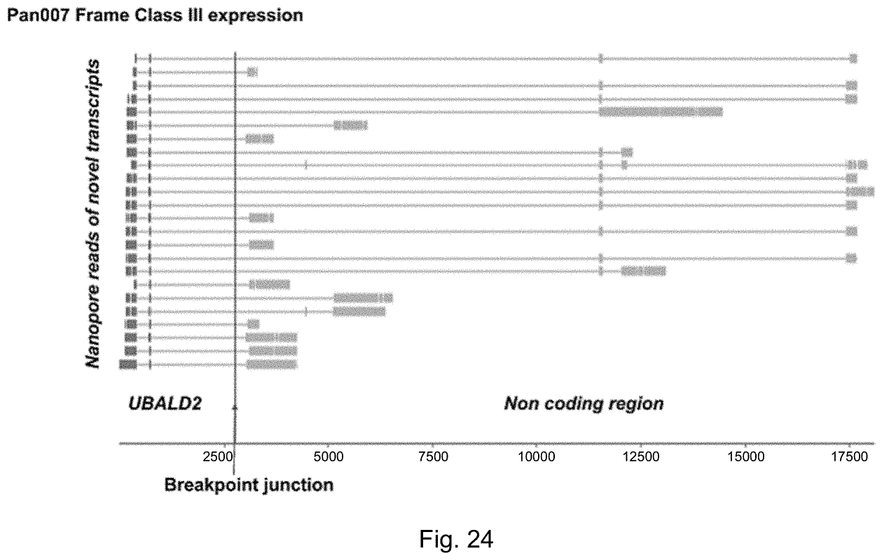

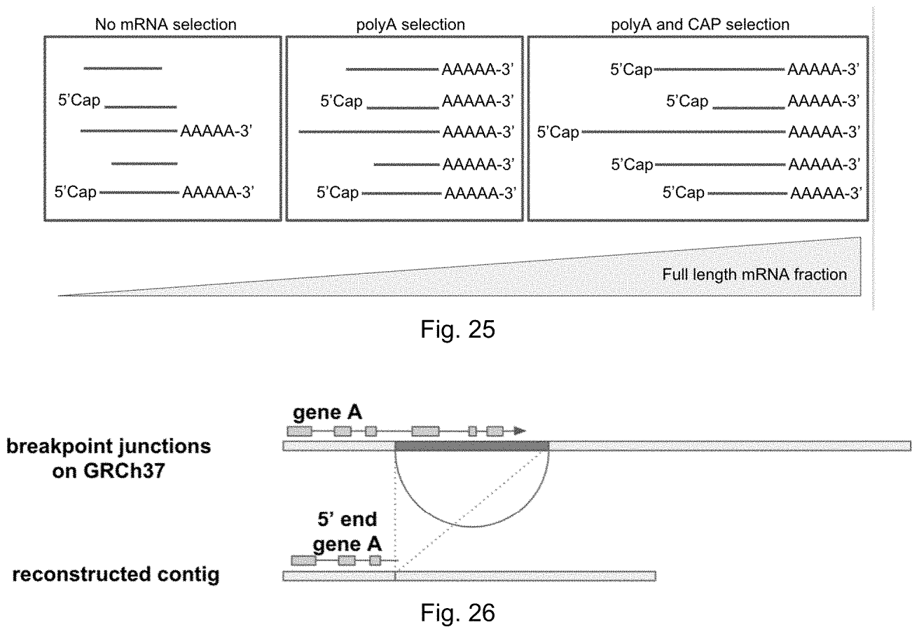

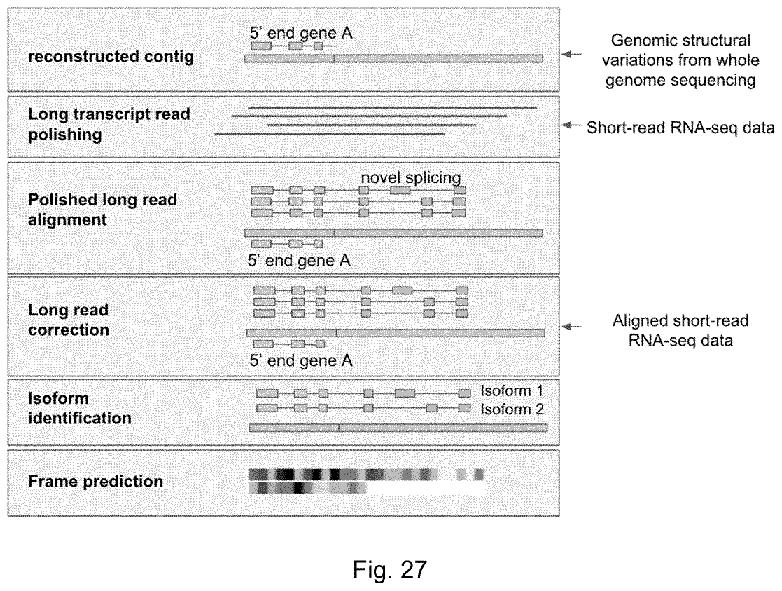

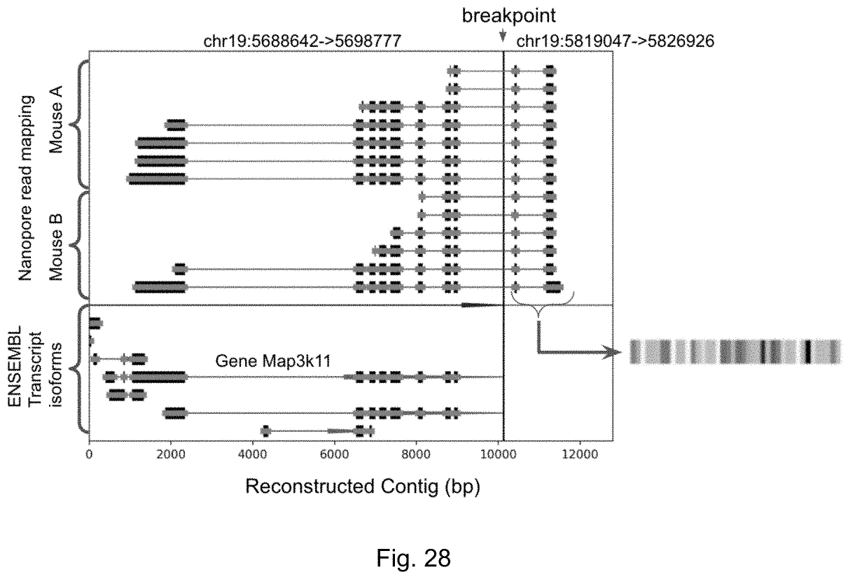

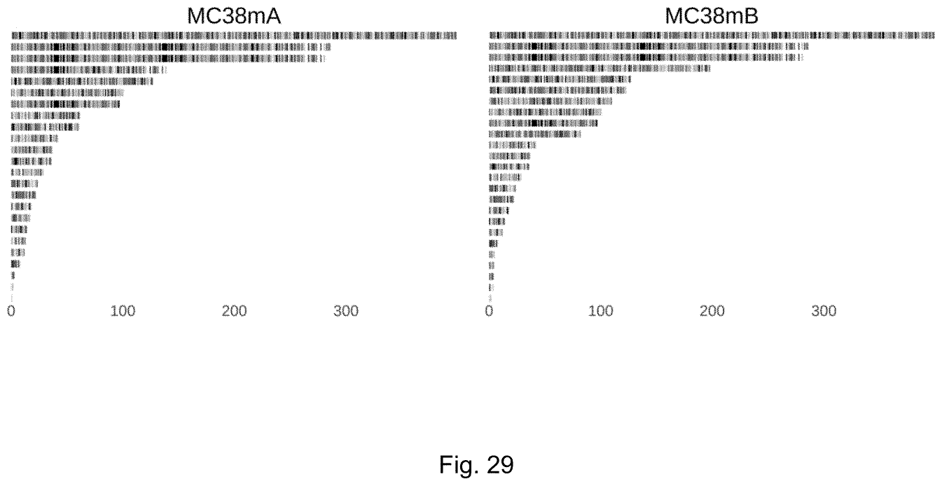

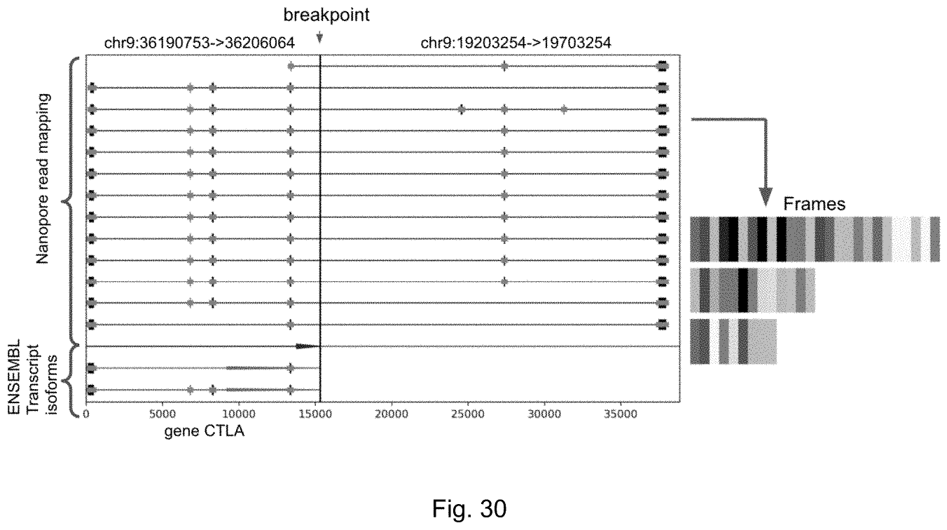

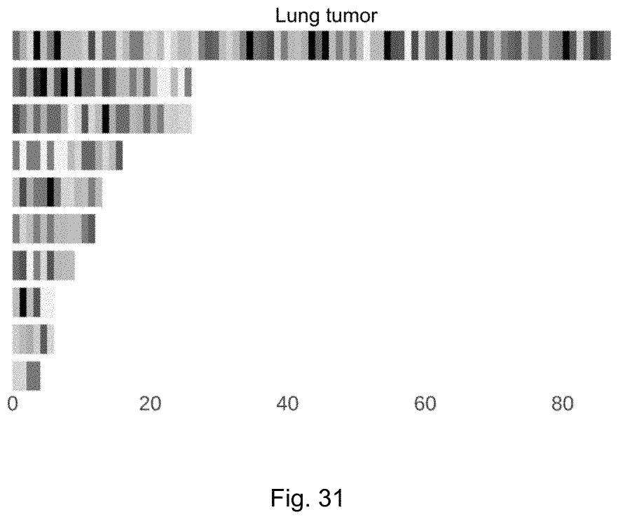

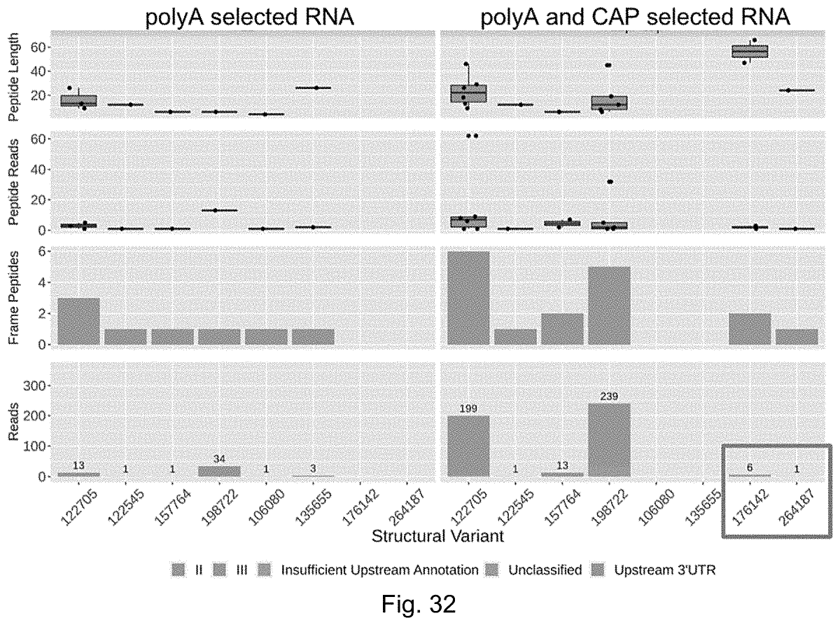

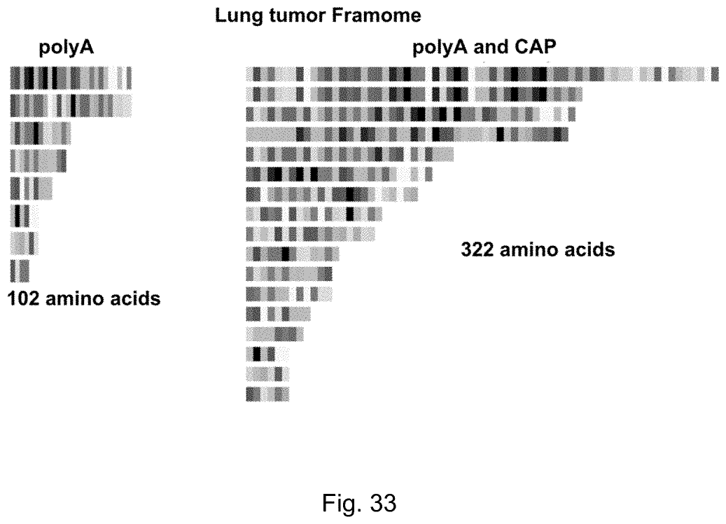

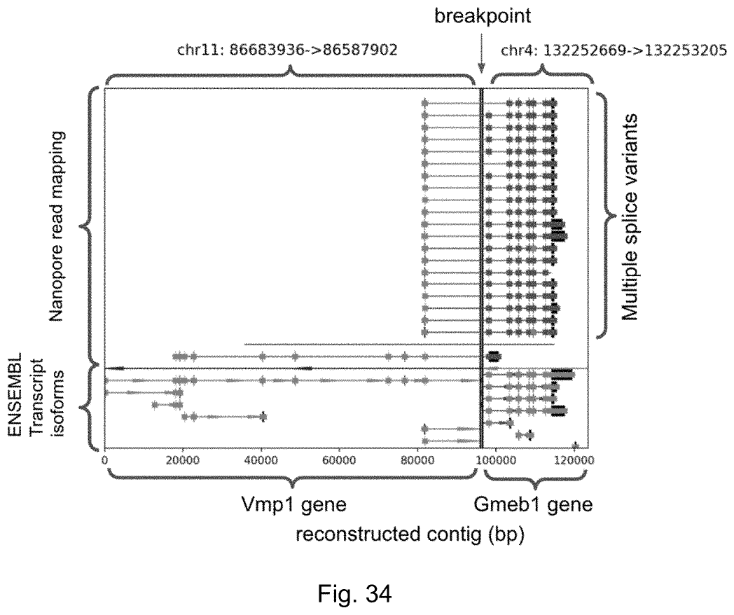

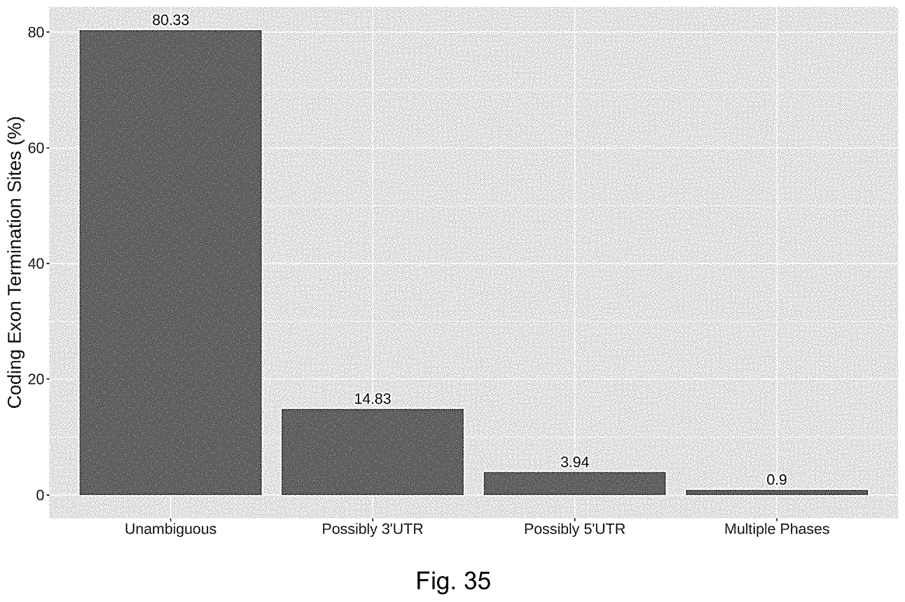

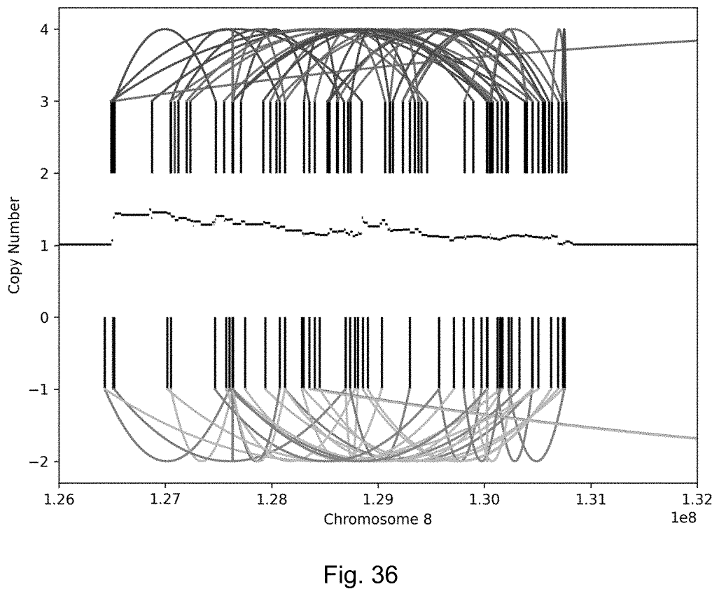

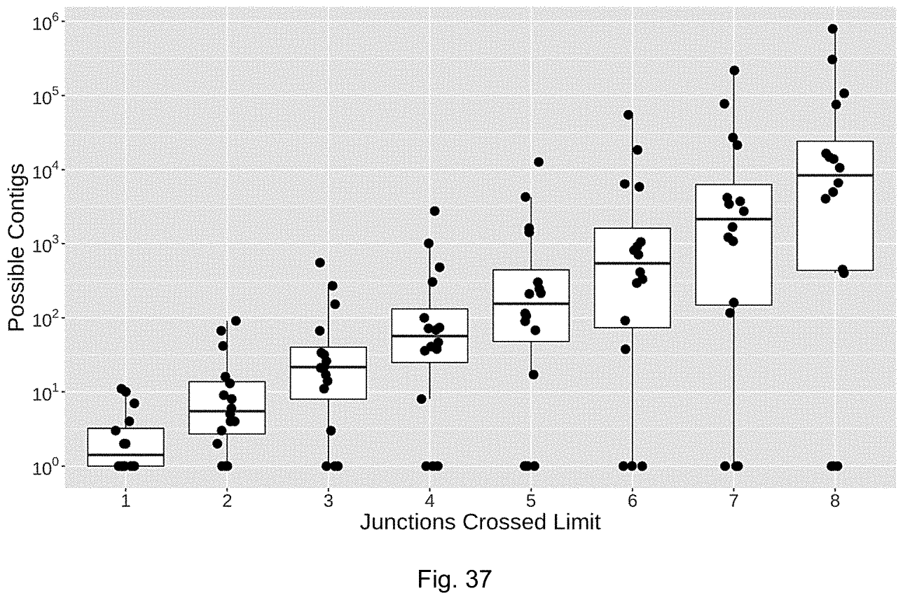

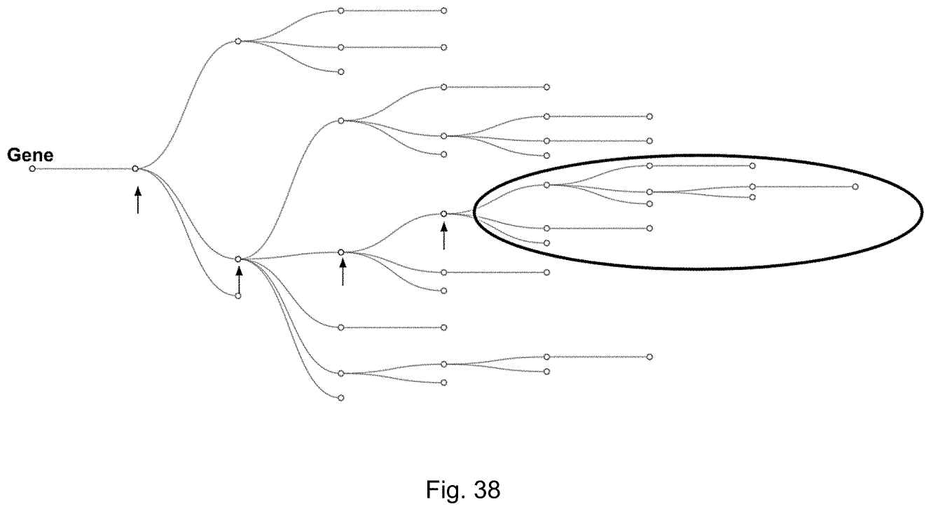

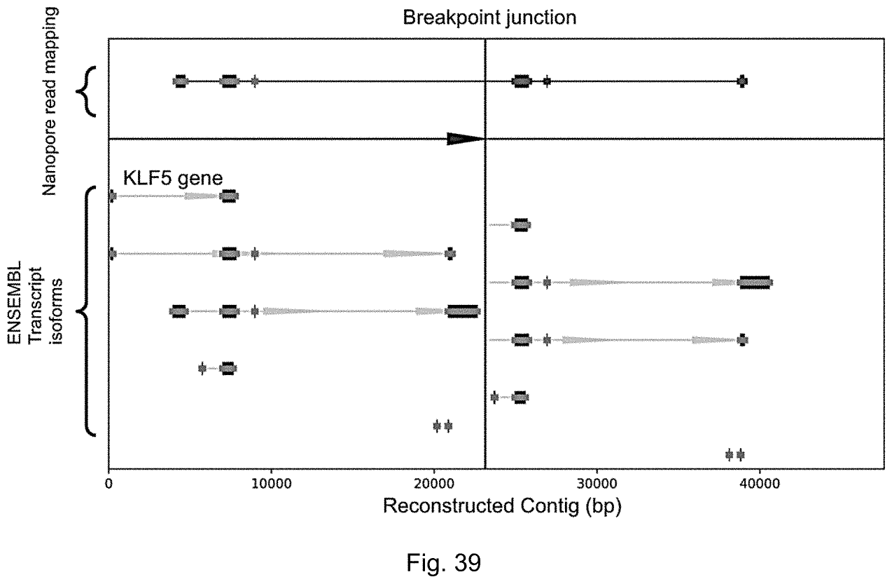

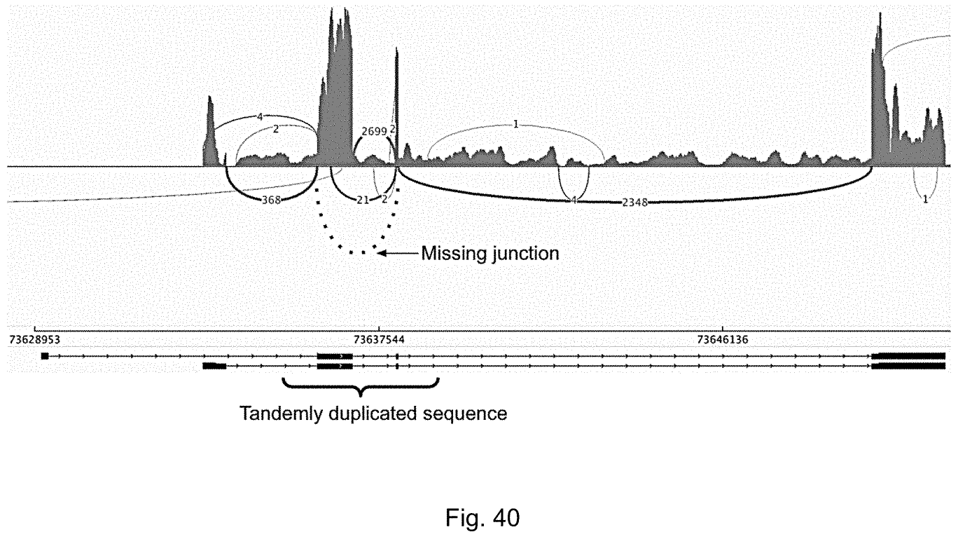

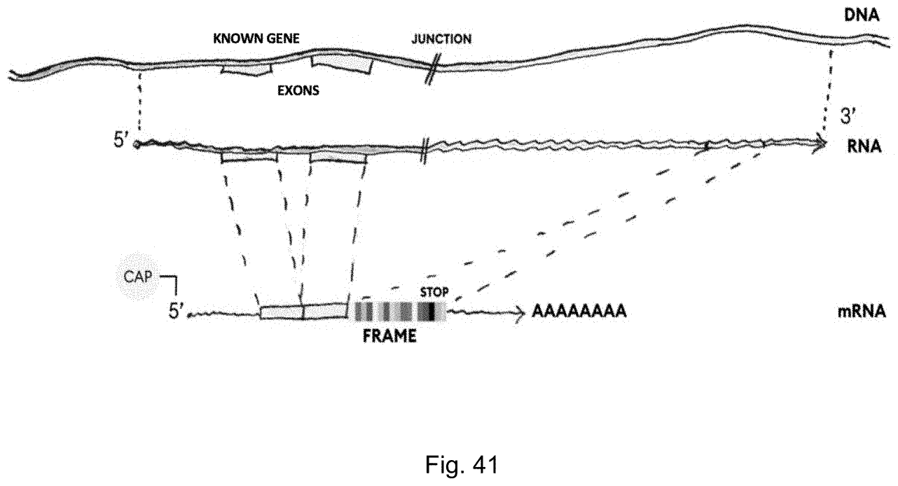

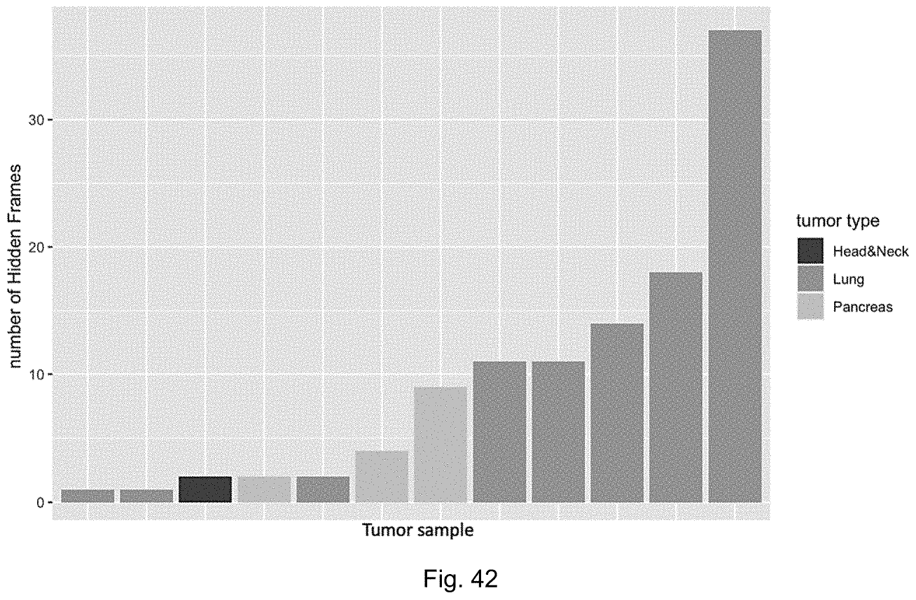

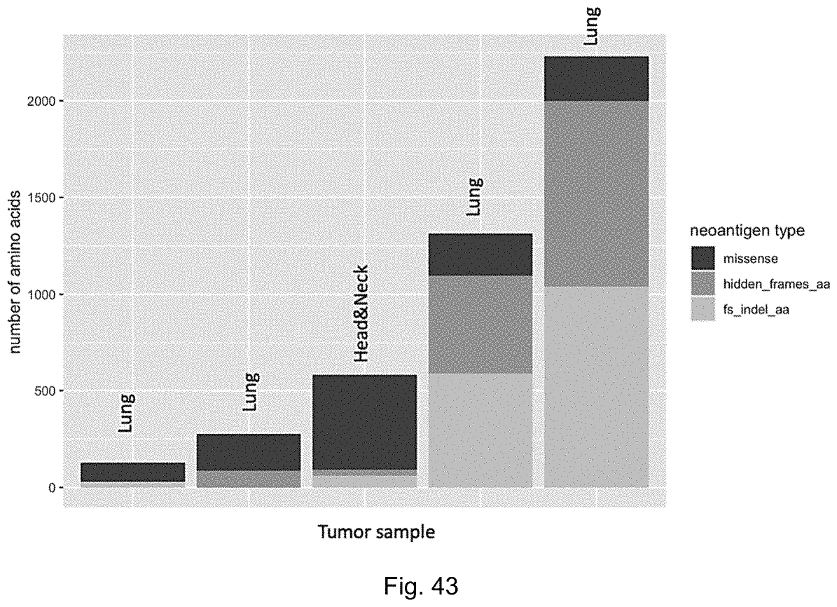

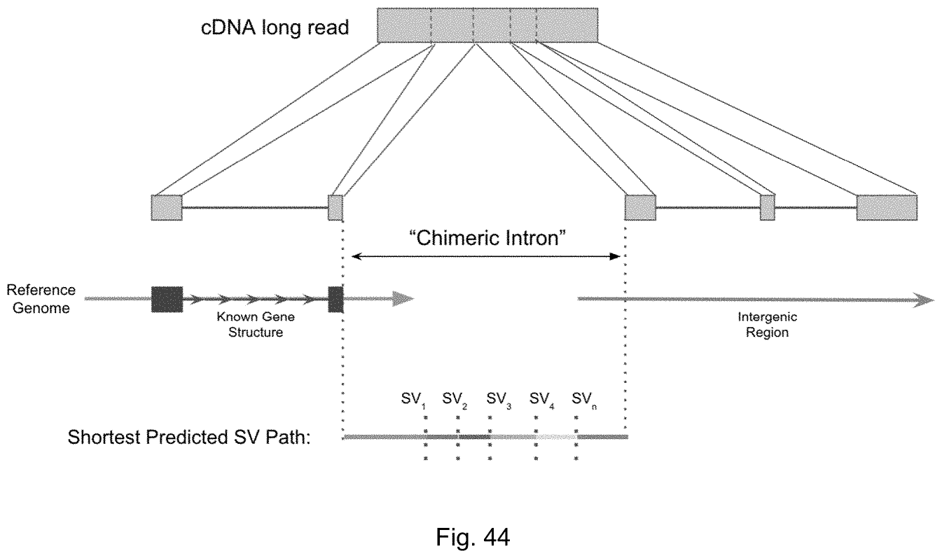

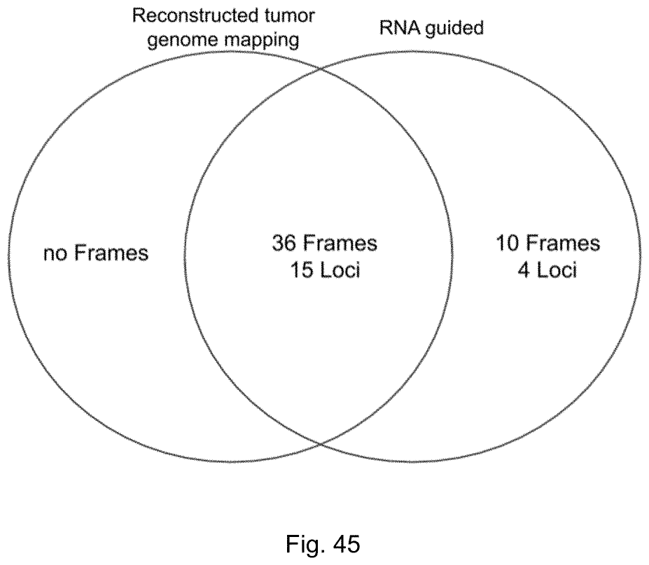

FIG. 10. Numbers of Frames and amino acids for Frames class I, II and III for each of 328 tumor cell lines from the CCLE collection.
10(A). Stacked bar diagram indicating the numbers of class I, II and III Frames, as observed in 328 tumor cell lines from the CCLE project (https://portals.broadinstitute.org/ccle). The Y-axis represent the number of class I, II and III Frames (indicated with different greyscales, see legend). Each vertical bar represents 1 tumor cell line present in the CCLE collection (X-axis). For each Frame class we considered both expressed and non-expressed Frames. For class I and intergenic class II Frames the expression is indicated. The numbers were generated by predicting Frame peptide sequences according to the logic indicated in FIG. 3, and FIGS. 5-8, given the variant calls (indels and translocations) provided by the CCLE portal as input. Frames were only counted if they have a predicted length of at least 1 amino acid.
10(B). Stacked bar diagram indicating the number of novel amino acids encoded by class I, II and III Frames, as observed in 328 tumor cell lines from the CCLE project (https://portals.broadinstitute.org/ccle). The Y-axis represent the number of novel amino acids comprised by class I, II and III Frames (indicated with different greyscales, see legend). Each vertical bar represents 1 tumor cell line present in the CCLE collection (X-axis). For each Frame class we considered both expressed and non-expressed Frames. For class I and intergenic class II Frames the expression is indicated. The numbers were generated by predicting Frame peptide sequences according to the logic indicated in FIG. 3, and FIGS. 5-8, given the variant calls (indels and translocations) provided by the CCLE portal as input. All Frame sizes were considered for determining the amino acid counts. The amino acid counts are restricted to novel amino acids and do not include normal amino acids preceding the frame-shift or rearrangement breakpoint.

FIG. 11. Possible configurations of genes and genomic segments for class II and III Frames. Genomic segments (represented by solid lines with arrow heads) can be joined in any of four possible configurations (often referred to as tail-to-head, head-to-tail, tail-to-tail and head-to-head, or 3′ to 5′, 5′ to 3′, 3′ to 3′, 5′ to 5′, respectively). For class II Frames, genes encoded by both of the two joined genomic segments are fused depending on the gene orientation, i.e. whether the gene is encoded on the + or − strand of the genomic DNA. In case of class III Frames, either only one of the joined genomic segments contains a (part of a) gene, which is always directed towards the breakpoint junction, or, when both of the two genomic segments contain a gene, both genes will have to be in opposite directions, with one of the two genes directed towards the breakpoint junction.

FIG. 12. Cumulative numbers of class I frames for cancers in the TCGA database. The x-axis represents the amount of class I frames (>9 amino acids). The y-axis indicates the fraction of cancer patients with at least the indicated amount of class I frames. The headers of each graph indicate the tumor type as based on TCGA cancer type nomenclature.

FIG. 13. Average length of the class I Framome (in amino acids) for all cancers in TCGA. In this plot, the MSI high tumors were excluded because this distorts the average length per tumor type to the higher end. MSI high was defined in this case as tumors having more than 1000 neo-amino acids encoded as a result of frameshift indels.

FIG. 14. Two examples of Frames that can be selected from a set of Frames encoded by a cancer genome sequence. (i) a long out-of-frame (Frame) sequence of 20 amino acids is resulting from a frameshift mutation. The entire out-of-frame sequence can be used for a cancer vaccine, with or without 1 or more in-frame upstream amino acids from the N-terminal portion of the protein. (ii) a short out-of-frame sequence of 4 amino acids is resulting from a frameshift mutation. The 4 novel amino acids can be combined with 5 preceding in-frame amino acids to form a 9 amino acid long neoepitope that can be used as a cancer vaccine.

FIG. 15. Frame selection for tumor cell lines leads to a set of class I Frames that is optimal for inclusion in a cancer vaccine. Each of the selection steps is depicted on the x-axis. The number of Frames is represented by the y-axis. Left panel indicates MSI-high cell lines and right panel MSI-low cell lines. Similar selection criteria can be applied to class II and III Frames. Total=all Frames; size=Frames with size>9 amino acids; selfmatch=no match to known human protein sequences with match length >7 amino acids; WGS=genomic variant allele frequency >0.2; RNA=expression observed by at least 1 RNAseq read; Cyst=cysteine content <0.1; MHC=match to at least one MHC class I binding epitope.

FIG. 16. Framome of Class I, II and III Frames for lung cancer cell line CHAGOK1 present in the CCLE cell line collection. Frames for each of the three classes were predicted as described for FIG. 10. Both expressed and non-expressed Frames were included and only Frames that are at least 9 amino acids in length are depicted.

FIG. 17. Framome of Class I, II and III Frames for breast cancer cell line EFM192A present in the CCLE cell line collection. Frames for each of the three classes were predicted as described for FIG. 10. Both expressed and non-expressed Frames were included and only Frames that are at least 9 amino acids in length are depicted.

FIG. 18. Framome of Class I, II and III Frames for oesophagus cancer cell line KYSE520 present in the CCLE cell line collection. Frames for each of the three classes were predicted as described for FIG. 10. Both expressed and non-expressed Frames were included and only Frames that are at least 9 amino acids in length are depicted.

FIG. 19. Example of the use of Oxford Nanopore long read sequencing for the determination of transcript structure of the mouse Pdxk gene (encoded on the − strand of mouse chromosome 10). The Pdxk gene contains a frameshift deletion in the mouse tumor cell line MC38. Individual Nanopore reads were mapped to the mouse reference genome MM10 and each read is depicted as a separate horizontal line, with mapped regions indicated as a thicker part of the line. The grey reads contain the indel mutation while the black reads are derived from the wildtype (normal) allele. The Pdxk transcript structure is indicated below the Nanopore reads. Transcript structure was obtained from the Ensembl database for mouse genome MM10. The position of the indel is indicated with a black vertical line. Note that some of the transcripts are shorter at the 3′-end (the left part of each read) than others. Furthermore, exons are skipped for some of the transcripts. Taking full length transcript structures into account is essential for Frame peptide prediction.

FIG. 20. Example of expression of class III Frames in breast cancer cell line HCC1954. Available tumor cell line short-read RNA sequencing data were analysed using the Integrative Genomic Viewer (IGV) [https://software.broadinstitute.org/software/igvl]. One breakpoint is in the OXR1 gene and the other breakpoint is in a genomic segment without any know protein coding gene. The fusion of the 5′ part of the OXR1 gene leads to read through transcription into the genomic segment following breakpoint 2. An expressed Class III Frame sequence is resulting from this genomic rearrangement.

FIG. 21. Example of expression of class III Frames in lung cancer cell line NCIH650. Available tumor cell line short-read RNA sequencing data were analysed using the Integrative Genomic Viewer (IGV) [https://software.broadinstitute.org/software/igv/]. One breakpoint (left) is in the TOP1 gene and the other breakpoint is in a genomic segment without any know protein coding gene. The fusion of the 5′ part of the TOP1 gene leads to read through transcription into the genomic segment following breakpoint 2. Note that the expression following the second breakpoint (right side) involves cryptic splicing. An expressed Class III Frame sequence is resulting from this genomic rearrangement, depicted at the bottom of the figure with different greyscales for each amino acid.

FIG. 22. Description of Framome vaccine gradations. An F100 framome vaccine represents at least 100 novel amino acids comprising all (expressed) Frames in a cancer genome that are selected for inclusion in a Framome cancer vaccine. An F500 Framome vaccine represent at least 500 novel amino acids comprising all (expressed) Frames in a cancer genome that are selected for inclusion in a Framome cancer vaccine. An F1000 Framome vaccine represent at least 500 novel amino acids comprising all (expressed) Frames in a cancer genome that are selected for inclusion in a Framome cancer vaccine. The percentage of tumor samples covered by an F100, F500 and F1000 Framome vaccine is respectively, 99.6%, 94.8%, 68.1%. The data are based on human tumor cell lines for which we predicted class I, II and III Frames from genome sequencing data. Data are only shown for MSI-L tumor cell lines.

FIG. 23. Framome of pancreas tumor. Based on whole genome sequencing of a pancreas tumor sample and corresponding normal sample, we identified a set of class II and class III Frames, resulting from genomic rearrangements in the tumor genome. Class I Frames were not identified. This pancreas tumor Framome covers 1502 potential newly encoded amino acids.

FIG. 24. Example of an expressed classIII Frame in a pancreas tumor sample, covering part of the UBALD2 gene and a noncoding genomic region. Data are derived from Nanopore long read cDNA sequencing of pancreas mRNA (following poly-A selection). Thus, the reported novel transcripts are represented in the tumor as translatable mRNAs encoding novel Frame neoantigens.

FIG. 25. Selection of full-length mRNAs based on 3′-poly-(A) and 5′-CAP selection. Full-length mRNAs containing entire open reading frames, are optimally obtained using RNA-selection and/or sequencing methods that specifically targeted RNA molecules with said 5′-CAP and 3′-poly-(A) tail.

FIG. 26. Schematic overview of local genome reconstruction informed by somatic structural genomic rearrangement breakpoint junctions. A segment from the normal human reference genome (e.g. GRCh37 or GRCh38 or the like) has been deleted in a specific tumor. The genome reconstruction involves the generation of a contig that lacks the deleted segment. This is a simplified example and in practice much more complex rearrangements occur with neighbouring breakpoint junctions leading to complex local genome configurations.

FIG. 27. Possible workflow for identification of Frame neoantigens from a combination of short-read and long-read RNA sequencing and whole genome sequencing using short- and/or long sequencing reads. The process starts with the generation of contigs matching the likely genome configuration occurring in the tumor sample, as based on the identification of somatic genomic structural variation breakpoint-junctions. In a second step, long transcript sequencing reads are generated, which may be additionally polished with accurate short-read RNA sequencing reads. Subsequently, the (polished) long transcript sequencing reads are mapped (aligned) to the reconstructed contig(s), to identify the splice-structure of the transcripts across the breakpoint-junction(s) in the reconstructed contig(s). The long-read transcript alignments are grouped based on the splicing patterns to identify all possible transcript isoforms. Finally, each of the unique transcript isoforms is translated to a peptide sequence and the novel portion of the peptide sequence, encoded by (novel) exons downstream of breakpoint-junction, is selected for design of a vaccine or immunotherapy treatment.

FIG. 28. Mapping of corrected long-read Nanopore cDNA sequencing data to a reconstructed tumor-specific contig for mouse tumor cell line MC38. Data were obtained and analyzed as described in example 1. The reconstructed contig consists of two parts of mouse chromosome 19. One region (chr19:5688642->5698777) contains mouse gene Map3k11, which has multiple known isoforms, as annotated in the Ensembl genome database (ensembl.org). The second region (chr19:5819047->5826926) contains novel exons resulting from novel splicing, which result in a protein product that is unique to the mouse MC38 tumor.

FIG. 29. Framome of mouse tumor cell line MC38, as derived from the experiments described in example 1. Mouse Frame prediction was performed for two separate sequencing datasets from mouse MC38 tumors, derived from two mice (MC38mA, MC38mB). Frames (novel open reading frames) are indicated as horizontal bars with alternating amino acids (different grey shading).

FIG. 30. Mapping of long-read Nanopore cDNA sequencing data to a reconstructed tumor genome for a lung tumor. Data were obtained and analyzed as described in example 2. The reconstructed contig consists of two parts of human chromosome 9. One region (chr9:36190753→36206064) contains human gene CTLA, which has multiple known isoforms, as annotated in the Ensembl genome database (ensembl.org). The second region (chr9:19203254→19703254) contains novel exons resulting from novel splicing, leading to multiple novel transcript isoforms. The transcript isoforms result in three different Frame protein products that are unique to the lung tumor.

FIG. 31. Framome of lung tumor, as derived based on the methods explained in Example 9. Frames are indicated as horizontal bars with alternating amino acids (different grey shading).

FIG. 32. Analysis of Frames in a lung tumor based on RNA sequencing of polyadenylated mRNAs (left panels) and RNA sequencing of polyadenylated and Cap selected mRNAs (right panels). The square in the lower right corner indicates two novel Frames that were only found based on the Cap plus polyadenylated mRNA workflow.

FIG. 33. Framomes of a lung tumor detected based on long-read RNA sequencing data derived from poly-A selected mRNAs (left panel) and poly-A and Cap selected mRNAs (right panel). Many more Frames are detected when applying a poly-A and 5′-CAP selection procedure, compared to a poly-A only selection procedure.

FIG. 34. Example of an out-of-frame gene fusion leading to a Frame neoantigen. Long read RNA sequencing data were mapped to a reconstructed contig containing a somatic breakpoint-junction identified in mouse tumor MC38. The mapping of the long-read RNA data identifies the transcript isoforms of the Vmp1 and Gmeb1 genes which are involved in the chimeric transcripts. Multiple chimeric transcripts (splice isoforms) occurred, which are fully resolved by the long-read RNA sequencing data.

FIG. 35. Exon exit ambiguity in the human genome. The ENSEMBL coding exon end positions were annotated according to the exons sharing the loci. For almost 20% of the sites multiple annotations exist, which would hamper the unambiguous prediction of downstream Frame sequences.

FIG. 36. Example of a complex genomic rearrangement in an Acute Myeloid Leukemia sample. The copy number is visualized as horizontal lines/marks deviating along the y-axis. The somatic genomic breakpoint-junctions are visualized as arcs above and below the copy number profile.

FIG. 37. Boxplots indicating the number of possible contigs given 1, 2, or more (up to 8) crossed breakpoint-junctions for a complex genomic rearrangement in an Acute Myeloid Leukemia from FIG. 36. The number of possible contigs increases exponentially with a larger number of breakpoint-junctions for this complex rearrangement. Each dot represents a single gene, hit by one or more breakpoint-junctions. The y-axis represents the maximum number of crossed breakpoint-junctions.

FIG. 38. Reducing the complexity of reconstruction of tumor-specific reference sequences using long-read DNA sequencing. Each node indicates a breakpoint-junction. The four breakpoint-junctions indicated by arrows are all connected using long Nanopore sequencing reads. These connections can be traversed to reach only one branch in the tree, that contains only a limited number of possible remaining genomic configurations.

FIG. 39. Example of intragenic tandem duplication in the KLF5 gene in a tumor genome. Long Nanopore (cDNA) transcript reads were mapped to a reconstructed contig containing the tandemly duplicated sequence. The novel transcript sequence discovered by the Nanopore reads involves tandemly duplicated exons which encode a novel Frame sequence. The tandemly duplicated exonic structure could only be resolved by aligning the long-read Nanopore cDNA reads to a tumor-specific genomic contig containing the tandemly duplicated segments.

FIG. 40. Sashimi plot of splice junctions in the KLF5 gene based on short read RNA sequencing data. The short-read RNA sequencing data were mapped to the normal GRCh37 reference (which does not contain the tandemly duplicated sequences found in the tumor genome). The KLF5 gene contains an intragenic tandem duplication in this tumor sample. The short-read RNA junctions do not identify this junction, when aligned to the normal GRCh37 reference. However, the junction is found when mapping long-read Nanopore RNA sequencing reads to a reconstructed tumor-specific contig containing the tandemly duplicated sequence, as shown in FIG. 39.

FIG. 41. Schematic of tumor neoantigens resulting from rearrangements plus splicing, which are referred to herein as class III Frames or hidden Frames.

FIG. 42. Numbers of hidden Frame (class III) neoantigens in pancreas, lung and head & neck cancers. Hidden Frame neoantigens were identified based on a sequencing of full-length capped and polyadenylated mRNAs and mapping of sequencing reads for those mRNAs to the human reference genome.

FIG. 43. Comparison of Frames, hidden Frame neoantigens and missense neoantigens in several human tumor samples. The number of amino acids is determined as the sum of the length of the novel Frame neopeptide sequences resulting from hidden Frames (i.e., class III) (hidden_frames_aa) and class I and II Frames (fs_indel_aa). For each missense mutation one amino acid is counted.

FIG. 44. Schematic overview of detection of hidden Frame (class III) neoantigens by long-read cDNA mapping and subsequent confirmation of cDNA mapping in tumor genomic DNA.

FIG. 45. Detection of hidden Frame neoantigens by long-read cDNA mapping to (i) a reconstructed tumor genome as defined by genomic structural variation breakpoints (left) and (ii) a reconstructed tumor genome as defined by mapping of long RNA (cDNA) to the human reference genome. In both cases, short- and long-cDNA reads are aligned to the reconstructed tumor-specific reference sequences to identify Frames. More hidden Frame neoantigens were identified using an RNA-directed reconstruction of the tumor-specific reference genome.

### DETAILED DESCRIPTION OF THE DISCLOSED EMBODIMENTS

Approximately 95% of all coding mutations in tumor genomes are single nucleotide variations (excluding synonymous changes). However, the present disclosure demonstrates that the majority of new amino acids encoded by the tumor genome are the result of frameshift mutations or genomic rearrangements, which result in neo-open reading frames. Depending on tumor type, more than three-fourths of the neoantigen amino acids in tumors are the result of indels and genomic rearrangements as a source of neoantigens. Although such mutations are on average only a few percent of the coding mutational load of a tumor, they can lead to long stretches of new amino acids.

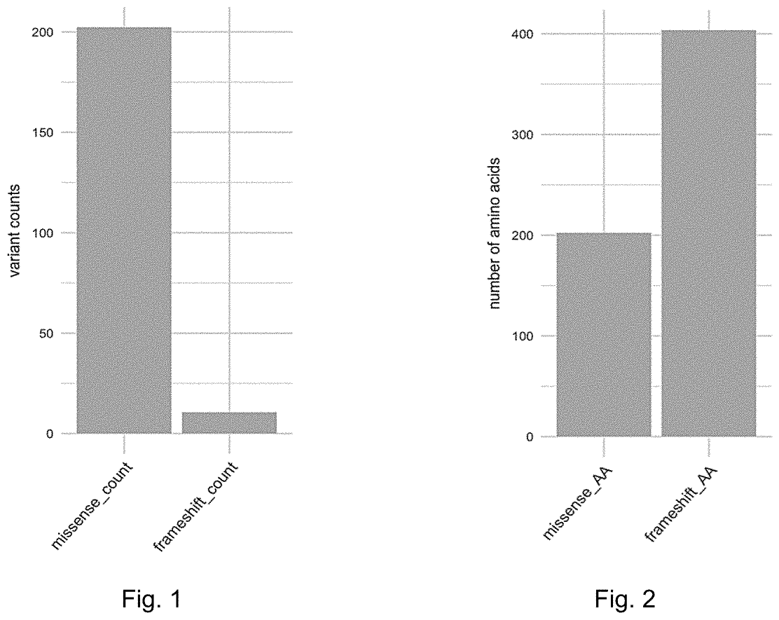

Carcinogenesis is a numbers game, with an unfortunate combination of driver mutations turning a healthy body cell into a tumor cell. The immune response to neoantigens in the tumor is similarly a numbers game (in which apparently some cell lineages manage to escape and develop into full blown cancer). The choice of the best vaccine based on discovery of neoantigens in the DNA sequence of a tumor is also a numbers game. In FIG. 1 it is shown that on average 95% of all coding mutations in the ORFeome of tumors (excluding synonymous variations) are missense SNVs (Single Nucleotide Variants), as based on the tumor mutation reports available for the TCGA database.

Much of the research in recent years has focused on prediction (either in silico or by experimental analysis) which of these many mutations would make for the best neoantigen to use as a vaccine Schumacher, T. N., Scheper, W. & Kvistborg, P. Cancer Neoantigens. Annu. Rev. Immunol. 37, 173-200 (2019). On average (but widely differing per tumor type) a tumor ORFeome contains 200 missense mutations (Priestley, P. et al. Pan-cancer whole genome analyses of metastatic solid tumors. bioRxiv 415133 (2019). doi:10.1101/415133), and the practical limit of the number of peptide vaccines that can be applied to any patient has been set anywhere between 5 and 20, so that at max a few percent of the neoantigens caused by missense mutations can be used for vaccination. Therefore, indeed the choice of the “best” SNVs is indeed crucial. In this choice it is usually considered that the peptide containing the SNV-neoantigen needs to be presented by the MHC, so that prediction of the presentation by the MHC-type of the patient is essential. For vaccine technologies other than peptides, such as DNA or RNA encoded vaccines, the number of SNVs to be included in a vaccine may be higher than 5-20, but practical limitations still preclude the inclusion of all of them. Similarly, for cellular immunotherapies, such as therapies making use of T-cells with engineered T-cell receptors (TCR) sequences, the number of neoantigens that can be targeted is also limited.

In contrast to the detection of SNVs, the methods disclosed herein identify somatic changes that result in novel open reading frames, also referred to as neoORFs or ‘Frames’. These somatic changes are described further herein. While not wishing to be bound by theory, the inventors propose that cancer therapies (such as vaccines) with the best chance of success will be those based on a large part of a tumor's antigenicity. In contrast to current approaches which look for the “best” neoantigens to treat a tumor, the present disclosure provides methods which can identify a large part of the tumor neoantigenicity.

We have found that a surprisingly large amount of antigenicity results from complex rearrangement and splicing events such as those depicted in FIG. 8B, FIG. 11, FIG. 20, FIG. 21, FIG. 24, FIG. 28, FIG. 30 and FIG. 41. Such neoantigens resulting from (complex) genomic rearrangements and mRNA splicing, are herein referred to as Hidden Frame Neoantigens (or Hidden Frames, or class III Frames). Since close to 99% of the human genome is non-coding DNA, the vast majority of DNA rearrangements (i.e., structural variants) occur in non-coding DNA. When genomic sequences that encode for known proteins are fused via DNA rearrangements, the resulting mRNA product (and corresponding protein sequence) can in many cases be predicted on the basis of the known splice site donors and acceptors (see FIG. 5 and FIG. 6). However, when a DNA rearrangement (i.e., structural variant) occurs that fuses a genomic sequence encoding a gene with non-coding DNA, the resulting mRNA product cannot be predicted since there are no known splice site donors and acceptors in the non-coding DNA.

While not wishing to be bound by theory, the present disclosure proposes that Hidden Frames (class III Frames) are the product of two events. The first event is a structural variation that fuses the non-coding or coding region of the coding strand of a known gene (most often an intronic sequence, but exonic or other sequence is also possible) to another (or multiple other) segment(s) in the tumor genome (e.g., non-coding DNA or the noncoding strand of a gene). The second event is one or more splicing events that occur during the processing of the primary transcript (that crosses the structural variation junction) into mature mRNA. These splicing events cannot be predicted in the current state of the art based solely on the DNA sequence. The disclosure provides the sequencing of mature mRNA in order to combine the information regarding the structural variant with the sequence of the mature mRNA.

As used herein, the term “open reading frame” or ORF refers to a nucleic acid sequence comprising or encoding a continuous stretch of codons. As used herein the term “neoORF” refers to a tumor-specific open reading frame (i.e., novel open reading frame) arising from a frame shift mutation or DNA rearrangement. Such neoORFs are not present in the germline and/or healthy cells of an individual. Peptides arising from such neoORFs are referred to herein as neoantigens or ‘Frames’. The methods described herein have been developed, at least in part, in order to maximize the number of neoantigen amino acids identified from the tumor of an individual. As used herein, the term ‘Framome’ refers to all, or essentially all, of the neoORFs that result from somatic genetic changes as described herein (indels and genomic rearrangements) that can be identified in a tumor sample using whole genome sequencing.

There are two major advantages of using the Framome as therapeutic anti-cancer vaccines or other forms of immunotherapy. Firstly, Frames are presumed to be the most antigenic neoantigens encoded by tumor genomes as compared to SNV-antigen⁷. If the potential antigenicity of a tumor were to be expressed as the number of newly encoded amino acids, the Framome covers much, if not the majority of all antigenicity (FIG. 2, FIG. 9, FIG. 10), and thus largely takes the selection process for the best possible neoantigens out of vaccine development.

Secondly, Frames have an additional advantage over SNV-antigens in regards to HLA-restriction. Small peptides containing a single amino acid change will be presented within the MHC with only few options for a productive presentation, and thus the precise fit of the chosen peptide within the MHC of the specific HLA type of the patient is a point of serious attention¹. For long viral antigens it has long been concluded that such concern about HLA-matching is of less importance, since the long and entirely foreign (non-self) sequence will be degraded by the proteasome in so many different ways that along the full length of the neoantigen there will always be stretches that match and are thus productive antigens. This also applies to Frames, which are in this respect no different than e.g. the HPV16 and HPV-17 antigens encoded by the Human Papilloma Virus, and which are used successfully for anti-tumor vaccination (Massarelli et al. JAMA Oncol 2019 5:67-73).

While cancer specific frameshift mutations have previously been described, one objection of the disclosure is to identify a larger source of potential neoantigens which includes Frames resulting from the structural genomic variations described further herein. In particular, class III Frames (as described herein and also indicated as Hidden Frames) represent a novel source of neoantigens.

In one aspect, the disclosure provides a method for identifying candidate neoantigen sequences. The neoantigen sequences are identified from a tumor sample of an individual afflicted with cancer. As described further herein, such neoantigens may be used to prepare a vaccine for the treatment of cancer.

As used herein the term “sequence” can refer to a peptide sequence, DNA sequence or RNA sequence. The term “sequence” will be understood by the skilled person to mean either or any of these and will be clear in the context provided. For example, when comparing sequences to identify a match, the comparison may be between DNA sequences, RNA sequences or peptide sequences, but also between DNA sequences and peptide sequences. In the latter case the skilled person is capable of first converting such DNA sequence or such peptide sequence into, respectively, a peptide sequence and a DNA sequence in order to make the comparison and to identify the match. As is clear to a skilled person, when sequences are obtained from the genome or exome, the DNA sequences are preferably converted to the predicted peptide sequences. In this way, neo open reading frame peptides are identified. The neoantigens can include a polypeptide sequence or a nucleotide sequence encoding said polypeptide sequence.

The methods comprise identifying somatic genomic changes in nucleic acid sequences from at least one tumor sample from the individual, wherein the somatic genomic changes result in new open reading frames.

As used herein the term “sample” can include a single cell or multiple cells or fragments of cells or an aliquot of body fluid, taken from an individual, by means including venipuncture, excretion, ejaculation, massage, biopsy, needle aspirate, lavage sample, scraping, surgical incision, or intervention or other means known in the art. The nucleic acid for sequencing is preferably obtained by taking a sample from a tumor of the patient. The skilled person knowns how to obtain samples from a tumor of a patient and depending on the nature, for example location or size, of the tumor. Preferably the sample is obtained from the patient by biopsy or resection. The sample is obtained in such manner that it allows for sequencing of the genetic material obtained therein.

The term ‘individual’ includes mammals, both humans and non-humans and includes but is not limited to humans, non-human primates, canines, felines, murines, bovines, equines, and porcines. Preferably, the mammal is a human.

A skilled person can readily identify genomic changes in a sequence. Preferably, whole genome sequencing is used. While partial sequencing or targeted sequenced is often used on tumor tissue, such methods primarily identify Single Nucleotide Variants (SNVs), or other small genetic variations present in (protein) coding sequences of the genome. In order to determine whether such genomic changes are somatic, the sequences obtained from the tumor sample can be compared to sequences from non-tumor tissue of the patient, e.g., blood. The comparison of tumor sequences and sequences from non-tumor tissue are often compared via mapping of the sequences to a human reference genome, as is known by a person skilled in the art.

Frameshift Mutations

The first class of mutations refers to intragenic frameshift mutations in polypeptide encoding sequences, wherein the mutation results in a change of the reading frame of said polypeptide encoding sequence. Class I Frames result from insertions and deletions within coding exons of a single gene. As is well-known to a skilled person, a “frame shift mutation” is a mutation causing a change in the frame of the protein, for example as the consequence of an insertion or deletion mutation (other than insertion or deletion of 3 nucleotides, or multitudes thereof). Such frameshift mutations result in new amino acid sequences in the C-terminal part of the protein. These new amino acid sequences (encoded by the new open reading frame) generally do not exist in the absence of the frameshift mutation and thus only exist in cells having the mutation (e.g., in tumor cells and pre-malignant progenitor cells).

Frameshift mutations can be identified based on the exome from the tumor, although whole genome sequencing may be preferred. Expression of relevant Frames resulting from frameshift mutations can be determined by RNA sequencing.

Structural Variations (SV)

A second type of mutation that leads to novel Frames are DNA rearrangements, in particular structural variations. Structural variations are DNA rearrangements, which encompass at least 50 bp although such variations are normally around 1 kb or larger in size. SVs include, e.g., deletions, duplications, insertions, inversions, and translocations. See for a review Mahmoud et al. Genome Biology 2019 20:246. While neoantigens caused by SVs are relevant in the majority of tumors, this source of antigenicity is especially relevant in cancers having complex chromosome rearrangements such as chromothripsis, chromoplexy and chromoanasynthesis.

SVs may result in DNA gain (e.g., copy number variations, such as tandem duplications), DNA loss (e.g., deletions which may disrupt gene function), as well as balanced rearrangements that do not involve loss or gain of chromosomal sequence (e.g. inversions, reciprocal translocations). Each of the possible SV types may possibly lead to new open reading frames. Such rearrangements may lead to Frame neoantigens, referred to herein as class II) and class III) Frames.

While not wishing to be bound by theory, the inventors propose that a large part of tumor antigenicity derives from novel open reading frames caused by DNA rearrangements. In particular, the disclosure provides methods for identifying neoantigens that are the result of DNA rearrangements, wherein the rearrangement results in the fusion of at least part of the coding strand of a first gene to another sequence in the genome. In particular, the rearrangement results in the fusion of the 5′ portion of a gene to another sequence in the genome, such that the neoantigen is in frame with the start of the known gene in the 5′ fusion partner within the mature mRNA. Such rearrangements may lead to Frame neoantigens, referred to in some embodiments herein as class II) and class III) Frames.

We have found that in many cases during transcription of a ‘proper’ gene that spans a genomic breakpoint-junction which connects the gene to another piece of the genome, the transcription machinery will seek and find a preferred place for transcription termination and polyadenylation of the RNA and the splicing machinery will seek and find splice sites. The result is a fully processed and translatable mRNA, complete with 5′-CAP and poly-(A)-tail. In our results, we observe that there is often either one or only a few dominant mRNA variants that emerge from the process of transcription across somatic genomic breakpoint-junctions and RNA-processing (FIG. 28, FIG. 30). These variants result in new open frames and are a large source of tumor antigenicity.

Class II)

One type of structural variant refers to DNA rearrangements resulting in new junctions of DNA sequences, wherein the rearrangement results in the fusion of at least part of the coding strand of a first gene to at least part of the coding strand of a second gene. Alternatively, the rearrangement results in an intragenic rearrangement, such as an intragenic deletion or (tandem) duplication, thereby creating an intra-genic fusion, between the upstream (5′) part of a gene and the downstream (3′) part (in particular the poly-(A) signal). In particular, the DNA rearrangement results in a change of the reading frame of a polypeptide encoding sequence. The present methods identify somatic changes resulting in new open reading frames. Such variants are also referred to herein as “class II” mutations. Particularly, these DNA rearrangements results in a change of the reading frame of a polypeptide encoding sequence.

In some embodiments, class II) mutations result in the fusion of at least part of the coding strand of a first gene to at least part of the coding strand of a second gene (i.e., intergenic genomic rearrangement). In regards to class II mutations that result in the fusion of at least part of the coding strand of a first gene to at least part of the coding strand of a second gene, the reading frames of the first and second gene are different at the position of the junction in the mRNA. Such mutations are also referred to as ‘out of frame gene fusions’ and may result from various DNA rearrangements including but not limited to inversions, deletions, or translocations. As is understood by a skilled person, the coding strand (i.e., sense strand) of a gene is the strand comprising the sequence corresponding to the mRNA sequence. Out of frame gene fusions may encode the entire protein corresponding to the first gene or only a part thereof. The out of frame fusion with the coding strand of the second gene may result in a Frame (i.e., neoORF) (see, e.g., FIGS. 5 and 7 as exemplary embodiments). Given that for most genes the introns are much larger than the exons, in some embodiments the class II) mutation results from the fusion of two genes with a genomic junction that maps for each gene within an intron. If splicing were to proceed using the splice sites of the parental genes, the splice product may fuse the downstream partner within the frame of the upstream partner, which can lead to a neoORF. In preferred embodiments, the mutations result in a nucleic acid sequence encoding an mRNA comprising a start codon encoded by the first gene and a poly-(A) signal encoded by the second gene.

In some embodiments, class II) mutations are intragenic genomic rearrangements which result in a neoORF. For example, such mutations may lead to the fusion of exons of the same gene having different reading frames (see, e.g., FIG. 6 as an exemplary embodiment). Intragenic genomic rearrangements are known to a skilled person and include, but are not limited to, intragenic deletions, intragenic tandem duplications, intragenic dispersed duplications, intragenic inverted duplications, intragenic insertions, and intragenic inversions.

In some embodiments, the said intragenic genomic rearrangements lead to a rearrangement of the natural exon-intron structure of a known gene in the human genome. In some embodiments, the intragenic genomic rearrangements are exon duplications, wherein an exon or a part of an exon is duplicated. Preferably, the genomic rearrangement is an intragenic deletion or and intragenic tandem duplication. In a particularly preferred embodiment, the genomic rearrangement is an intragenic deletion.

Class III)

A second type of structural variant refers to DNA rearrangements resulting in new junctions of DNA sequences, wherein the rearrangement results in the fusion at least part of the coding strand (most often an intronic sequence, but exonic or other sequence is also possible) of a first gene to a second sequence selected from intergenic non-coding DNA or to the noncoding strand of a second gene. The fusion results in the coding strand of the first gene being 5′ of the second sequence. Such variants are also referred to herein as “class III” mutations. Unlike class II) mutations which fuse two genetic sequences having the same orientation (i.e., the coding strands from two genes are fused), class III) mutations refer to the fusion of a first gene with a second sequence that does not encode for a gene or does not encode for a gene in the same orientation as the first gene. We refer to these neoantigens as “Hidden Frame Neoantigens” since they cannot be accurately predicted based solely on the genomic DNA sequence because the transcription termination and splicing after fusion of two DNA segments is inherently unpredictable. In fact, we demonstrate that these hidden frames occur frequently, even in tumors previously characterized by a limited number of mutations. For example, less than 4% of glioblastoma patients were previously characterized as having a high mutational load (see, Hodges et al. Neuro Oncol. 2017 August; 19(8): 1047-1057).

This second sequence may be (intergenic) non-coding DNA. (Intergenic) non-coding DNA includes DNA which is not predicted to encode a protein. Such non-coding DNA includes repetitive DNA, as well as DNA that regulates expression (e.g., promoters, enhancer elements, etc) and DNA that encodes non-coding RNA (ncRNA). ncRNA refers to RNA that is not translated into protein and includes tRNA, rRNA, microRNAs, etc. See, e.g., FIG. 8 as an exemplary embodiment. The second sequence may be the noncoding strand of a second gene.

In preferred embodiments, the mutations result in a nucleic acid sequence encoding an mRNA comprising a start codon encoded by the first gene and a poly-(A) signal encoded by the second sequence. The poly-(A) signal encoded by the second sequence may also be referred to as a ‘cryptic’ polyadenylation signal since the poly-(A) signal (without the class III) mutation) is not normally associated with mRNA or a protein encoding sequence.

As is known to a skilled person, messenger RNA is polyadenylated with the addition of a 3′ poly-(A) tail. The poly-(A) tail is involved in a number of processes including nuclear export and protein translation. Polyadenylation signals near the 3′ end of mRNA direct the cell machinery to add a poly-(A) tail. The most common polyadenylation signal on the RNA is AAUAAA. However other variants also exist.

The sequences of such signals and methods for identifying such signals in nucleic acid sequences are well-known in the art and can be predicted by a number of different in silico methods. For example, the genomic sequence of the non-coding second sequence may be analyzed by a sequencing method, such as Illumina sequencing, or the like. In a second step the entire sequence assembled from individual sequencing reads may be screened in silico for the presence of known polyadenylation motifs/signal, e.g. using pattern matching, such as regular expressions, known by persons skilled in the art. Alternatively, one can experimentally test the presence of a poly-(A) tail at the 3′ end of an mRNA, by selecting the mRNAs by binding them to polyT oligonucleotides and removing all non-bound RNA. Using such selected mRNAs for high-throughput sequencing, preferably long-read sequencing, for example Nanopore sequencing, one can determine the sequences of all polyadenylated mRNAs in a tumor specimen or tumor cell. In preferred embodiments, the methods comprise selecting poly(A)-RNA. Such methods do not require a priori any knowledge of whether the corresponding encoding nucleic acid sequence comprises a poly(A) signal.

As is known to a skilled person, messenger RNA normally comprises a five-prime cap (5′ cap). In eukaryotes, mRNA is “capped” at the 5′ end with 7-methylguanylate during transcription. Methods for selecting and enriching for 5′ capped RNA are known in the art. For example, the TeloPrime Full-Length cDNA Amplification Kit V2 from Lexogen uses Cap-Dependent Linker Ligation (CDLL) and long reverse transcription (long RT) technology to select full-length RNA molecules that are both capped and polyadenylated. Other methods include the use of a mRNA 5′ Cap Structure Affinity Column Preparation as described in U.S. Pat. No. 6,187,544B1.

Class III) mutations represent a significant source of neoORFs (FIG. 9). Approximately one-third of the genome is made up of genes. This includes both strands of the DNA in both reading directions. Therefore, excluding biases and assuming randomness of breakpoints, one could estimate that the chance that a DNA rearrangement in or near a gene results in the fusion of two genes in the same orientation (such as class II) mutations) is around ⅙. The chance that the rearrangement fuses a gene to another sequence which is not a gene in the same orientation is around ⅚. We have calculated the amount of possible class I, II and III rearrangements (FIG. 9, FIG. 10) among 329 tumor cell lines. This revealed that for MSI-L tumors, the Frames are divided among the three classes as: Class I (20.5%), class II (20%), Class III (59.5%). The total number of rearrangements for all 270 MSI-L cell lines is 62,485, leading to 3553 Class II (5,7%) and 10,491 Class III (16,7%) Frames.

Preferably, the methods identify mutations from class I). Preferably, the methods identify mutations from class II). Preferably, the methods identify mutations from class III). Preferably, the methods identify mutations from classes I) and II). Preferably, the methods identify mutations from class I) and III). Preferably, the methods identify mutations from class II) and III), or rather the methods identify structural genomic variants. Preferably, the methods identify mutations from class I), II), and III). A skilled person will recognize that all classes of mutations may not be present in a particular tumor or that not all classes of mutations will be represented in the RNA of a tumor sample (see, e.g., Example 5). However, the methods are suitable for identifying such mutations.

As described above, Hidden Frames (class III) cannot be predicted based solely on the DNA sequence. The disclosure provides methods of identifying this new class of neoantigens. In a preferred embodiment, the method combines whole genome sequences with whole full-length transcriptome sequencing (in order to obtain the full-length sequence of intact mRNA). Preferably, the method uses three datasets:

1) whole genome sequencing to identify somatic structural variants from a tumor

2) full-length mRNA sequencing (usually between 20-100 million reads) from the tumor, preferably mRNAs having a 5′cap and poly-A tail and

3) (short) cDNA sequencing reads from the tumor.

In some embodiments, the candidate neoantigen sequences described herein may be identified by a method comprising performing long-read RNA sequencing on RNA or long-read sequencing on the corresponding cDNA from at least one tumor sample, preferably wherein RNA is poly-(A) selected mRNA and/or 5′ cap containing mRNA.

In some embodiments, the candidate neoantigen sequences described herein may be identified by a method, comprising

a) performing whole genome sequencing of a tumor sample and a healthy sample from the individual,
optionally performing long-read whole genome sequencing of a tumor sample and a healthy sample from the individual,

b) performing long-read RNA sequencing on RNA or long-read sequencing on the corresponding cDNA from at least one tumor sample, preferably wherein RNA is poly-(A) selected mRNA and/or 5′ cap containing mRNA;

c) identifying structural genomic variations in the tumor sample, using the whole genome sequencing data from (a);

d) determining the sequences of full-length RNA transcripts encoded by nucleic acid sequences comprising (or overlapping with) the somatic structural genomic variations;

e) determining the (predicted) amino acid sequences encoded by the full-length transcripts. Neoantigens useful for treatment comprise at least 9 contiguous amino acids of the (predicted) amino acid sequences, wherein at least four of the contiguous amino acids are not encoded in the germline genome of the individual.

In some embodiments, the methods described herein comprise performing whole genome sequencing of a tumor sample. In some embodiments, the method further comprises performing whole genome sequencing of a healthy sample (i.e., a non-tumorous sample) from the individual. Whole genome sequencing is generally performed using a short-read sequencing library (e.g., shotgun sequencing with paired-end sequencing reads of 2×150 bp). In preferred embodiments, the method comprises performing long-read whole genome sequencing on the tumor sample, either alone or preferably in combination with short-read whole genome sequencing. Long-read sequencing is especially useful for tumors having complex genomic rearrangements. Long-read sequencing may also be used to sequence a healthy sample. As described further herein, long-read sequencing methods are often referred to as “third generation sequencing” and include systems from Pacific Biosciences and Oxford Nanopore technologies. As a skilled person will recognize, when using highly accurate long-read sequencing techniques, short-read sequencing is redundant.

The methods identify somatic genomic changes that result in new open reading frames. The new open reading frames are not present in the germline genome of the individual. In some embodiments, the methods comprise comparing the nucleic acid sequences from at least one tumor sample with reference sequences. Sequence comparison can be performed by any suitable means available to the skilled person. Indeed, the skilled person is well equipped with methods to perform such comparison, for example using software tools like BLAST and the like, or specific software to align short or long sequence reads.

In some embodiments, the reference sequences are obtained from sequencing healthy tissue from said individual. A comparison of the sequences between a tumor sample and healthy tissue will identify somatic genomic mutations present in the tumor sample. This comparison often makes use of a comparison of the tumor and the healthy tissue sample to a reference human genome sequence (GRCh37, GRCh38, or the like). The differences with respect to the reference human genome sequence are subsequently compared between tumor and healthy tissue. This provides a list of genetic changes that solely occur in the tumor genome, often referred to as somatic genetic changes. In some embodiments, the reference sequence is a human reference genome such as GRCh37 (the Genome Reference Consortium human genome (build 37) date of release February 2009) or GRCh38 the Genome Reference Consortium human genome (build 38) date of release December 2013.

Analysis of sequence reads and identification of mutations will occur through standard methods in the field. For sequence alignment, aligners specific for short or long reads can be used, e.g. BWA (Li and Durbin, Bioinformatics. 2009 Jul. 15; 25(14):1754-60) or Minimap2 (Li, Bioinformatics. 2018 Sep. 15; 34(18):3094-3100). Subsequently, mutations can be derived from the read alignments and their comparison to a reference sequence using variant calling tools, for example Genome Analysis ToolKit (GATK), MuTect, Varscan, and the like (McKenna et al. Genome Res. 2010 September; 20(9):1297-303), which are often used for identification of short insertions and deletions (indels) or single nucleotide variations. Specific software is available for using read alignments for identification of large structural genomic rearrangements, including but not limited to deletions, duplications, inversions, insertions and translocations. An example of such software is GRIDSS, which uses split-read and read-pair mappings and retrieves the sequences of genomic rearrangement breakpoint-junctions through assembly of discordantly mapping sequence reads (Cameron et al. Genome Res 2017 27:2050-2060). Other existing software tools are Delly (Rausch et al. Bioinformatics 2012 28:i333-i339), or Manta (Chen et al. Bioinformatics 2016 32:1220-2), which are based on similar principles. An overview of the methods to identify genomic rearrangements in cancer genomes can be found in the paper by Kosugi et al (Kosugi et al. Genome Biol 2019 20:117). Following the identification of breakpoint-junctions of genomic rearrangements, one can perform an annotation step to identify Frames, i.e. determining the effects of the genomic rearrangement on the protein sequences, using known information on gene structure, transcript sequences, as available in e.g. the Ensembl database (http://www.ensembl.org/index.html). Methods for annotation of indels and genomic rearrangements resulting in class I and class II Frames are (for example) Annovar (Wang et al. Nucleic Acids Res 2010 38:e164) or Integrate-Neo (Zhang et al. Bioinformatics 2017 33:555-557).

A preferred method for identification of neoantigens, in particular class II and class III Frames, comprises the in silico reconstruction of rearranged genomic regions and resulting mRNA sequences by using whole genome sequencing, or more preferably a combination of whole genome sequencing and RNA sequencing. In some embodiments the method uses a combination of whole genome sequencing and ribosome profiling and RNA sequencing, or a combination of whole genome sequencing, long-read whole genome sequencing and ribosome profiling and short-read RNA sequencing and long-read RNA sequencing. An approach for analysis of the neoantigens, in particular class II/III Frames, based on such sequencing data, then may involve the following steps, or variations of these steps:

(i) mapping of genome sequencing data of tumor and healthy tissue to a reference human genome sequence, (ii) identification of genomic rearrangement breakpoint junctions from discordantly mapped sequence reads, (iii) assembling full length transcripts from RNA sequence reads that are spanning or in close vicinity to rearrangement breakpoint-junctions, (iv) identification of translation start sites in the assembled transcript sequences, (v) translation of neoORFs present in said assembled transcript sequences to predict associated protein sequences, and (vi) checking that said protein sequences are not present in any known human protein databases, by BLAST searches, or the like.

The methods further comprise determining the (predicted) amino acid sequences encoded by the new open reading frames. As is clear to a skilled person, this step may be performed when identifying somatic genomic changes.

In order to identify candidate neoantigen peptide sequences with the potential to induce an immune response, neoORFs comprising at least 9 contiguous amino acids are selected. A candidate neoantigen peptide sequence preferably comprises at least 9 contiguous amino acids encoded by a neoORF. Preferably, the candidate neoantigen peptide sequences comprise at least 15 or at least 20 or at least 25 or more contiguous amino acids encoded by a neoORF. In some embodiments, shorter neoantigen sequences comprising at least 4 amino acids encoded by a neoORF may also be useful. In those cases, candidate neoantigen peptide sequences comprise additional sequences flanking the neoORF encoded amino acids such that the candidate neoantigen peptide sequences comprise at least 9 amino acids (for binding to MHC class I), or up to 25 or more amino acids (for binding to MHC class II). FIG. 14 depicts two exemplary embodiments of i) a Frame of 20 amino acids and (ii) a shorter Frame of 4 amino acids, in combination with at least 5 amino acids of upstream in-frame sequence. While not wishing to be bound by theory, 9 amino acids is considered to be the minimum length of an MHC epitope and peptides having this length are likely to be more amenable to cellular processing and antigen presentation.

In preferred embodiments, the methods further comprise determining whether said neoORFs are expressed in a tumor sample. Expression of neoORFs can be determined by, e.g., determining the presence of the amino acids or peptides encoded by the neoORFs. Methods for determining the sequence of peptides, e.g., using mass spectrometry, are known to a skilled person.

Expression can also be determined by sequencing RNA from at least one tumor sample from the individual. In some embodiments, the sequence of the RNA overlapping the new junctions of DNA sequences resulting from said DNA rearrangements and/or the sequence of the RNA overlapping the frameshift mutation is determined. In some embodiments, the entire RNA molecule comprising a neoORF is sequenced.

General methods for mRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using a purification kit, buffer set and protease from commercial manufacturers, such as Qiagen, according to the manufacturer's instructions (QIAGEN Inc., Valencia, Calif.). For example, total RNA from cells in culture can be isolated using Qiagen RNeasy mini-columns. Numerous RNA isolation kits are commercially available and can be used in the methods of the invention.

Preferably, the RNA isolated for sequencing is cytosolic RNA that is not tRNA or rRNA. Preferably, the RNA is poly-(A)RNA. Methods for selecting poly-(A) RNA are known to a skilled person and include mixing total RNA with poly-(T) oligomers and retaining only the RNA that is bound to the poly-(T) oligomers. Preferably, the RNA is selected for having a 5′-CAP. More preferably, the RNA is selected for having a 5′-CAP and a 3′-poly-(A) tail (FIG. 25).

In some embodiments, the RNA is reversed transcribed to cDNA and the cDNA is sequenced. In some embodiments direct RNA sequencing is performed. “RNA sequencing” and “RNA sequences” as used herein encompass both direct RNA sequencing and cDNA sequences from the corresponding RNA.

In some embodiments, short-read sequencing methods such as sequencing-by-ligation (SBL) and sequencing-by-synthesis (SBS) are used. Generally, such short-read sequencing methods provide read lengths of around 100-200 bases. These methods are also referred to as second-generation sequencing or Next-generation sequencing.

While second-generation (or short-read) sequencing provides highly accurate sequence information, in some cases it can be difficult to correctly annotate longer stretches of sequences, in particular when such sequences involve repetitive elements or complex rearrangements. Long-read sequencing has the advantage that longer stretches of nucleic acid can be sequenced. Preferably, long-read sequencing methods are used to determine RNA sequence as well as DNA sequence. Such methods are often referred to as “third generation sequencing” and include systems from Pacific Biosciences and Oxford Nanopore technologies.

While short-read sequencing is useful to confirm the RNA expression of at least a part of a neoORF, long read sequencing offers the advantage that the structure of the entire mRNA molecule can usually be determined. An example of the diversity of mRNA molecules present for a gene, is shown in FIG. 19. Determining the full-length structure of mRNA molecules containing indel mutations and genomic rearrangements is essential to identify Frame neopeptide sequences. This is especially useful for class II and III mutations. In regards to class II (gene fusions), the splicing pattern of a gene depends on the structure of the primary transcript. Preferably, long read sequencing is used to confirm the splicing events of the gene fusion. In regards to class III mutations, long read sequencing is preferably also used to confirm that a polyadenylated RNA is produced, and to determine possible (cryptic) splicing patterns. An example of cryptic splicing for a class III Frame (Hidden Frame) is shown in FIG. 24, FIG. 28 and FIG. 30.

Preferably, the long-read molecules that are sequenced are at least 300 nucleotides in length, more preferably at least 500 nucleotides in length, more preferably covering the full-length mRNA molecules for each expressed gene in a tumor sample. To obtain molecules for long read sequencing the RNA is generally not fragmented during isolation and purification. Methods for sequencing long-read RNA molecules are well-known in the art and are disclosed in publications such as Tilgner, H. et al., Proc. Nat'l Acad. Sci., USA, 111(27):9869-9874 (2014), Tseng, E. and Underwood, J., J. Biomol. Techniques., 24 Supplement: 545 (2013), Sharon, D., et al., Nature Biotech. 31(10):1009-1014 (2013), Pan. Q., et al., Nature Genetics, 40:1413-1415 (2008), Steijger, T., et al., Nature Methods, 10:1177-1184 (2013) and U.S. Pat. Nos. 8,192,961, 8,501,405 and 8,940,507, all of which are incorporated by reference. Similar methods are useful for long-read whole genome sequencing (see also Logsdon, Nature Reviews Genetics 2020). Preferably, long-read single molecule DNA and/or RNA sequencing technologies are used in the present methods. Such methods can generate reads of at least 1 kb even tens to thousands of kilobases in length. The accuracy of such methods is constantly improving and, as a skilled person will appreciate, if highly accurate long-read sequence data is available, then short-read sequencing is redundant.

For example, long-read sequencing on a Pacific Biosciences sequencer enables Circular Consensus Sequencing (CCS), which involves repeated sequencing of the same template DNA molecule (or cDNA molecule). The repeated sequences can be collapsed to generate a highly accurate consensus sequence, which reaches a sequence accuracy competitive with short-read (RNA) sequencing methods. Circular consensus sequencing involves the generation of long sequence reads with (inverted) tandemly repeated copies of the original transcript molecule. Such concatemer reads can be used to generate a high-quality consensus sequences. Examples of such approach are described in e.g. Wenger et al, Nature Biotechnology volume 37, pages 1155-1162(2019). Generation of high-quality mRNA transcript reads with such approach have been described (see review by Byrne et al, Philos Trans R Soc Lond B Biol Sci. 2019 Nov. 25; 374(1786): 20190097). Once highly accurate consensus sequences are obtained, each individual consensus read (which corresponds to a single mRNA molecule) can be directly translated.

An alternative approach involves the polishing of long cDNA reads with short highly accurate cDNA sequence reads, termed hybrid correction (Lima et al, https://doi.org/10.1101/476622). Such methods typically correct each individual long transcript read with overlapping short cDNA reads, providing accurate hybrid-corrected long transcript reads, which can be directly translated into a protein sequence.

In preferred embodiments, the method comprises selecting as candidate neoantigen peptide sequences, peptide sequences whose corresponding RNA, preferably poly-(A) and 5′-capped RNA, sequence is present in the tumor sample.

The identification of neoantigens resulting from genomic rearrangements can be difficult if the identification method only makes used of DNA sequencing, since the junction at the DNA level is most often not included in the mature mRNA, and the junction in the mRNA between the ‘old’ gene and the flanking sequence is not to be found in the DNA because it was created by splicing. In many cases it is not possible to predict the neoantigen based solely on the DNA sequence.

As explained further herein, Hidden Frames cannot be predicted based solely on DNA sequence using standard methods. The resulting Frame will depend not only on the DNA rearrangement (i.e., structural variation) but also on the splicing machinery. The vast majority of DNA rearrangements occur in non-coding DNA, e.g., in the non-coding region of a gene (e.g., an intron). The sequences immediately surrounding the rearrangement junction will therefore normally not correspond to the splicing junction in the resulting mRNA and will normally not be present in the resulting corresponding mRNA (see, e.g., FIG. 8). Methods provided herein comprise determining the sequences of full-length RNA transcripts encoded by nucleic acid sequences comprising (or overlapping with) the somatic DNA rearrangements. As is clear to a skilled person, sequences immediately surrounding the DNA rearrangement junction will normally not be represented in the full-length RNA transcripts.

Accordingly, the methods disclosed herein are particularly useful for identifying neoantigen sequences that result from DNA rearrangements. In a preferred embodiment the method identifies structural genomic variations such as:
DNA rearrangements resulting in new junctions of DNA sequences, wherein the rearrangement results in the fusion of at least part of the coding strand of a first gene to at least part of the coding strand of a second gene or the rearrangement results in an intragenic genomic rearrangement, wherein said DNA rearrangement results in a change of the reading frame of a polypeptide encoding sequence, and
DNA rearrangements resulting in new junctions of DNA sequences, wherein the rearrangement results in the fusion of at least part of the coding strand of a first gene to intergenic non-coding DNA or to the noncoding strand of a second gene, preferably wherein said intergenic non-coding DNA or noncoding strand comprises a poly-(A) signal.

In one embodiment, a method, referred to herein as ‘FramePro’ or ‘reconstructed tumor genome mapping’, comprises the generation of a tumor-specific human reference genome, based on somatic and germline structural genome variations identified in a tumor sample, followed by mapping of long cDNA/RNA reads to the tumor-specific reference sequences. The method comprises the following steps:

a) Whole genome sequencing (WGS) of a tumor sample and a healthy sample from the individual as described further herein. Preferably, WGS of the tumor sample includes long-read sequencing. As demonstrated in example 13, long-read genome sequencing allows reconstruction of complex DNA rearrangements.

b) Long-read RNA sequencing of RNA from at least one tumor sample as described further herein. Preferably the RNA is selected or enriched for poly-(A) mRNA and/or 5′-CAP containing mRNA as described further herein (see also FIG. 25).

c) Optionally performing short-read RNA sequencing on RNA from at least one tumor sample as described further herein.

d) Mapping the genomic sequences obtained to a human reference sequence to identify somatic structural genomic variations in the tumor sample as described further herein. In a preferred embodiment the genomic sequences are mapped to a reference human genome sequence (GRCh37, GRCh38, or the like). This step also distinguishes germline genetic variations (identified from the healthy tissues) from tumor-specific genetic variations (identified from the tumor tissue) as discussed herein.

e) Generating in silico a reconstructed tumor-specific reference genome comprising the identified somatic structural genomic variations. As will be understood by the skilled person, it is not necessary to generate a complete tumor-specific reference genome. Rather, contigs which span the structural genomic variations can be generated (see, e.g., FIG. 26). Such contigs are generally around 100 kb but can be longer, e.g., 300-400 kb. Longer contigs may be useful in genomic regions which comprise a large number or re-arrangements. The reconstructed tumor-specific reference genome contigs can be generated by any method known to a skilled person. For example, the genomic DNA segments from the reference human genome sequence can be joined based on the information on breakpoint junctions derived from the WGS (e.g., using SV variant calling) Alternatively, the WGS data comprising the SVs may be directly used in an assembly algorithm to generate assembled contigs covering the rearranged segments.

f) Aligning the RNA sequences to the reconstructed tumor-specific reference genome. As described in Example 12, this step is useful when mapping RNA sequencing data to the genome. The cancer tumor often comprises complex rearrangements which complicate that mapping of RNA sequences, in particular as the order and orientation of exonic sequences in the tumor genome may be different than in the human reference genome. As shown in FIG. 40, mapping short-read RNA sequencing data to the human GRCh37 reference failed to identify transcript reads derived from an intragenic tandem duplication in the KLF5 gene. However, the novel RNA junctions and transcript structure is found when mapping long-read RNA sequencing reads to a reconstructed tumor-specific contig.

In some embodiments, this step is an iterative process comprising short-read sequencing data and long-read sequencing data to the reconstructed contigs. The short-read data can be used to polish (i.e., correct) the long-read data. The long-read data is particularly useful to determine the correct splicing pattern of the transcripts, which cannot be reliably predicted by only analysing the predicted intron-exon junctions at a DNA level. In turn, the short-read data precisely determine each separate splice-junction, enabling polishing of the long RNA sequencing reads and the splice-junction patterns identified therein. Long read data also allows the identification of multiple, alternative transcripts (see, e.g., “Isoform identification” from FIG. 27 and FIG. 30.).

g) Determining the sequences of the full-length RNA transcripts encoded by the structural genomic variations. The present disclosure provides that when the transcription/splicing machinery encounters a DNA rearrangement, it will often seek new splice sites resulting in an RNA transcript with a novel open reading frame. Based on the WGS and RNA sequencing data provided above, the sequence of these new RNA transcripts can be determined.

In some embodiments, the step involves determining the sequence of the full-length RNA transcripts directly from the RNA sequencing data. This may be accomplished, e.g., when highly accurate long-read sequence data is available. In some embodiments, this step involves determining the sequence of the full-length RNA transcripts based on the reconstructed tumor-specific reference genome using the information regarding splice junctions obtained from the RNA sequencing data. As discussed further herein, multiple full-length RNA transcripts may be encoded by genomic sequences comprising SVs (see, e.g., “Isoform identification” from FIG. 27).

h) Determining the predicted amino acid sequences encoded by the full-length transcripts of g) as further described herein. This method provides an improved pipeline for determining tumor neoantigens, in particular for neoantigens resulting from complex chromosomal rearrangements. This method can also be used to select for such tumor neoantigens (referred to herein as Frames) by:

i) Selecting, as candidate neoantigen sequences, sequences comprising at least 9 contiguous amino acids of the predicted amino acid sequence of h), wherein at least four of the contiguous amino acids are not encoded in the germline genome of the individual, as further described herein.

In one embodiment, a method, which we refer to herein as ‘direct-RNA Frame detection’ is provided. Said method comprises the mapping of cDNA/RNA sequencing reads to a normal human reference genome, such as GRCh37, GRCh38 or the like, followed by identification of a possible ‘path’ following genomic rearrangement breakpoint-junctions in the tumor genome that could lead to a contig that places the mapped cDNA/RNA segments together in a small genomic sequence (arbitrarily defined as smaller than e.g. 200 kb) (FIG. 44). Such method is particularly relevant for identification of Frames emerging from complex genomic rearrangements, such as chromothripsis or the like, which occurs at high-frequency in many human cancers (Cortes-ciriano et al, Nature Genetics volume 52, pages 331-341(2020). Complexity of genomic rearrangements may not be fully resolved by short-read WGS or long-read WGS, which makes mapping of long cDNA/RNA reads to the normal human reference a relevant alternative option. The method may involve the following steps or combinations of steps:

a. Long-read RNA or cDNA sequencing of RNA from a tumor sample as described further herein. Preferably the RNA is selected or enriched for poly(A) mRNA and/or 5′ cap containing mRNA as described further herein.

b. Optionally performing short-read RNA or cDNA sequencing on RNA from at least one tumor sample as described further herein.

c. Aligning the RNA/cDNA sequences to the reference genome, such as GRCh37, GRCh38 or alternative human reference genomes. In some embodiments, the short-read RNA data can be used to polish (i.e., correct) the long-read RNA data before alignment to the reference genome.

d. Whole genome sequencing (WGS) of a tumor sample and a healthy sample from the individual as described further herein. Preferably, WGS of the tumor sample includes long-read sequencing, as long-read sequencing may improve the identification and resolving of complex DNA rearrangements (Cretu Stancu et al, Nature Communications 8, 1326 (2017); Nattestad et al, Genome Research 2018 August; 28(8):1126-1135).

e. Mapping the genomic sequences obtained from WGS to a human reference sequence to identify somatic structural genomic variations in the tumor sample as described further herein. In a preferred embodiment the genomic sequences are mapped to a reference human genome sequence (GRCh37, GRCh38, or the like). This step also distinguishes germline genetic variations (identified from the healthy tissues) from tumor-specific genetic variations (identified from the tumor tissue) as discussed herein.

f. In some embodiments, the method comprises identification of a possible linear contig of DNA sequence in the tumor genome sequences that comprises the genomic segments to which the long cDNA/RNA transcript sequence reads are aligned. The order and orientation of said genomic segments should be in agreement with the order and orientation of the exons that are observed in the long transcript read(s) (FIG. 44). The contig may be between 10 kb-1,000 kb, preferably at least 50 kb and on average between 100-300 kb.

g. Generating in silico a reconstructed tumor-specific reference genome comprising the identified genomic segments to which the long-read RNA/cDNA exons align. As will be understood by the skilled person, it is not necessary to generate a complete tumor-specific reference genome. Rather, contigs which span the mapped long-read RNA segments can be generated (FIG. 26, FIG. 44). Such contigs are generally around 100 kb but can be longer, e.g., 300-400 kb. Longer contigs may be useful if the corresponding transcripts span long distances, e.g. because of large intron sizes. The reconstructed tumor-specific reference genome contigs can be generated by any method known to a skilled person. Preferably, the genomic DNA segments (to which RNA segments align) from the reference human genome sequence can be joined based on the information on breakpoint junctions derived from the WGS (e.g., using structural variant calling). Alternatively, tumor-specific reference contigs can be generated by joining the genomic DNA segments (along with some flanking sequence) to which long-read RNA/cDNA exons align.

h. Aligning the RNA sequences to the reconstructed tumor-specific contigs. In some embodiments, this is a multi-step process comprising mapping short-read RNA/cDNA sequencing data and long-read RNA/cDNA sequencing data to the reconstructed contigs. The short-read RNA data can be used to polish (i.e., correct) the long-read RNA data before the mapping of the long-read RNA/cDNA data and/or after the mapping of the long-read RNA/cDNA data.

i. Determining the sequences of the full-length RNA transcripts encoded by the structural genomic variations. The present disclosure provides that when the transcription/splicing machinery encounters a DNA rearrangement, it will often seek new splice sites resulting in an RNA transcript with a novel open reading frame. Based on the WGS and RNA sequencing data provided above, the sequence of these new RNA transcripts can be determined. In some embodiments, the step involves determining the sequence of the full-length RNA transcripts directly from the (polished) RNA sequencing data. This may be accomplished, e.g., when highly accurate long-read sequence data is available. In some embodiments, this step involves determining the sequence of the full-length RNA transcripts based on the reconstructed tumor-specific reference genome using the information regarding splice junctions obtained from the RNA sequencing data.

j. Determining the predicted amino acid sequences encoded by the full-length transcripts of i) as further described herein.

This method provides an improved pipeline for determining tumor neoantigens, in particular for neoantigens resulting from complex chromosomal rearrangements. This method can also be used to select for such tumor neoantigens by:

k. Selecting, as candidate neoantigen sequences, sequences comprising at least 9 contiguous amino acids of the predicted amino acid sequence of j), wherein at least four of the contiguous amino acids are not encoded in the germline genome of the individual, as further described herein.

As will be apparent to one of skill in the art, the methods described herein are preferably performed with the aid of a computer. In particular, as is clear to a skilled person, the mapping and/or aligning of such extensive sequencing reads requires the use of computer programs, which are known in the art.

As described further herein, the methods described above are particularly useful for identifying the “Framome” of a tumor, which can then be used in the preparation of a vaccine, or other form of immunotherapy, including but not limited to cellular immunotherapy.

The disclosure further provides methods for preparing a vaccine, collection of vaccines, or collection of neoantigens for the immunotherapy-based treatment of cancer in an individual, comprising identifying candidate neoantigen peptide sequences as disclosed herein. Vaccine or collections are prepared comprising peptides having the candidate neoantigen amino acid sequences or comprising nucleic acids encoding said amino acid sequences. Preferably, the vaccine or collection comprises at least 3, at least 4, at least 5, at least 10, at least 15, or at least 20, or at least 50 neoantigens/Frames.

The disclosure provides vaccines, collections of vaccines, and collection of neoantigens for the treatment of cancer obtainable by identifying candidate neoantigens as disclosed herein. The vaccines and collections may comprise peptides having said candidate neoantigen peptide sequences or nucleic acids encoding said peptide sequences. As described herein, said candidate neoantigen peptide sequences may include the entire, or essentially the entire, Framome, or a selection may be made as described herein.

Preferably, vaccines and collections disclosed herein induce an immune response, or rather the neoantigens are immunogenic. Preferably, the neoantigens bind to an antibody or a T-cell receptor. In preferred embodiments, the neoantigens comprise an MHCI or MHCII ligand/epitope.

The major histocompatibility complex (MHC) is a set of cell surface molecules encoded by a large gene family in vertebrates. In humans, MHC is also referred to as human leukocyte antigen (HLA). An MHC molecule displays an antigen and presents it to the immune system of the vertebrate. Antigens (also referred to herein as ‘MHC ligands’) bind MHC molecules via a binding motif specific for the MHC molecule. Such binding motifs have been characterized and can be identified in proteins. See for a review Meydan et al. 2013 BMC Bioinformatics 14:S13.

MHC-class I molecules typically present the antigen to CD8 positive T-cells whereas MHC-class II molecules present the antigen to CD4 positive T-cells. The terms “cellular immune response” and “cellular response” or similar terms refer to an immune response directed to cells characterized by presentation of an antigen with class I or class II MHC involving T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

In preferred embodiments, the present disclosure involves the stimulation of an anti-tumor CTL response against tumor cells expressing one or more tumor-expressed antigens (i.e., Frames) and preferably presenting such tumor-expressed antigens with class I MHC.

Frames may be analysed by known means in the art in order to identify potential MHC binding peptides (i.e., MHC ligands). Suitable methods are described herein in the examples and include in silico prediction methods (e.g., ANNPRED, BIMAS, EPIMHC, HLABIND, IEDB, KISS, MULTIPRED, NetMHC, PEPVAC, POPI, PREDEP, RANKPEP, SVMHC, SVRMHC, and SYFFPEITHI, see Lundegaard 2010 130:309-318 for a review). MHC binding predictions depend on HLA genotypes, furthermore it is well known in the art that different MHC binding prediction programs predict different MHC affinities for a given epitope.

As will be clear to a skilled person, the neoantigen sequences may also be provided as a collection of tiled sequences, wherein such a collection comprises two or more peptides that have an overlapping sequence. Such ‘tiled’ peptides have the advantage that several peptides can be easily synthetically produced, while still covering a large portion of the Frame. In an exemplary embodiment, a collection comprising at least 3, 4, 5, 6, 10, or more tiled peptides each having between 10-50, preferably 12-45, more preferably 15-35 amino acids, is provided. As will be clear to a skilled person, a collection of tiled peptides comprising a candidate neoantigen peptide sequence indicates that when aligning the tiled peptides and removing the overlapping sequences, the resulting tiled peptides provide the amino acid sequence of the candidate sequence, albeit present on separate peptides.

In some embodiments, the entire candidate neoantigen peptide sequence (i.e., Frame) may be provided as the vaccine (e.g., peptide or nucleic acid). Preferred Frames are at least 9 amino acids in length, more preferably at least 20 amino acids in length, more preferably at least 30 amino acids, and most preferably at least 50 amino acids in length. While not wishing to be bound by theory, it is believed that neoantigens longer than 10 amino acids can be processed into shorter peptides, e.g., by antigen presenting cells, which then bind to MHC molecules.

In some embodiments, fragments of a Frame can also be presented as the neoantigen. The fragments comprise at least 8 consecutive amino acids of the Frame, preferably at least 10 consecutive amino acids, and more preferably at least 20 consecutive amino acids, and most preferably at least 30 amino acids. In some embodiments, the fragments can be about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, or about 120 amino acids or greater. Preferably, the fragment is between 8-50, between 8-30, or between 10-20 amino acids. As will be understood by the skilled person, fragments greater than about 10 amino acids can be processed to shorter peptides, e.g., by antigen presenting cells.

In some embodiments, the neoantigens (i.e., peptides) are directly linked. Preferably, the neoantigens are linked by peptide bonds, or rather, the neoantigens are present in a single polypeptide. Accordingly, the disclosure provides polypeptides comprising at least two peptides (i.e., neoantigens). In some embodiments, the polypeptide comprises 3, 4, 5, 6, 7, 8, 9, 10 or more peptides (i.e., neoantigens). In an exemplary embodiment, a polypeptide may comprise 10 different neoantigens, each neoantigen having between 10-400 amino acids. Thus, the polypeptide may comprise between 100-4000 amino acids, or more. As is clear to a skilled person, the final length of the polypeptide is determined by the number of neoantigens selected and their respective lengths. A collection may comprise two or more polypeptides comprising the neoantigens which can be used to reduce the size of each of the polypeptides.

In some embodiments, the amino acid sequences of the neoantigens are located directly adjacent to each other in the polypeptide. For example, a nucleic acid molecule may be provided that encodes multiple neoantigens in the same reading frame. In some embodiments, a linker amino acid sequence may be present. Preferably a linker has a length of 1, 2, 3, 4 or 5, or more amino acids. The use of linker may be beneficial, for example for introducing, among others, signal peptides or cleavage sites. In some embodiments at least one, preferably all of the linker amino acid sequences have the amino acid sequence VDD.

As will be appreciated by the skilled person, the peptides and polypeptides disclosed herein may contain additional amino acids, for example at the N- or C-terminus. Such additional amino acids include, e.g., purification or affinity tags or hydrophilic amino acids in order to decrease the hydrophobicity of the peptide. In some embodiments, the neoantigens may comprise amino acids corresponding to the adjacent, wild-type amino acid sequences of the relevant gene, e.g., amino acid sequences located 5′ to the frame shift mutation that results in the neo open reading frame. Preferably, each neoantigen comprises no more than 20, more preferably no more than 10, and most preferably no more than 5 of such wild-type amino acid sequences.

The peptides and polypeptides can be produced by any method known to a skilled person. In some embodiments, the peptides and polypeptide are chemically synthesized. The peptides and polypeptide can also be produced using molecular genetic techniques, such as by inserting a nucleic acid into an expression vector, introducing the expression vector into a host cell, and expressing the peptide. Preferably, such peptides and polypeptide are isolated, or rather, substantially isolated from other polypeptides, cellular components, or impurities. The peptide and polypeptide can be isolated from other (poly)peptides as a result of solid phase protein synthesis, for example. Alternatively, the peptides and polypeptide can be substantially isolated from other proteins after cell lysis from recombinant production (e.g., using HPLC).

The disclosure further provides nucleic acid molecules encoding the peptides and polypeptide disclosed herein. Based on the genetic code, a skilled person can determine the nucleic acid sequences which encode the (poly)peptides disclosed herein. Based on the degeneracy of the genetic code, sixty-four codons may be used to encode twenty amino acids and translation termination signal.

In a preferred embodiment, the nucleic acid molecules are codon optimized. As is known to a skilled person, codon usage bias in different organisms can affect gene expression level. Various computational tools are available to the skilled person in order to optimize codon usage depending on which organism the desired nucleic acid will be expressed. Preferably, the nucleic acid molecules are optimized for expression in mammalian cells, preferably in human cells. Table 2 lists for each acid amino acid (and the stop codon) the most frequently used codon as encountered in the human exome.

TABLE 2
most frequently used codon for
each amino acid and most
frequently used stop codon.

| | |
|---|---|
| A | GCC |
| C | TGC |
| D | GAC |
| E | GAG |
| F | TTC |
| G | GGC |
| H | CAC |
| I | ATC |
| K | AAG |
| L | CTG |
| M | ATG |
| N | AAC |
| P | CCC |
| Q | CAG |
| R | CGG |
| S | AGC |
| T | ACC |
| V | GTG |
| W | TGG |
| Y | TAC |
| Stop | TGA |

In preferred embodiments, at least 50%, 60%, 70%, 80%, 90%, or 100% of the amino acids are encoded by a codon corresponding to a codon presented in Table 2.

The disclosure further provides vectors comprising the nucleic acids molecules disclosed herein. A “vector” is a recombinant nucleic acid construct, such as plasmid, phase genome, virus genome, cosmid, or artificial chromosome, to which another nucleic acid segment may be attached. The term “vector” includes both viral and non-viral means for introducing the nucleic acid into a cell in vitro, ex vivo or in vivo. The disclosure contemplates both DNA and RNA vectors. The disclosure further includes self-replicating RNA with (virus-derived) replicons, including but not limited to mRNA molecules derived from mRNA molecules from alphavirus genomes, such as the Sindbis, Semliki Forest and Venezuelan equine encephalitis viruses.

Vectors, including plasmid vectors, eukaryotic viral vectors and expression vectors are known to the skilled person. Vectors may be used to express a recombinant gene construct in eukaryotic cells depending on the preference and judgment of the skilled practitioner (see, for example, Sambrook et al., Chapter 16). For example, many viral vectors are known in the art including, for example, retroviruses, adeno-associated viruses, and adenoviruses. Other viruses useful for introduction of a gene into a cell include, but are not limited to, adenovirus, arenavirus, herpes virus, mumps virus, poliovirus, Sindbis virus, and vaccinia virus, such as, canary pox virus. The methods for producing replication-deficient viral particles and for manipulating the viral genomes are well known. In preferred embodiments, the vaccine comprises an attenuated or inactivated viral vector comprising a nucleic acid disclosed herein.

Preferred vectors are expression vectors. It is within the purview of a skilled person to prepare suitable expression vectors for expressing the inhibitors disclosed hereon. An “expression vector” is generally a DNA element, often of circular structure, having the ability to replicate autonomously in a desired host cell, or to integrate into a host cell genome and also possessing certain well-known features which, for example, permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art. Suitable regulatory sequences including enhancers, promoters, translation initiation signals, and polyadenylation signals may be included. Additionally, depending on the host cell chosen and the vector employed, other sequences, such as an origin of replication, additional DNA restriction sites, enhancers, and sequences conferring inducibility of transcription may be incorporated into the expression vector. The expression vectors may also contain a selectable marker gene which facilitates the selection of host cells transformed or transfected. Examples of selectable marker genes are genes encoding a protein such as G418 and hygromycin which confer resistance to certain drugs, β-galactosidase, chloramphenicol acetyltransferase, and firefly luciferase.

The expression vector can also be an RNA element that contains the sequences required to initiate translation in the desired reading frame, and possibly additional elements that are known to stabilize or contribute to replicate the RNA molecules after administration. Therefore, when used herein, the terms DNA and RNA when referring to an isolated nucleic acid encoding a neoantigen peptide should be interpreted as referring to DNA from which the peptide can be transcribed or RNA molecules from which the peptide can be translated.

Also provided for is a host cell comprising a nucleic acid molecule or a vector as disclosed herein. The nucleic acid molecule may be introduced into a cell (prokaryotic or eukaryotic) by standard methods. As used herein, the terms “transformation” and “transfection” are intended to refer to a variety of art recognized techniques to introduce a DNA into a host cell. Such methods include, for example, transfection, including, but not limited to, liposome-polybrene, DEAE dextran-mediated transfection, electroporation, calcium phosphate precipitation, microinjection, or velocity driven microprojectiles (“biolistics”). Such techniques are well known by one skilled in the art. See, Sambrook et al. (1989) Molecular Cloning: A Laboratory Manaual (2 ed. Cold Spring Harbor Lab Press, Plainview, N.Y.). Alternatively, one could use a system that delivers the DNA construct in a gene delivery vehicle. The gene delivery vehicle may be viral or chemical. Various viral gene delivery vehicles can be used with the present invention. In general, viral vectors are composed of viral particles derived from naturally occurring viruses. The naturally occurring virus has been genetically modified to be replication defective and does not generate additional infectious viruses, or it may be a virus that is known to be attenuated and does not have unacceptable side effects.

Preferably, the host cell is a mammalian cell, such as MRC5 cells (human cell line derived from lung tissue), HuH7 cells (human liver cell line), CHO-cells (Chinese Hamster Ovary), COS-cells (derived from monkey kidney (African green monkey), Vero-cells (kidney epithelial cells extracted from African green monkey), Hela-cells (human cell line), BHK-cells (baby hamster kidney cells, HEK-cells (Human Embryonic Kidney), NSO-cells (Murine myeloma cell line), C127-cells (nontumorigenic mouse cell line), PerC6®-cells (human cell line, Crucell), and Madin-Darby Canine Kidney (MDCK) cells. In some embodiments, the disclosure comprises an in vitro cell culture of mammalian cells expressing the neoantigens obtained as disclosed herein. Such cultures are useful, for example, in the production of cell-based vaccines, such as viral vectors expressing the neoantigens disclosed herein.

As is clear to a skilled person, if multiple neoantigens are used, they may be provided in a single vaccine composition or in several different vaccines to make up a vaccine collection. The disclosure thus provides vaccine collections comprising a collection of tiled peptides, collection of peptides, as well as nucleic acid molecules, vectors, or host cells. As is clear to a skilled person, such vaccine collections may be administered to an individual simultaneously or consecutively (e.g., on the same day) or they may be administered several days or weeks apart.

Various known methods may be used to administer the vaccines to an individual in need thereof. For instance, one or more neoantigens can be provided as a nucleic acid molecule directly, as “naked DNA”. Neoantigens can also be expressed by attenuated viral hosts, such as vaccinia or fowlpox. This approach involves the use of a virus as a vector to express nucleotide sequences that encode the neoantigen. Upon introduction into the individual, the recombinant virus expresses the neoantigen peptide, and thereby elicits a host CTL response. Vaccination using viral vectors is well-known to a skilled person and vaccinia vectors and methods useful in immunization protocols are described in, e.g., U.S. Pat. No. 4,722,848. Another vector is BCG (Bacille Calmette Guerin) as described in Stover et al. (Nature 351:456-460 (1991)).

Preferably, the vaccine comprises a pharmaceutically acceptable excipient and/or an adjuvant. The compositions may contain pharmaceutically acceptable auxiliary substances as required to approximate physiological conditions, such as pH adjusting and buffering agents, tonicity adjusting agents, wetting agents and the like. Suitable adjuvants are well-known in the art and include, aluminum (or a salt thereof, e.g., aluminium phosphate and aluminium hydroxide), monophosphoryl lipid A, squalene (e.g., MF59), and cytosine phosphoguanine (CpG). A skilled person is able to determine the appropriate adjuvant, if necessary, and an immune-effective amount thereof. As used herein, an immune-effective amount of adjuvant refers to the amount needed to increase the vaccine's immunogenicity in order to achieve the desired effect

Preferably, the vaccine or collection of vaccines comprises all of the candidate neoantigen peptide sequences identified in the tumor sample of an individual. Preferably, the vaccine or collection of vaccines comprises all of the candidate neoantigen peptide sequences identified in the tumor sample of an individual which are also expressed in the tumor (e.g., RNA encoding said neoantigens is present in the tumor). While not wishing to be bound by theory, the use of the full Framome as a vaccine is believed to increase the success rate of the vaccine.

The vaccines disclosed herein are preferably designed to maximize the number of neoantigen amino acids provided (either as peptides or nucleic acids encoding said peptides) to an individual afflicted with cancer. In some embodiments, the vaccine is an F100 product, i.e., the vaccine comprises at least 100 neoantigen amino acids encoded in the tumor genome and resulting from neoORFs (Framome), preferably, detected in the RNA of the tumor. In some embodiments, the vaccine is an F200, F500, or F1000 product, i.e., the vaccine comprises at least 200, 500, or 1000, respectively, neoantigen amino acids encoded in the tumor genome and, preferably, detected in the RNA of the tumor. See, e.g., FIG. 22.

In some embodiments, there may be reasons to select a subset of the Framome for preparation of a vaccine. For example, if the vaccine is produced as a peptide, or collection of peptides, then a set of between 5-20 peptides preferably having between 20-30 amino acids per peptide may be used. In which case, such an exemplary vaccine would cover a Framome of between 100-500 amino acids.

In some embodiments, the neoantigens are selected based on cysteine content. As known to a skilled person, when the vaccine is a synthetic peptide, or collection of synthetic peptides, the amino acid content may be evaluated to determine whether peptide synthesis and mixing of peptides is possible. Peptide cysteine content is an important factor since cysteines can form disulfide bridges, which may lower solubility and trigger clutting. Frames with the lowest cysteine content are therefore preferred. The simplest method for determining cystein content is defined as Qcys=N/L, where N is defined as the number of Cysteines in a Frame and L the total length in amino acids of the Frame. However, other methods are considered as well, for example the number of subsequences of a Frame of defined length L, which have a cysteine content (Q) larger than a predefined value, where Lϵ{5, 6, 7, 8, 9, 10, 11, . . . , n} with n being the entire length of the Frame sequence in amino acids, and Q being the cysteine content of a Frame subsequence defined as above (N/L). In preferred embodiments, the cysteine content for each peptide is 30% or less, more preferably, 5% or less.

In some embodiments, “self-peptides” are not included in the neoantigen vaccine or collection. Preferably, the candidate neoantigen peptide sequences do not share a contiguous stretch of at least 6 amino acids with human protein reference sequences. Such human reference sequences are available at the NCBI RefSeq database. Other protein databases for identifying a matching pattern include, for example uniprot (https://www.uniprot.org/) or proteomics databases (https://www.proteomicsdb.org/).

In some embodiments, candidate neoantigen sequences are selected on the basis of genomic variant allele frequency (VAF), to select clonal (or truncal) neoantigen sequences, i.e. neoantigens present in all tumor cells of a tumor and not in only a subset of the tumor cells. As used herein, VAF is defined as: VAF=Rmut/Rtot where Rmut is the number of sequencing reads in the genome sequencing data containing the frameshift mutation or genomic rearrangement breakpoint junctions, and Rtot is the total number of sequencing reads covering the frameshift mutation locus. A corrected VAF (VAFcor) can be subsequently calculated based on the estimated tumor purity. Preferably, candidate sequences have a VAF or VAFcor of at least 0.1, more preferably >0.1, more preferably >0.2.

In some embodiments, candidate neoantigen sequences are selected which are predicted to comprise an MHC I or MHC II binding epitope, as disclosed further herein.

In some embodiments, candidate neoantigen sequences are selected to optimize the physical spread of Frames across the chromosomes. In particular, candidate neoantigen sequences are selected for which the underlying somatic mutations have a maximum distance with regard to chromosomal location. While not wishing to be bound by theory, a single neoORF may be lost, for example via chromosome loss or deletion. However, the chance that two neoORFs located on different chromosomal arms are both lost is highly unlikely. The use of neoORFs distally located from each other is therefore a useful strategy to reduce the risk of antigen loss. The selection of such neoORFs may be useful if the use of the full Framome as a vaccine has practical limitations.

There are multiple ways to choose a set of Frames based on their chromosomal locations. One possible approach is as follows. Let d be the number of Frames to be selected. Let F={f₁, f₂, . . . , fₙ} be the set of all Frames within a patient. Let c_{f}_{ᵢ} correspond to the chromosome of frame fᵢ. Let

$$V = ( \begin{matrix}F \\ d\end{matrix} ) = \{ {v}_{1} , {v}_{2} , … , {v}_{k} \}$$

be the set of unique subsets of d Frames taken from F. The preferred combination of Frames is

$${v}_{preffered} = {arg max}_{v ∈ V} {\sum }_{a ∈ v} {\sum }_{b ∈ v} ( 1 - {δ}_{{c}_{a} , {c}_{b}} )$$

In some embodiments, neoantigen peptide sequences are selected wherein each somatic mutation corresponding to the neoantigen is located on a different chromosomal arm.

In preferred embodiments, the vaccine or collection comprises all of the candidate neoantigen peptide sequences identified in the tumor sample of an individual which are also expressed in the tumor (e.g., RNA encoding said neoantigens is present in the tumor) and which are not “self-peptides” as disclosed herein.

In preferred embodiments, the vaccine or collection comprises all of the candidate neoantigen peptide sequences identified in the tumor sample of an individual which are also expressed in the tumor (e.g., RNA encoding said neoantigens is present in the tumor), which are not “self-peptides” as disclosed herein, and have a VAF or VAFcor of at least 0.1.

In preferred embodiments, the vaccine or collection comprises all of the candidate neoantigen peptide sequences identified in the tumor sample of an individual which are also expressed in the tumor (e.g., RNA encoding said neoantigens is present in the tumor) and have a VAF or VAFcor of at least 0.1.

In preferred embodiments, the vaccine or collection comprises all of the candidate neoantigen peptide sequences identified in the tumor sample of an individual which are also expressed in the tumor (e.g., RNA encoding said neoantigens is present in the tumor), which are not “self-peptides” as disclosed herein, have a VAF or VAFcor of at least 0.1, and comprise a predicted MHC I or MHC II binding epitope.

The disclosure also provides the use of the neoantigens disclosed herein for the treatment of disease, in particular for the treatment of cancer in an individual. It is within the purview of a skilled person to diagnose an individual with as having cancer. In a preferred embodiment, the cancer is not Microsatellite instable (MSI), in particular the cancer is not MSI-H (i.e., high amount of microsatellite instability). MSI is due to defects in DNA mismatch repair. MSI screening tests are available which analyse changes in the DNA sequence between normal tissue and tumor tissue and can identify the level of instability. In some embodiments, MSI-H cancer is defined as the presence of mutations in 30% or more of microsatellites.

As used herein, the terms “treatment,” “treat,” and “treating” refer to reversing, alleviating, or inhibiting the progress of a disease, or reversing, alleviating, delaying the onset of, or inhibiting one or more symptoms thereof. Treatment includes, e.g., slowing the growth of a tumor, reducing the size of a tumor, and/or slowing or preventing tumor metastasis.

As used herein, administration or administering in the context of treatment or therapy of a subject is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners.

The optimum amount of each neoantigen to be included in the vaccine composition and the optimum dosing regimen can be determined by one skilled in the art without undue experimentation. The composition may be prepared for injection of the peptide, nucleic acid molecule encoding the peptide, or any other carrier comprising such (such as a virus or liposomes). For example, doses of between 1 and 500 mg 50 μg and 1.5 mg, preferably 125 μg to 500 μg, of peptide or DNA may be given and will depend from the respective peptide or DNA. Other methods of administration are known to the skilled person. Preferably, the vaccines may be administered parenterally, e.g., intravenously, subcutaneously, intradermally, intramuscularly, or otherwise.

For therapeutic use, administration may begin at or shortly after the surgical removal of tumors. This can be followed by boosting doses until at least symptoms are substantially abated and for a period thereafter.

In some embodiments, the vaccines may be provided as a neoadjuvant therapy, e.g., prior to the removal of tumors or prior to treatment with radiation or chemotherapy. Neoadjuvant therapy is intended to reduce the size of the tumor before more radical treatment is used.

The vaccines are preferably capable of initiating a specific T-cell response. It is within the purview of a skilled person to measure such T-cell responses either in vivo or in vitro, e.g. by analyzing IFN-γ production or tumor killing by T-cells. In therapeutic applications, vaccines are administered to a patient in an amount sufficient to elicit an effective CTL response to the tumor antigen and to cure or at least partially arrest symptoms and/or complications.

The vaccines can be administered alone or in combination with other therapeutic agents. The therapeutic agent is for example, a chemotherapeutic agent, radiation, or immunotherapy, including but not limited to checkpoint inhibitors, such as nivolumab, ipilimumab, pembrolizumab, or the like. Any suitable therapeutic treatment for a particular, cancer may be administered.

The term “chemotherapeutic agent” refers to a compound that inhibits or prevents the viability and/or function of cells, and/or causes destruction of cells (cell death), and/or exerts anti-tumor/anti-proliferative effects. The term also includes agents that cause a cytostatic effect only and not a mere cytotoxic effect. Examples of chemotherapeutic agents include, but are not limited to bleomycin, capecitabine, carboplatin, cisplatin, cyclophosphamide, docetaxel, doxorubicin, etoposide, interferon alpha, irinotecan, lansoprazole, levamisole, methotrexate, metoclopramide, mitomycin, omeprazole, ondansetron, paclitaxel, pilocarpine, rituxitnab, tamoxifen, taxol, trastuzumab, vinblastine, and vinorelbine tartrate.

Preferably, the other therapeutic agent is an anti-immunosuppressive/immunostimulatory agent, such as anti-CTLA antibody or anti-PD-1 or anti-PD-L1. Blockade of CTLA-4 or PD-L1 by antibodies can enhance the immune response to cancerous cells. In particular, CTLA-4 blockade has been shown effective when following a vaccination protocol.

As is understood by a skilled person the vaccine and other therapeutic agents may be provided simultaneously, separately, or sequentially. In some embodiments, the vaccine may be provided several days or several weeks prior to or following treatment with one or more other therapeutic agents. The combination therapy may result in an additive or synergistic therapeutic effect.

The compounds and compositions disclosed herein are useful as therapy and in therapeutic treatments and may thus be useful as medicaments and used in a method of preparing a medicament.

In some embodiments, the disclosure provides methods for the preparation of a cellular immunotherapy, such as personalized neoantigen-specific T-cell therapy. Such cellular immunotherapy is directed against the tumor cells with expressed Frames where Frame-derived peptides are presented in complexes with HLA molecules on the cell surface.

Various methods for the use of neoantigen-specific T-cells or neoantigen-specific T-cell receptors in cancer immunotherapy have been described. T-cell receptors (TCRs) are expressed on the surface of T-cells and consist of an α chain and a β chain. TCRs recognize antigens bound to MHC molecules expressed on the surface of antigen-presenting cells. The T-cell receptor (TCR) is a heterodimeric protein, in the majority of cases (95%) consisting of a variable alpha (a) and beta (6) chain, and is expressed on the plasma membrane of T-cells. The TCR is subdivided in three domains: an extracellular domain, a transmembrane domain and a short intracellular domain. The extracellular domain of both a and 6 chains have an immunoglobulin-like structure, containing a variable and a constant region. The variable region recognizes processed peptides, among which neoantigens, presented by major histocompatibility complex (MHC) molecules, and is highly variable. The intracellular domain of the TCR is very short, and needs to interact with CD3ζ to allow for signal propagation upon ligation of the extracellular domain.

The major histocompatibility complex (MHC) is a set of cell surface molecules encoded by a large gene family in vertebrates. In humans, MHC is also referred to as human leukocyte antigen (HLA). An MHC molecule displays an antigen and presents it to the immune system of the vertebrate. Antigens (also referred to herein as ‘MHC ligands’) bind MHC molecules via a binding motif specific for the MHC molecule. Such binding motifs have been characterized and can be identified in proteins. See for a review Meydan et al. 2013 BMC Bioinformatics 14:S13.

MHC-class I molecules typically present the antigen to CD8 positive T-cells whereas MHC-class II molecules present the antigen to CD4 positive T-cells. The terms “cellular immune response” and “cellular response” or similar terms refer to an immune response directed to cells characterized by presentation of an antigen with class I or class II MHC involving T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

With the focus of cancer treatment shifted towards more targeted therapies, among which immunotherapy, the potential of therapeutic application of tumor-directed T-cells is increasingly explored. Such strategies involve the analysis of T-cell receptors (TCRs), either based on T-cells obtained from a tumor specimen, or based on peripheral T-cells from a cancer patient. In vitro characterization of TCRs present on T cells found in tumor specimens or peripheral blood, for their specificity against specific Frame neoantigens could be used to select specific TCR sequences that can be used for development of immunotherapy. Such TCR sequences can, for example, be used for development of TCR-like antibodies (Støkken Høydahl et al, Antibodies 2019, 8, 32). Identified and isolated TCR sequences can also be used for engineering of T-cells, so as to provide them with a specific TCR that recognizes a neoantigen. Several methods for T-cell engineering have been described in the art, including methods to improve the function of T-cells with regard to safety, tumor infiltration and immune stimulation (Rath et al, Cells 2020, 9, 1485).

The disclosure provides methods comprising contacting T-cells with HLA molecules, preferably MHC-I, bound to one or more of the candidate neoantigen peptide sequences identified from an individual according to the methods described herein. In particular, such methods for identifying neoantigens combine whole genome sequencing with long-read RNA/cDNA sequencing to identify neoantigen sequences. The neoantigen peptides used as “bait” are preferably selected based on the potential to bind MHC. Suitable methods to predict MHC binding include in silico prediction methods (e.g., ANNPRED, BIMAS, EPIMHC, HLABIND, IEDB, KISS, MULTIPRED, NetMHC, PEPVAC, POPI, PREDEP, RANKPEP, SVMHC, SVRMHC, and SYFFPEITHI, see Lundegaard 2010 130:309-318 for a review).

In some embodiments, a method is provided that comprises the (i) isolation of T-cells from a tumor specimen (e.g. tumor-infiltrating lymphocytes), peripheral blood, bone marrow, lymph node tissue, or spleen tissue from an individual afflicted with cancer, (ii) identification of Frame neoantigens using methods as described herein, (iii) prediction of MHC class I binding epitopes within the Frame neoantigens sequences, (iv) preparation of Frame peptide— MHC (pMHC) multimers, (v) selection of T-cells using the pMHC molecules. Preferably, the method further comprises the (vi) expansion of selected T-cells using appropriate culture conditions. More preferable the method comprises the infusion of the selected or expanded T-cells back into the patient.

Methods for the selection and identification of immune cells, preferably T-cells or T-cell receptors with specificity for neoantigens are well-known in the art (see e.g. reviews by Bianchi et al, Front Immunol. 2020; 11: 1215 and Zhao and Cao, Frontiers in Immunology, 2019, https://doi.org/10.3389/fimmu.2019.02250, as well as US20180000913, which is hereby incorporated by reference). For example, predicted MHC-I binding epitopes from the Frame neoantigens are bound to synthetic tetrameric forms of fluorescently labelled MHC Class I molecules. CD8+ T-cells with the appropriate T cell receptor will bind to the labelled tetramers and can be selected by flow cytometry. Other suitable methods include those described in U.S. Pat. No. 7,125,964. Briefly, recombinantly produced biotinylated MHC molecules are attached to avidin coated magnetic beads. Peptides and T-cells are added to the beads. T-cells absorbed to the beads (via the interaction with a peptide-MHC complex) are selected.

In some embodiments, the disclosure provides methods which are not a treatment of the human or animal body and/or methods that do not comprise a process for modifying the germ line genetic identity of a human being.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The invention is further explained in the following examples. These examples do not limit the scope of the invention, but merely serve to clarify the invention.

### EXAMPLES

### Example 1

We screened over 10,000 sequenced tumors, of all major cancer types, to determine the fraction of tumors that have at least one frameshift-caused encoded neopeptide of at least 10 amino acids (abbreviated as one “Frame”), and also the fraction that has at least two Frames, three Frames, etc. Even when excluding microsatellite instable (MSI) tumors (which contain many frameshift indels), we find that the major fraction of tumors contains one or more Frames. For example, in lung cancer 95% of patients have at least one Frame derived from frameshift indels and 75% have a least four Frames.

As shown in FIG. 2, a significant number of newly encoded amino acids are caused by frameshift mutations arising from short insertions and deletions (indels). We refer to such mutations herein as ‘class I’ mutations and FIG. 3 depicts an exemplary frame-shift mutation and the resulting neo-open reading frame (i.e., Frame sequence). It is reasonable that where frameshift mutations cause two thirds of the newly encoded amino acids, on average they also cause roughly two thirds of the antigenicity of the tumor.

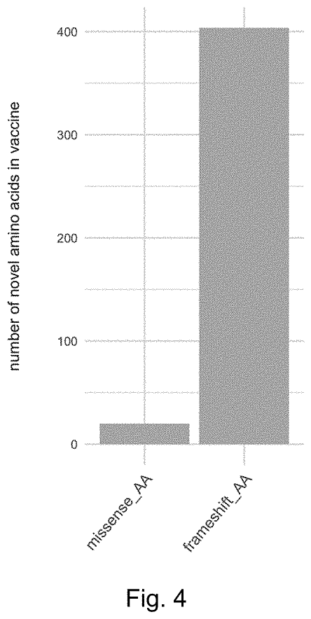

On top of that, as explained above vaccination against SNV-neoantigens is forced to focus on only a fraction of the antigenicity that resides in the sum of all SNV-encoded antigens. This is depicted in FIG. 4.

A second source of Frames is represented by out-of-frame fusions, resulting from inter-genic genomic rearrangements. We refer to these mutations as ‘class II’ (see FIG. 5, FIG. 7).

Other mutations that belong to class II result from intra-genic deletions which result in the fusion of exons that have different reading frames (FIG. 6, FIG. 7). Both intra-genic fusions and inter-genic fusions are collectively referred to as class II mutations.

A third class of Frame neoantigens results from the induction of expression, by a 5′ region of a gene, of a region of genomic DNA that is not known to contain a gene. Such may occur, for example, if a DNA rearrangement fuses a 5′ part of a gene to a random (non-genic) piece of genomic DNA. This is referred to as ‘class III’ mutations or Hidden Frames (FIG. 8).

It is expected that expression of polyadenylated mRNA encoding Frames may rely on the presence of a (cryptic) poly-adenylation signal in the random (non-genic) piece of genomic DNA. The presence of a poly-(A) tail in the transcribed sequence for a class III frame, increases the chance that such transcript is translatable into a (novel) protein, consisting of part of a known gene and a translated genomic sequence not known as a gene. Class III frames have not been systematically described elsewhere, but represent an important reservoir of neoantigens. This third class of Frames may increase the number of Frames with a further 60%, beyond indel frame-shifts (class I) and out-of-frame fusions (class II) for MSI-L tumors (FIG. 9A). The number of potential novel amino acids comprised by class III Frames further advances the total Framome size of a tumor, on average, with 50% for MSI-L tumors (FIG. 9B).

We have explored the CCLE cancer cell lines collection to understand the relative contribution of Frame neoantigens caused by class I, II and III Frames. As can be observed in FIG. 10A and FIG. 10B, the relative contribution of Frame classes, I, II, and III differs substantially per tumor cell line. For MSI-H tumors (right side of the plot), class I Frames are the dominant class, while for the non-MSI-H tumors (left part of the plot), class III is the dominant class.

The number of Frames per tumor genome determines the practical use of the Framome as anti-cancer vaccine. It turns out that a large proportion of tumors contain Frames. FIG. 12 shows the analysis of the numbers of indel Frames (class I) per tumor genome, for all cancers in the TCGA database. For example, as can be seen, 95% of all lung cancers (LUSC) contain 1 or more indel Frames, 80% 3 or more, 50% contains 6 or more indel Frames etc. These numbers can become two- to ten-fold higher, when also including class II and class III mutations as a source of Frames, depending on tumor type and highly varying per individual tumor (FIG. 10).

The size of the (average) Framome of tumors of various types has not been described. The average length of Frames is a priori approximately 20 amino acids⁶, since DNA has 64 triplets of which 3 are stops, approximately 1 in 20. We determined the average length of the Framome for class I Frames: see FIG. 13. Notice that since we defined Frames for operational reasons as encoded neo-peptides of at least ten amino acids in length, the smaller neo-peptides are not counted, and as a result the average length of the Framome is slightly smaller than the simple product of 20 times the average number of frameshifts within ORFs. As shown in FIG. 13 the average length of the indel-based Framome in lung cancer is 257 (LUAD) or 259 (LUAD) amino acids, the average length in bladder cancer is 182 (BLCA), in kidney 160 (KIRC) and 202 (KIRP).

For cancer vaccination therapy based on missense mutations, every novel amino acid requires a unique sequence in the form of a synthetic peptide or DNA/RNA encoding such peptide. For the Framome, however, it is within reach to use the entire Framome for vaccination purposes. FIGS. 16-18 show the entire Framome (class I, II, III), for several tumor cell lines based on the CCLE data collection. This shows that with only a few peptide sequences (or DNA/RNA sequences encoding those peptide sequences), the entire Framome (or a large part of the Framome) of a tumor can be covered.

### Example 2

We performed an analysis of the Framome for a series of human cancer cell lines from the Cancer Cell Line Encyclopedia (CCLE, https://portals.broadinstitute.org/ccle). Frames were predicted for known frameshift indel mutations in these cell lines and annotated based on the criteria described above. Subsequently Frames were selected based on hard cut-offs for each of the criteria:
peptide length >=10
no match to any self protein with length >7 amino acids
genomic Variant Allele Frequency >0.2
expression observed by at least 1 RNAseq read
a cysteine content <0.1
match to at least one weak or strong binding MHC epitope

Note that each of these criteria can be varied, based on setting different cutoff values and by using different methods to determine each of the respective parameters.

By applying the above settings to a series of cancer cell lines, we retrieved a selected set of class I Frames that could be used as a personalized cancer vaccine. The total number of class I Frames within a cell line and the numbers following each filtering step are shown in FIG. 15.

### Example 3

The following steps describe an exemplary design of a Framome vaccine based on a cancer patient's mutation report.

1. Extract all somatic class I, II, and III mutations from the mutation report.

2. Determine the expression of the class I, II, and III mutations identified in the mutation report, by means of RNA sequencing

3. Determine the entire transcript structures for messenger RNAs that contain frameshift mutations, e.g. using long-read RNA sequencing of poly-(A) selected mRNAs.

4. Project them onto the reference human genome sequence to derive the resulting new open reading frame peptides Koster, J. & Plasterk, R. H. A. A library of Neo Open Reading Frame peptides (NOPs) as a sustainable resource of common neoantigens in up to 50% of cancer patients. Sci. Rep. 9, 6577 (2019).

5. Remove those that cause a new open reading frame shorter than N amino acids, where N can be set at 4, 5, 6 or more amino acids.

6. Screen the resulting newly encoded peptides against the products of the human ORFeome and filter out those that have a match of more than M amino acids, to avoid self-antigens, where M can be set at 5, 6, 7 or more amino acids. The remaining peptide sequences are referred to as Frames.

7. Rank the Frames by the criteria mentioned above.

8. When the vaccine consists of synthetic long peptides, the top ranking sequences defined under point 5 will be considered with the sum of the length of the top ranking sequences being <Q amino acids, where Q can be set at a practical number, e.g. 300 amino acids. Frames longer than 30 amino acids will be covered by a tiling array of 30-mer synthetic peptides, so that no epitope is lost because it happens to be on the edge of a single peptide.

Some Frames may occur in genes that are more often hit by frameshift mutations as described in recent literature (Koster, J. & Plasterk, supra). We would prefer to include those Frames, because they can be provided off-the-shelf, and the same vaccine product can be applied to different patients that have frameshifts leading to the same Frame.

### Example 4

Defining the peptide sequences of Frames is dependent on analysis of translatable mRNA sequences encoding such Frame peptides. To obtain full length sequences of mRNAs one can use long-read single molecule sequencing methods, such as, but not limited to, Pacific Biosciences or Oxford Nanopore sequencing.

We have used Oxford Nanopore sequencing to identify Frame encoding mRNAs in a mouse tumor, derived from tumor cell line MC38, a colon carcinoma. In brief, total RNA was isolated from MC38 tumor tissue using Macherey Nagel NucleoSpin RNA extraction methods. Subsequently, poly-(A) mRNA was selected from the total RNA using poly-(T) dynabeads (Thermo Scientific). Poly-(A) mRNA was used as input for preparation of a cDNA library, according to the protocol (SQK-DCS109) for use with Oxford Nanopore sequencing. Said cDNA library was sequenced on a MinION Nanopore sequencer, resulting in approximately 8 million transcript sequences. Transcript sequences were mapped to the human reference genome (GRCh37) using Minimap2 to identify the genomic positions of individual transcripts. In addition, exome sequencing data were generated for mouse tumor cell line MC38 and corresponding healthy tissue from the same genetic background (C57/BL6) using known methods in the art, based on Illumina sequencing. Somatic indel mutations were identified in the MC38 tumor genome. One of such indels is present in the Pdxk gene (deletion of T base at position 10: 78441188 in mouse reference genome MM10). About one third of Pdxk transcripts is derived from the indel allele (FIG. 20) and some of these use alternative exons or a shorter 3′UTR sequence, which was used to predict the exact Frame peptide sequences resulting from this indel. Similar approaches can be used for defining Frame peptide sequences for class II and III Frames.

### Example 5

To test the use of the methodology described herein, we collected a tumor sample from a patient with pancreatic cancer. Genomic DNA was extracted from the tumor sample and the corresponding blood cells of the same patient, using established procedures (Macherey Nagel NuceoSpin or Qiagen DNeasy spin columns). DNA was used for whole genome paired-end sequencing (2×150 bp reads) on Illumina NovaSeq instruments to an average coverage depth of 100× for the tumor sample and 30× for the corresponding blood sample. In addition, total RNA was isolated from the tumor sample using Macherey Nagel NucleoSpin RNA extraction methods. Subsequently, poly-(A) mRNA was selected from the total RNA using poly-(T) dynabeads (Thermo Scientific). Poly-(A) mRNA was used as input for preparation of a cDNA library, according to the protocol (SQK-DCS109) for use with Oxford Nanopore sequencing. Approximately 2.5 million long RNA sequencing reads were generated, with an average length of ˜1000 bp. Total RNA was used for short-read RNA sequencing on Illumina NovaSeq, following ribosomal RNA depletion of total RNA and preparation of a short-read RNA sequencing library from the ribosomal RNA depleted RNA using Illumina TruSeq protocols. Approximately 50 million short paired-end RNA sequencing reads were generated. Whole genome sequencing data were analysed using existing bioinformatics methods to identify somatic genetic changes (e.g. as described by Priestley et al, Nature 575, pages 210-216, 2019). Based on this analysis, the tumor sample contains 132 predicted genomic rearrangements and 3 short intra-exonic indel mutations. Said rearrangements and indel mutations were annotated using Ensembl gene annotations, to identify Class I, II and III Frames, resulting in the identification of 0, 9, and 39 class I, II and III Frames, respectively (FIG. 23). The expression of the Frames was determined using the short and long-read RNA sequencing data, demonstrating the presence of expressed Frames. In addition, the long-read RNA sequencing data confirmed messenger RNA sequences and resulting translated Frame products thereof (FIG. 24). Altogether, this procedure identified class II and III Frames that could form the basis of a cancer vaccine for this patient with pancreatic cancer and shows the broad applicability of class II and class III Frames for cancer vaccination.

### Example 6

To further test the general use of the methodology described herein, we collected tumor samples from two patients with lung cancer. Genomic DNA and RNA was extracted and sequenced as described in example 5, reaching 100× coverage for the genome sequencing of the tumors and 30× coverage for the corresponding normal tissue samples from the same patients. For long read Nanopore RNA sequencing, we generated 3 Gb and 7.4 Gb of data for the Lung tumor 1 and Lung tumor 2, respectively. Following data analysis as previously described (e.g. as described by Priestley et al, Nature 575, pages 210-216, 2019), we determined all class I, II and III Frames. We found 0, 6, and 17 class I, II and III Frames for Lung tumor 1 and 0, 3 and 22 class I, II and III Frames for Lung tumor 2. The expression of Frames was established using the short and long-read RNA sequencing data, demonstrating the presence of expressed Frames. In addition, the long-read RNA sequencing data confirmed messenger RNA sequences and resulting translated Frame products thereof, similarly as shown for the pancreas tumor described in FIG. 23 and FIG. 24. This further confirms the broad applicability of class II and class III Frames for cancer vaccination.

### Example 7

In order to improve the prediction of neoantigens resulting from SVs, the FramePro pipeline was developed. In an exemplary embodiment of the FramePro pipeline, the following five datasets are utilized:

1. long read whole genome sequence of the tumor genome to determine the correct configuration of (complex) genomic rearrangements in the tumor genome

2. short read whole genome sequence of the tumor genome to ensure the precision of that sequence, since the error rate of long-read genome sequencing is relatively high

3. long read RNA (cDNA) sequencing of poly-(A) and 5′-CAP-containing (and thus full length and mature) mRNA extracted from tumor cells, to ensure the proper combination of splice variants within each transcript

4. short read RNA (cDNA) sequencing of mRNA extracted from tumor cells, to ensure the precise sequence of the mature mRNA, including the precise splice-junctions

5. Short-read or long-read whole genome sequencing of the genome of normal cells from the same patient for whom the tumor genome and transcriptome is sequenced

A preferred example for carrying out the FramePro method for identifying tumor neoantigens is as follows.

### Experimental Methodology Description

1. Isolation of genomic DNA and total RNA from tumor tissue or tumor cells. To obtain high quality genomic DNA and total RNA from a tumor sample, a joint DNA/RNA isolation procedure was developed involving:
   a. tissue grinding/homogenization
   b. splitting of the sample in a portion for DNA and RNA isolation
   c. total RNA isolation and long fragment DNA isolation
2. Whole genome sequencing of tumor genomic DNA and genomic DNA from corresponding control tissue (blood, saliva, normal tissue), involving:
   a. preparation of a short-read sequencing library
   b. high-throughput (short-read) paired-end sequencing of said sequencing library to a coverage depth of the genome of at least 1-fold
   c. high-throughput long-read sequencing in addition to or as an alternative to the short-read whole genome sequencing to a coverage depth of at least 1-fold
3. Long-read transcriptome sequencing of tumor RNA, involving:
   a. Selection of polyadenylated RNA molecules from the total RNA prep using oligo-dT probes.
   b. Selection of RNA molecules with a 5′Cap structure from the polyadenylated RNA molecules obtained from step 3, e.g. using existing technology on the market (e.g. TeloPrime).
   c. Conversion of selected RNA molecules with poly-(A) 3′-end and 5′-Cap into double stranded cDNA.
   d. Optional amplification of double-stranded cDNA from step 3c by a limited number of PCR cycles.
   e. Preparation of a sequencing library from the double-stranded cDNA obtained from step 3c or step 3d. The sequencing library can be prepared according to any available protocol for long-read sequencing, including but not limited to Oxford Nanopore sequencing, or Pacific Biosciences sequencing.
   f. Sequencing of the cDNA library on a long-read sequencing instrument, such as Oxford Nanopore MinION/GridION/PromethION or Pacific Biosciences RSII or Sequel.
4. Short-read transcriptome sequencing of tumor RNA, involving:
   a. Selection of mRNA molecules by:
      i. oligo-dT selection of the total RNA to enrich for polyadenylated mRNAs, and/or 5′Cap selection of total RNA
      ii. Depletion of abundant ribosomal RNA molecules by selective removal of ribosomal RNA, e.g. using complementary probes and RNAse H digestion
   b. Preparation of a short-read RNA sequencing library of selected mRNA molecules, by conversion of the mRNA to double stranded cDNA and adapter ligation, based on protocols known in the art.
   c. Sequencing of said short-read RNA sequencing library on a short-read sequencing instrument, such as Illumina HiSeq, NextSeq or the like.

Bioinformatic Analysis Description:
5. Calling of germline and somatic genetic variations based on the whole genome short-read and/or long-read sequencing data according to best practices in the field using known software tools such as Mutect, GATK, Varscan, GRIDSS, PURPLE, or the like, involving:
   a. calling of single nucleotide variants (SNVs)
   b. calling of short insertions and deletions (indels)
   c. calling of large structural variants (SVs)
   d. calling of and copy number variations (CNVs)
6. Generation of a locally reconstructed genome of the tumor, based on the variant calls obtained from step 5 and/or the long-read sequencing data, using FramePro software, involving:
   a. joining of genomic DNA segments from the reference genome into long contigs, informed by the breakpoint junctions derived from the SV variant calling, or
   b. short-read and/or long-read genome sequencing data containing breakpoint junctions may be directly used in an assembly algorithm (e.g. Miniasm, wtdbg2, or the like) to generate assembled contigs covering rearranged genomic segments.
7. Alignment of short-read RNA sequencing data to the reconstructed contigs obtained in step 6. A preferred aligner is STAR, but any short-read aligner that takes split mapping of exon-exon junctions into account will be of use.
8. Alignment of long-read cDNA sequencing data to the reconstructed contigs obtained in step 6, using long-read cDNA sequencing aligners such as Minimap2 or similar tools.
9. Identification of aligned short-read and long-read RNA reads across breakpoint junctions in the reconstructed genomic contigs from step 6.
10. Polishing of the long-read RNA sequencing data using highly accurate short-read RNA sequencing data, using tools such as TALC, or the like.
11. Correction of the splice-junctions of the polished long-read RNA reads using the highly accurate short-read RNA sequencing data, using e.g. FLAIR or similar tools.
12. Generation of full-length transcript sequences from polished and corrected single long cDNA/RNA sequencing reads, by concatenating sequences of the reference genome corresponding to each aligned exonic segment in said long cDNA/RNA read.
13. Addition of germline and/or somatic genetic variations (SNVs, short indels) in the corrected and polished long cDNA/RNA sequencing reads from step 12. This step involves genotyping each individual long cDNA/RNA sequencing read to determine whether genetic variations are present in the open read frame.
14. Identification of the upstream (5′) Translation Start Site (TSS), either based on annotation in known databases (Ensembl or the like) or by identification of possible open Reading Frames within the polished and corrected long cDNA/RNA read
15. Determining the novel portion of the protein sequence predicted from the polished and corrected cDNA/RNA read, which is referred to as a Frame peptide sequence (Frames).
16. Selection of all Frames from a tumor sample and the use of the entire collection of Frames (termed Framome) as a cancer immunotherapy, preferably cancer vaccines.

### Example 8 FramePro Analysis of a Mouse Tumor

The genome of a mouse tumor cell line (MC38) and the corresponding C57BL6 reference strain was sequenced using short-read (2*150 bp) whole genome sequencing to a coverage depth of 30× on Illumina HiSeq. The transcriptome of the MC38 tumor grown in mice was also sequenced following the preparation of a cDNA library using the Roche Kappa mRNA prep kit. The cDNA library was sequenced on Illumina HiSeq generating approximately 50M paired reads (2*150 bp). In addition, we prepared the total RNA for long-read sequencing based on selection of polyadenylated mRNA molecules using oligo-dT probes. Around 200 ng of polyadenylated mRNA was used for preparation of an Oxford Nanopore sequencing library using kit SQK-DCS109 and 13.5 Gb of data (11M reads) were generated on a Nanopore MinION sequencer.

Structural genomic variations (SVs) were called in the mouse whole genome sequencing data using Manta (https://github.com/Illumina/manta), run in tumor-normal mode. An extension of the Mouse reference genome (GRCm38) was generated by the formation of tumor-specific contigs based on the identified somatic SVs in the MC38 genome (FIG. 26). For each breakpoint-junction that includes a 5′-end of known gene, the DNA segment containing the 5′ end of the gene up to the breakpoint was joined to the flanking region on the other side of the breakpoint junction and this region was extended to a specific maximum value (often set at 500 Mb), or until a subsequent breakpoint-junction was encountered, upon which a further extension of the joined genomic segment was performed. The total contig size was typically set at 1.5 Mb, but can be adapted to any meaningful value. As a result of this procedure, for each gene containing a breakpoint, a new contig was formed, which was appended to the GRCm38 reference genome.

The short-read RNA sequencing data obtained from MC38 were aligned to the appended mouse reference genome using STAR (https://github.com/alexdobin/STAR), to obtain a list of all splice-junctions in the MC38 RNA sequencing data (FIG. 27). The erroneous long-read Oxford Nanopore MC38 RNA sequencing data were corrected using the short-read RNA-sequencing data using TALC (https://www.biorxiv.org/content/10.1101/2020.01.10.901728v2.full). Subsequently, the corrected mouse MC38 long-read RNA sequencing data were aligned to the same appended reference using Minimap2 (FIG. 27, FIG. 28). The alignment file (BAM) of the corrected MC38 long-read RNA sequencing data was used together with the MC38 short-read splice junctions to correct the long-read RNA splice junctions using FLAIR (https://github.com/BrooksLabUCSC/flair), since the long-read splice junctions may be off by one or a few bases because of the errors in the Nanopore sequencing. For each aligned and corrected Nanopore read bridging a breakpoint junction, the translation start site of the fused 5′-gene-end was taken to translate the aligned segments into a protein sequence. The C-terminal novel part of the protein sequence that is extending beyond the known fused 5′-gene-end is regarded as the Frame sequence (FIG. 28). A full Framome of the mouse MC38 cell line is depicted in FIG. 29.

Example 9. FramePro analysis of a lung tumor. We have sequenced the genome of a lung tumor using short-read (2*150 bp) whole genome sequencing to a coverage depth of 100× on Illumina HiSeq. Similarly, the corresponding germline genome was also sequenced to a coverage depth of 30×. The transcriptome of the lung tumor was sequenced using short-read RNA-sequencing, following the preparation of a cDNA library using the Roche Kappa mRNA prep kit. The cDNA library was sequenced on Illumina HiSeq generating approximately 182M paired reads (2*150 bp). In addition, we prepared the total RNA for long-read sequencing by first performing selection of polyadenylated mRNA molecules using oligo-dT probes and subsequent generation of Capped mRNAs using TeloPrime procedure, which generates double-stranded cDNA only for mRNA molecules with a 5′Cap structure. Around 200 ng of polyadenylated and capped mRNA was used for preparation of an Oxford Nanopore sequencing library using kit SQK-LSK109. Approximately 20 Gb of data (15M reads) were generated on a Nanopore MinION sequencer.

All classes of genetic variations were called in the short-read whole genome sequencing data using an existing read mapping to reference GRCh37 and variant calling pipeline: https://github.com/hartwigmedical/. An extension of the GRCh37 reference genome was generated by the formation of tumor-specific contigs based on the identified somatic SVs in the tumor genome. For each breakpoint-junction that includes a 5′-end of known gene, the DNA segment containing the 5′ end of the gene up to the breakpoint was joined to the flanking region on the other side of the breakpoint junction and this region was extended to a specific maximum value (often set at 500 Mb), or until a subsequent breakpoint-junction was encountered, upon which a further extension of the joined genomic segment was performed. The total contig size was typically set at 1.5 Mb, but can be adapted to any meaningful value. As a result of this procedure, for each gene containing a breakpoint, a new contig was formed, which was appended to the GRCh37 reference genome.

The short-read RNA sequencing data were aligned to the appended human reference genome using STAR (https://github.com/alexdobin/STAR), to obtain a list of all splice-junctions in the sequencing data. The erroneous long-read Oxford Nanopore RNA sequencing data were corrected using the short-read RNA-sequencing data using TALC (https://www.biorxiv.org/content/10.1101/2020.01.10.901728v2.full). Subsequently, the corrected long-read RNA sequencing data were aligned to the same appended reference using Minimap2 (FIG. 30). The alignment file (BAM) of the corrected long-read RNA sequencing data was used together with the short-read splice junctions to correct the long-read RNA splice junctions using FLAIR (https://github.com/BrooksLabUCSC/flair), since the long-read splice junctions may be off by one or a few bases because of the errors in the Nanopore sequencing. For each aligned and corrected Nanopore read bridging a breakpoint junction, the translation start site of the fused 5′-gene-end was taken to translate the aligned segments into a protein sequence. The C-terminal novel part of the protein sequence that is extending beyond the known fused 5′-gene-end is regarded as the Frame sequence (FIG. 30). A full Framome of this lung tumor is depicted in FIG. 31.

Example 10. Use of long-read RNA sequencing improves the identification of Frames in tumors. In the present disclosure, a method is described to identify Frame neoantigens in tumor cells, based on a combination of whole genome and transcriptome sequencing. The preferred methods outlined here, include the use of long-read sequencing of full-length mRNAs to identify Frame neoantigens. There are at least 3 reasons why long and/or full-length transcript reads are strongly improving the discovery of Frame neoantigens in tumor cells. First, alternative splice variants of gene transcripts are immediately resolved from full-length mRNA sequencing (FIG. 34). In addition, quantification of each alternative transcript isoform is immediately evident. Based on short-read RNA-sequencing, the exact order and connection between exons cannot be determined, as only the connections between consecutive exons are detected, while the full-length contiguity of each possible isoform remains unresolved. Since a proportion of Frame peptide sequences is encoded by multiple (novel) exons downstream of the breakpoint-junction, understanding the connectivity of the exons is of essence to determine the exact Frame structure.

Secondly, genes in the human genome may contain exons with two possible translation frames, depending on the exact isoform in which the exon resides. In addition, exons may be either classified as coding, or 3′UTR or 5′UTR, depending on their transcript isoform context. Based on gene and transcript annotation described in the Ensembl database (www.ensembl.org), for almost 20% of the exons the exon annotation is ambiguous if the isoform context is not known (FIG. 35).

Determining the transcript isoform of the 5′-gene within a chimeric transcript, is required to be able to determine the reading frame of the last exon before the breakpoint-junction that is spliced to exons downstream of the breakpoint-junction. Since long and full-length mRNA reads directly resolve the isoform structure and thus enable the identification of the reading frame of the 5′ gene involved in the chimeric transcript, the downstream novel Frame peptide sequence can be determined following the translation of novel exons downstream of the breakpoint junction, within the same frame as dictated by the 5′ portion of the known gene.

A third reason to use long-read mRNA sequencing in addition (or as alternative) to short-read RNA sequencing, concerns the mapping ambiguity or errors that may occur when mapping the partial exons contained in short RNA-sequencing reads. Short-read RNA-seq reads that are erroneously split-mapped across breakpoint-junctions can lead to false-positive Frame predictions, thereby decreasing accuracy of Frame predictions.

Example 11. Comparison of different methods for sequencing long transcripts from tumor cells. To determine the value of different long-read transcript sequencing methods for identification of Frame neoantigen sequences, isolated total RNA from a lung tumor sample and divided the isolated RNA into a portion from which we only extracted polyadenylated mRNAs, and a portion from which we isolated Capped and polyadenylated mRNAs, as described in example 8 and 9. The two mRNA preparations were converted into cDNA and sequenced on a Oxford Nanopore MinION instrument, reaching a throughput of 12.3M reads and 15.4M reads, respectively. The reads for each sequencing run were mapped to the human reference genome (GRCh37), which was appended with new contigs containing the genomic rearrangement breakpoint junctions (tumor-specific reference sequences). Following correction of the long-read Nanopore sequencing data with short-read data, Frames were identified (FIG. 32, FIG. 33). We observed that using a selection for long mRNAs using both poly-A selection and CAP selection substantially improves the identification of Frame neoantigens. For the lung tumor described in this example, two additional Frames were identified, and for other genomic structural variants, multiple Frame peptide sequences were identified, which were derived from alternative splice isoforms. A further major factor that contributes to the reliability of Frame discovery from Capped and polyadenylated mRNAs, is the fact that both a 5′ Cap and a 3′ poly-(A) signal are required for efficient translation of mRNAs into proteins (Sachs, A. The role of poly-(A) in the translation and stability of mRNA. Current Opinion in Cell Biology vol. 2 1092-1098 (1990) and Ramanathan, A., Robb, G. B. & Chan, S.-H. mRNA capping: biological functions and applications. Nucleic Acids Res. 44, 7511-7526 (2016)). Thus, selecting for Capped and polyadenylated mRNAs, increases the likelihood that a translated product is emerging as tumor-specific immune target from the identified chimeric mRNA transcripts covering somatic genomic structural variation breakpoint-junctions.

Example 12. Reconstruction of a local tumor-specific reference genome for identification of Frames in tumors. The genome of cancer cells can be heavily rearranged as a result of genomic structural variants. These genomic structural variations can give rise to novel transcripts that may encode cancer neoantigens (referred to as Frames herein). Such novel transcripts may be identified by short-read and/or long-read RNA sequencing, as outlined in examples 8 and 9. However, during analytical procedures that represent the current state-of-the-art for analysis of RNA sequencing data in cancer genomics and bioinformatics, the RNA sequencing reads are mapped to a reference genome, which is most often the latest version of the human reference genome as assembled by the Human Genome Reference Consortium. Examples of such reference genomes include GRCh37 or GRCh38, which represent two different versions of the human reference genome. However, since the cancer genome has been complexly rearranged, resulting transcripts derived from such complexly rearranged regions may also be rearranged. Hence mapping such rearranged transcript sequencing reads to the normal human reference genome (GRCh37, or the like), will complicate the mapping, because the order and orientation of the exonic sequences in the transcript reads are different than their order and orientation in said human reference genome.

To accurately identify the novel transcript structures that emerge from cancer genomic structural variations, in particular complex genomic structural variations, reconstruction of a tumor-specific reference genome represents an important first step for alignment of long and short RNA sequencing reads. An example of complex novel transcript structures emerging from rearranged segments of the human reference genome is provided in FIG. 39. In this figure a tumor sample was sequenced using whole genome sequencing and long-read and short-read RNA sequencing as outlined in examples 8 and 9. An intragenic tandem duplication was identified in the KLF5 gene. The RNA sequencing data were mapped using Minimap2 to the reconstructed tumor-specific reference genome, containing the rearranged KLF5 gene. This procedure immediately detected a long transcript sequence read alignment that identified the order and splicing of the tandemly duplicated genomic segment enabling the detection of a tumor-specific Frame neoantigen. As a comparison RNA sequencing data were also aligned to the normal (non-rearranged) KLF5 gene, which failed to detect the novel junction between tandemly duplicated exons (FIG. 40).

Example 13. Reconstruction of complexly rearranged chromothripsis regions using long-read data and comparison to short-read reconstruction. Cancer genome sequencing is typically performed using short-read next-generation sequencing, such as the sequencing provided by Illumina. The throughput and quality of Illumina sequencing makes it a strong method for reliable identification of different types of genetic changes in cancer genomes, such as point mutations and short-insertions and deletions or simple genomic structural variations. However, to fully resolve complexly rearranged regions in cancer genomes, long-range sequence information is required. Complex rearrangements, such as chromothripsis, occur at high frequency in many cancer types (Cortes-Ciriano, I. et al. Comprehensive analysis of chromothripsis in 2,658 human cancers using whole-genome sequencing. doi:10.1101/333617) and are a potentially important source of tumor neoantigens (Mansfield, A. S. et al. Neoantigenic Potential of Complex Chromosomal Rearrangements in Mesothelioma. J. Thorac. Oncol. 14, 276-287 (2019). An example of a complexly rearranged genomic region in AML (Acute Myeloid Leukemia) is depicted in FIG. 36. This region of about 4 Mb, contains 102 genomic rearrangement breakpoint junctions providing a possible source of Frame neoantigens. Using the short-read breakpoint-junctions as a starting point, we attempted to define possible rearranged contigs covering each gene that contains one or more somatic genomic breakpoints. The amount of possible contigs increases rapidly with the number of crossed breakpoint-junctions, providing an enormous amount of theoretically possible contig configurations (FIG. 37). A long-read sequencing approach is the only method by which such configuration can be resolved without making a priori assumptions on the order in which the individual junctions occur. To achieve this, we performed Oxford Nanopore sequencing of the complex genomic rearrangements in a tumor sample. We reached a read length N50 of around 15 kb, and a genomic coverage of 10×, providing multiple long Nanopore reads spanning somatic breakpoint-junctions. Nanopore reads spanning multiple breakpoint junctions were included in the process of defining tumor-specific contigs, thereby considerably reducing the number of possible contig configurations for use in subsequent mapping of long-read cDNA sequencing (FIG. 38). Further improvements to the length of the sequence reads of long-read genome sequencing allows complete reconstruction of complexly rearranged regions and subsequent identification of the entire reservoir of Frame neoantigens within such regions.

Example 14. Ribosome profiling demonstrates the translation of Frame-encoding novel transcripts. Ribosome profiling, also known as Ribo-seq, is a known sequencing method that enables the detection of translated RNA molecules and the reading frames in which the RNA molecules are translated (for review see, e.g. Calviello L. and Ohler U., Trends in Genetics 2017, https://doi.org/10.1016/j.tig.2017.08.003). In essence, Ribo-seq sequences RNA fragments that are bound by ribosomes, and hence are protected from nucleolytic degradation. We used mouse MC38 tumor tissue for a Ribo-seq, according to established procedures in the field. The P-site or peptidyl-site is the exact codon location where the peptidyl tRNA is formed in the ribosome. The offset between the 5′ site of the RNA sequence reads (derived from ribosome protected fragments) and the P-site can be calculated, e.g. with the tool Plastid. The calculated offsets are used to determine the exact genomic position of the P-site associated with each Ribo-seq RNA sequence read. The P-site coverage can be calculated across the genome, thereby identifying translation abundance of mRNAs, as well as the reading frames in which mRNAs are translated. Based on the Ribo-seq data from mouse MC38, we confirmed that genes without any genetic mutation are translated according to their expected reading frame (as described in e.g. the Ensembl database). However, for genes containing somatic frame-shift mutations, often two reading frames were observed in the mRNA following the position of the frame-shift mutation, which indicates that besides the canonical reading frame, an alternative reading frame is used for mRNAs containing the frame-shift mutation. In addition, we explored the reading frames determined by the Ribo-seq data for novel transcripts spanning genomic rearrangement breakpoint junctions. The Ribo-seq data confirmed the predicted reading frame, as derived from the known 5′-gene portions included in said novel transcripts, thereby demonstrating that the predicted Frame neopeptide sequences emerging from genomic rearrangements are existing as such.

### Example 15 Hidden Frame Neoantigens Expressed in Human Tumor Specimens

Tumor samples of various cancer types (lung, pancreas, head & neck), obtained from resections were analyzed using a combination of multiple sequencing technologies. Genomic DNA was extracted from the tumor sample and the corresponding blood cells of the same patient, using established procedures (Macherey Nagel NuceoSpin or Qiagen DNeasy spin columns). DNA was used for whole genome paired-end sequencing (2×150 bp reads) on Illumina NovaSeq instruments to an average coverage depth of 100× for the tumor sample and 30× for the corresponding blood (control) sample.

In addition, total RNA was isolated from the tumor sample using Macherey Nagel NucleoSpin RNA extraction methods. Total RNA was used for short-read RNA sequencing on Illumina NovaSeq, following ribosomal RNA depletion of total RNA and preparation of a short-read RNA sequencing library from the ribosomal RNA depleted RNA using Illumina TruSeq protocols. Approximately 50 million short paired-end RNA sequencing reads were generated per tumor sample.

Long-read full-length cDNA sequencing was performed using Oxford Nanopore GridION technology. Full-length mRNA molecules were selected from total RNA preparations obtained from tumor cells based on the presence of a 5′CAP and a 3′ poly-A tail. Double-stranded cDNA was prepared from said full length mRNA molecules and the cDNA was sequenced on Oxford Nanopore GridION or MinION or PromethION using standard procedures known to skilled persons in the art. At least 20 million full-length transcripts sequences were generated for each tumor sample.

Whole genome sequencing data were analysed using existing bioinformatics methods to identify somatic genetic changes (e.g. as described by Priestley et al, Nature 575, pages 210-216, 2019), typically resulting in a few thousand somatic point mutations (single nucleotide variations), a few hundred somatic small insertions and deletions (indels), and up to a few hundred of somatic genomic rearrangements (structural variations), per tumor sample.

Long-read cDNA (transcript) sequence reads were mapped to a tumor-specific reference genome, as determined based on the detected somatic genomic rearrangement breakpoint-junctions in the tumor genome (FIG. 26), and as described herein in FramePro. Transcript reads were identified that contain a 5′part of a known gene and an unknown portion, spanning one or more somatic rearrangement breakpoint-junctions present in the tumor genome. By this analysis, novel chimeric transcript structures were identified, which bring a non-coding sequence in the reading frame of the 5′ portion of a known gene (FIG. 8). In all cases examined, the novel portion of the transcript was spliced based on cryptic splice-donor and splice-acceptor sites. Based on the analysis set forth herein, we discovered that for approximately 10% of all somatic structural genomic rearrangements present in a tumor genome, such aberrant (chimeric) transcripts are observed.

Based on short-read RNA-sequencing of the same tumor specimens, the erroneous long-read cDNA transcript reads were polished to reach accurate and long transcript reads. Splice-junctions in the transcript reads were further polished based on the splice-junctions observed in the short RNA-sequencing reads. Translation of each individual polished transcript sequence was performed, by using the human reference genome as a default sequence for each of the identified exons in each transcript, resulting in novel chimeric proteins, consisting of a part of a known human protein and a novel amino acid sequence (referred to herein as Hidden Frame Neoantigen, FIG. 8).

A single genomic rearrangement in the tumor genome (or a series of connected genomic rearrangements) may give rise to multiple novel chimeric splice isoforms, encoding multiple novel Hidden Frame protein sequences (FIG. 30). Thus, a single tumor-specific genomic rearrangement may contribute to a large amount of neoantigenic sequences in a tumor.

Based on the procedure described above, hidden Frames were detected in multiple tumor samples, including, AML, lung, pancreas and head and neck cancers. Between 0-49 hidden Frames were detected per tumor specimen, altogether encompassing up to 1450 amino acids per tumor sample (FIG. 42). To determine the value of hidden Frames as immunotherapy targets, the number of amino acids encompassed by hidden Frames was compared to the number of mutated amino acids resulting from point mutations (missense mutations) and exonic frame-shift indels, for different tumor samples from lung and head and neck cancer. For more than 50% of the tumor samples analyzed, hidden Frames contribute the majority of neoantigenic (tumor-specific) amino acids, as compared to exonic frame-shift indels and missense mutations (FIG. 43).

Of note, chimeric transcripts emerging from tumor-specific genomic rearrangements have been described in earlier work in mesothelioma (Mansfield et al, J Thorac Oncol. 2019 February; 14(2):276-287), yet the full structure of the transcripts and their coding capacity has not been established before. Herein, it is discovered for the first time, that novel capped and polyadenylated chimeric mRNAs can be identified which are a result of genomic rearrangements in cancer genomes and are abundantly present in many different types of human tumors. These transcripts lead to neoantigenic peptides that form immediate targets of immunotherapy, such as therapeutic cancer vaccines or T-cell-based immunotherapies across a wide range of cancers.

### Example 16

There are multiple possible strategies to detect tumor-specific neoantigens (in particular Hidden Frames) and the translated products thereof, from cDNA (or RNA) sequencing data and whole genome sequencing data of the tumor. Herein, three methods for discovering neoantigens and their translated products are described.

A first approach is to directly translate each identified chimeric full-length cDNA read into a protein sequence, by starting at the annotated translation start codon from the known 5′ partner gene in the chimeric cDNA transcript read (FIG. 44). This approach is problematic because long cDNA (or RNA) sequence reads generated with sequencing platforms such as Oxford Nanopore sequencing are erroneous, with error rate between 5-10%, leading to mistakes in the translated protein sequence. Such errors in long cDNA reads may be overcome by performing circular consensus sequencing or with hybrid correction.

A second approach for determining accurate sequences of full-length chimeric transcripts encoding neoantigens, involves mapping of long RNA sequence reads (obtained from sequencing RNA or cDNA) to the human reference genome, followed by concatenating the aligned segments from the reference genome to produce a high-quality transcript sequence that can be immediately translated in a protein. Herein, we describe two approaches for neoantigen determination that involve alignment to the human reference genome.

A first method is described herein as FramePro and is extensively discussed in the previous examples. This method comprises:

a) performing whole genome sequencing of a tumor sample and a healthy sample from the individual, optionally performing long-read whole genome sequencing of a tumor sample and a healthy sample from the individual,

b) performing long-read RNA sequencing on RNA or long-read sequencing on the corresponding cDNA from at least one tumor sample, preferably wherein RNA is poly-(A) selected mRNA and/or 5′ cap containing mRNA;

c) optionally performing short-read RNA sequencing on RNA or short-read sequencing on the corresponding cDNA from at least one tumor sample;

d) mapping the genomic sequences obtained from the tumor tissue and corresponding healthy tissue to a human reference sequence to identify structural genomic variations in the tumor sample,

e) generating in silico a reconstructed tumor-specific reference genome comprising the identified somatic structural genomic variations;

f) aligning the RNA sequences to the reconstructed tumor genome;

g) determining the sequences of the full-length RNA transcripts encoded by nucleic acid sequences comprising the somatic structural genomic variations;

h) determining the predicted amino acid sequences encoded by the full-length transcripts of g),

i) selecting, as candidate neoantigen sequences, sequences comprising at least 9 contiguous amino acids of the predicted amino acid sequence of h), wherein at least four of the contiguous amino acids are not encoded in the germline genome of the individual.

A second method, termed ‘direct-RNA Frame detection’ utilizes the mapping of long, hybrid-correct/polished RNA sequence reads (obtained from sequencing RNA or cDNA) to a normal human reference genome, such as GRCh37, GRCh38 or the like, followed by identification of a possible ‘path’ following genomic rearrangement breakpoint-junctions in the tumor genome that could lead to a contig that places the mapped cDNA/RNA segments together in a small genomic sequence (arbitrarily defined as smaller than e.g. 200 kb) (FIG. 7). Such method is particularly relevant for identification of hidden Frames emerging from complex genomic. Briefly, the method comprises:

a) performing whole genome sequencing of a tumor sample and a healthy sample from the individual, optionally performing long-read whole genome sequencing of a tumor sample and a healthy sample from the individual,

b) performing long-read RNA sequencing on RNA or long-read sequencing on the corresponding cDNA from at least one tumor sample, preferably wherein RNA is poly-(A) selected mRNA and/or 5′ cap containing mRNA;

c) optionally performing short-read RNA sequencing on RNA or short-read sequencing on the corresponding cDNA from at least one tumor sample;

d) aligning the RNA sequence reads (obtained from sequencing RNA or cDNA) to a human reference sequence;

e) mapping the genomic sequences obtained from the tumor tissue and corresponding healthy tissue to a human reference sequence to identify structural genomic variations in the tumor sample,

f) identification of a linear contig of DNA sequence from the tumor genomic sequences that comprises a structural genomic variation and comprises genomic segments that align to the RNA sequence reads (obtained from sequencing RNA or cDNA); e) generating in silico a reconstructed tumor-specific reference genome comprising the identified somatic structural genomic variations to which the RNA/cDNA sequences align;

f) aligning the RNA sequence reads to the reconstructed tumor-specific reference genome;

g) determining the sequences of the full-length RNA transcripts encoded by nucleic acid sequences comprising the somatic structural genomic variations;

h) determining the predicted amino acid sequences encoded by the full-length transcripts of g),

i) selecting, as candidate neoantigen sequences, sequences comprising at least 9 contiguous amino acids of the predicted amino acid sequence of h), wherein at least four of the contiguous amino acids are not encoded in the germline genome of the individual.

### Example 17

To illustrate the use of multiple methods for detection of hidden Frames, a human solid tumor sample was sequenced using a combination of whole-genome sequencing, short-read RNA sequencing and long-read RNA sequencing. Sequencing data processing was performed according to ‘reconstructed tumor genome mapping’ and ‘direct RNA Frame detection’ described herein and hidden Frames were detected (FIG. 45). The Hidden Frames detected according to both methods were overlapping for the vast majority (36 Frames derived from 15 structural variation loci). In addition, 10 Frames were uniquely detected based on the ‘direct RNA Frame detection’ method. These data illustrate that a multi-analytical approach improves the sensitivity of detection of hidden Frames in tumor genomes.

1. A method for identifying neoantigen sequences,
wherein the method identifies mRNA transcripts resulting from DNA rearrangements that form new junctions of DNA sequences, wherein the DNA rearrangements result in the fusion of at least part of the coding strand of a first gene to intergenic non-coding DNA or to the noncoding strand of a second gene, said method comprising:
performing whole genome sequencing of a tumor sample and a healthy sample from an individual,
optionally performing long-read whole genome sequencing of a tumor sample and a healthy sample from the individual,
performing long-read RNA sequencing on RNA or long-read sequencing on the corresponding cDNA from at least one tumor sample;
optionally performing short-read RNA sequencing on RNA or short-read sequencing on the corresponding cDNA from at least one tumor sample;
identifying somatic DNA rearrangements in the tumor sample;
determining the sequence of the full-length RNA transcripts encoded by nucleic acid sequences comprising or overlapping with the DNA rearrangements;
determining the amino acid sequences encoded by the full-length transcripts,
selecting, as candidate neoantigen sequences, sequences comprising at least 9 contiguous amino acids of the amino acid sequence encoded by the full-length transcripts, wherein at least four of the contiguous amino acids are not encoded in the germline genome of the individual.

2. The method of claim 1, wherein the method also identifies:
mRNA transcripts derived from intragenic frameshift mutations in polypeptide encoding sequences, wherein the frameshift mutations results, in a change of the reading frame of said polypeptide encoding sequence, and
mRNA transcripts resulting from DNA rearrangements that form new junctions of DNA sequences, wherein the DNA rearrangement results in the fusion of at least part of the coding strand of a first gene to at least part of the coding strand of a second gene or the rearrangement is an intragenic genomic rearrangement, wherein said DNA rearrangement results in a change of the reading frame of a polypeptide encoding sequence.

3. The method of claim 1 for identifying candidate neoantigen sequences, said method comprising:
a) performing whole genome sequencing of a tumor sample and a healthy sample from the individual,
optionally performing long-read whole genome sequencing of a tumor sample and a healthy sample from the individual,
b) performing long-read RNA sequencing on RNA or long-read sequencing on the corresponding cDNA from at least one tumor sample to obtain RNA sequencing reads;
c) optionally performing short-read RNA sequencing on RNA or short-read sequencing on the corresponding cDNA from at least one tumor sample;
d) mapping the genomic sequences obtained from the tumor tissue and corresponding healthy tissue to a human reference sequence to identify DNA rearrangements in the tumor sample,
e) generating in silico a reconstructed tumor-specific reference genome comprising the identified somatic DNA rearrangements;
f) aligning the RNA sequencing reads to the reconstructed tumor-specific reference genome;
g) determining the sequences of the full-length RNA transcripts encoded by nucleic acid sequences comprising the somatic DNA rearrangements;
h) determining the amino acid sequences encoded by the full-length transcripts of g),
i) selecting, as candidate neoantigen sequences, sequences comprising at least 9 contiguous amino acids of the amino acid sequence of h), wherein at least four of the contiguous amino acids are not encoded in the germline genome of the individual.

4. The method of claim 1 for identifying candidate neoantigen sequences, said method comprising:
a) performing whole genome sequencing of a tumor sample and a healthy sample from the individual,
optionally performing long-read whole genome sequencing of a tumor sample and a healthy sample from the individual,
b) performing long-read RNA sequencing on RNA or long-read sequencing on the corresponding cDNA from at least one tumor sample to obtain RNA sequencing reads;
c) optionally performing short-read RNA sequencing on RNA or short-read sequencing on the corresponding cDNA from at least one tumor sample;
d) aligning the RNA sequencing reads to a human reference sequence;
e) mapping the genomic sequences obtained from the tumor tissue and corresponding healthy tissue to a human reference sequence to identify DNA rearrangements in the tumor sample,
f) identification of a linear contig of DNA sequence from the tumor genomic sequences that comprises a DNA rearrangement and comprises genomic segments that align to RNA sequencing reads;
g) generating in silico a reconstructed tumor-specific reference genome comprising the identified DNA rearrangement to which the RNA sequencing reads align;
h) aligning the RNA sequencing reads to the reconstructed tumor-specific reference genome;
i) determining the sequences of the full-length RNA transcripts encoded by nucleic acid sequences comprising the somatic DNA rearrangements;
j) determining the amino acid sequences encoded by the full-length transcripts of i),
k) selecting, as candidate neoantigen sequences, sequences comprising at least 9 contiguous amino acids of the amino acid sequence of j), wherein at least four of the contiguous amino acids are not encoded in the germline genome of the individual.

5. The method of claim 1, wherein the RNA sequencing is performed using long-read direct RNA sequencing.

6. The method of claim 1, wherein the method further comprises selecting poly-(A) mRNA from said tumor sample and performing long-read RNA sequencing or long-read cDNA sequencing based on the poly-(A) selected mRNA.

7. A method for preparing a vaccine or collection of vaccines for the treatment of cancer in an individual, comprising identifying candidate neoantigen peptide sequences according to claim 1 and preparing a vaccine or collection of vaccines comprising peptides having said amino acid sequences or comprising nucleic acids encoding said amino acid sequences.

8. The method of claim 7 wherein the candidate neoantigen peptide sequences comprise amino acid sequences encoded by DNA rearrangements resulting in new junctions of DNA sequences, wherein the rearrangement results in the fusion at least part of the coding strand of a first gene to intergenic non-coding DNA or to the noncoding strand of a second gene.

9. The method of claim 1, wherein the candidate neoantigen peptide sequences comprise amino acid sequences encoded by intragenic frameshift mutations in polypeptide encoding sequences, wherein the mutation results in a change of the reading frame of said polypeptide encoding sequence and/or DNA rearrangements resulting in new junctions of DNA sequences, wherein the rearrangement results in the fusion of at least part of the coding strand of a first gene to at least part of the coding strand of a second gene or the rearrangement is an intragenic genomic rearrangement, wherein said DNA rearrangement results in a change of the reading frame of a polypeptide encoding sequence.

10. The method of claim 7, wherein said method comprises
i) selecting from the candidate neoantigen peptide sequences identified, neoantigen peptide sequences having one or more of the following characteristics:
neoantigen peptide sequences which do not share a contiguous stretch of at least 6 amino acids with human protein reference sequences;
neoantigen peptide sequences wherein the genomic variant allele frequency of the respective somatic mutation in the tumor cells of a tumor sample is at least 0.1;
neoantigen peptide sequences wherein the cysteine content for each peptide is 30% or less, where cysteine content (Qcys) is defined as the number of cysteines in said sequence divided by the total number of amino acids in said sequence;
neoantigen peptide sequences for which the underlying somatic mutations have a maximum distance with regard to chromosomal location; and
neoantigen peptide sequences wherein the peptides are predicted to comprise one or more MHC I and/or MHC II binding epitopes; and
ii) preparing a vaccine or collection of vaccines comprising peptides having the selected neoantigen amino acid sequences or nucleic acids encoding the selected amino acid sequences.

11. The method of claim 7, wherein said vaccine or collection of vaccines comprises essentially all candidate neoantigen peptides identified, or nucleic acids encoding said peptides.

12. The method of claim 7, wherein the vaccine or collection of vaccines comprises at least 100 amino acids corresponding to the candidate neoantigen peptide sequences encoded by the new open reading frames.

13. The method of claim 1, wherein the cancer is not MSI.

14. A vaccine or collection of vaccines for the treatment of cancer, obtainable by a method according to claim 7, wherein the vaccine comprises a neoantigen peptide, or nucleic acid encoding said neoantigen peptide, wherein the neoantigen peptide comprise amino acid sequences encoded by DNA rearrangements resulting in new junctions of DNA sequences, wherein the rearrangement results in the fusion at least part of the coding strand of a first gene to intergenic non-coding DNA or to the noncoding strand of a second gene.

15. A vaccine or collection of vaccines for the treatment of cancer, wherein the vaccine comprises at least two different neoantigen peptides, or nucleic acid encoding said neoantigen peptides, wherein each neoantigen peptide comprise amino acid sequences encoded by DNA rearrangements resulting in new junctions of DNA sequences, wherein the rearrangement results in the fusion at least part of the coding strand of a first gene to intergenic non-coding DNA or to the noncoding strand of a second gene.

16. The vaccine or collection of vaccines of claim 15, wherein at least two different neoantigen peptides are linked.

17.-19. (canceled)

20. A method for the treatment of cancer comprising administering to an individual in need thereof a vaccine or collection of vaccines, wherein the vaccine comprises a neoantigen peptide, or nucleic acid encoding said neoantigen peptide, wherein the neoantigen peptide comprise amino acid sequences encoded by DNA rearrangements resulting in new junctions of DNA sequences, wherein the rearrangement results in the fusion at least part of the coding strand of a first gene to intergenic non-coding DNA or to the noncoding strand of a second gene.

21. A method for preparing a cellular immunotherapy for the treatment of cancer in an individual, said method comprising contacting T-cells with MHC-I molecules bound to one or more of the candidate neoantigen peptide sequences identified from the individual according to claim 1.

22. The method according to claim 21, wherein the T-cells are obtained from said individual.

23. The method according to claim 21, wherein said contacting results in the stimulation of the T-cells.

24.-26. (canceled)


SEQUENCE LISTING (7 sequences)

SEQ ID NO: 1 - DNA, 51 bp, Artificial Sequence
  normal frame
acatgaggatctgatcgcatggggcactttagagctactgtaagtaattct

SEQ ID NO: 2 - DNA, 49 bp, Artificial Sequence
  frame shift
acatgaggatctgatcgcatggcactttagagctactgtaagtaattct

SEQ ID NO: 3 - DNA, 47 bp, Artificial Sequence
  normal frame
acatgaggatctgatcgcatggggcaacattaggtgttccctacgaa

SEQ ID NO: 4 - DNA, 23 bp, Artificial Sequence
  out-of-frame
caacattaggtgttccctacgaa

SEQ ID NO: 5 - DNA, 24 bp, Artificial Sequence
  normal frame
acatgaggatctgatcgcatgggg

SEQ ID NO: 6 - DNA, 23 bp, Artificial Sequence
  out-of-frame
caacattaggtgttccctacgaa

SEQ ID NO: 7 - protein, 109 aa, Artificial Sequence
  Class III frame
VSPPFFRLPRGRPGLRAVGRRRSWRRAGVAARLWQVCLGRGGQRGRGGGRRGRGRKDSFGSGEGRLSAACQLFLLCSSCFPHLDLIFFPRQVFQGDDPFSLSLPLGGED